SEQUENCE LISTING (4 sequences)

SEQ ID NO: 1 - protein, 419 aa, Artificial sequence
  Synthetic
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - protein, 6 aa, Artificial sequence
  Synthetic
HHHHHH

SEQ ID NO: 3 - protein, 5 aa, Artificial sequence
  Synthetic
DDDDK

SEQ ID NO: 4 - protein, 6 aa, Artificial sequence
  Synthetic
ADDDDK

(19) United States
(12) Patent Application Publication
(10) Pub. No.: US 2022/0043000 A1
(43) Pub. Date: Feb. 10, 2022

(54) METHODS AND KITS FOR DETECTING SARS-COV-2 PROTEIN IN A SAMPLE

(71) Applicant: Abbott Laboratories, Abbott Park, IL (US)

(72) Inventors: A. Scott Muerhoff, Abbott Park, IL (US); Toru Yoshimura, Tokyo (JP); Philip M. Hemken, Abbott Park, IL (US); Eitan Israeli, Abbott Park, IL (US); Ryotaro Chiba, Matsudo (JP); Takamitsu Morikawa, Matsudo (JP); Dong Wang, Matsudo (JP); Fusamitsu Yanagihara, Matsudo (JP)

(21) Appl. No.: 17/393,824

(22) Filed: Aug. 4, 2021

Related U.S. Application Data
(60) Provisional application No. 63/156,775, filed on Mar. 4, 2021.
(60) Provisional application No. 63/060,922, filed on Aug. 4, 2020.

(51) Int. Cl. G01N 33/58; G01N 33/569

(52) U.S. Cl. CPC G01N 33/582; G01N 2800/26; G01N 33/56983

(57) ABSTRACT

Disclosed herein are methods, kits, and systems relates for detecting the presence or determining the amount of SARS-CoV-2 nucleocapsid protein in one or more samples obtained from a subject.

### RELATED APPLICATION INFORMATION

This application claims priority to U.S. Application No. 63/060,922 filed Aug. 4, 2020 and U.S. Application No. 63/156,775, filed on Mar. 4, 2021, the contents of which are herein incorporated by reference.

### SEQUENCE LISTING

Incorporated by reference in its entirety herein is a computer-readable nucleotide/amino acid sequence listing submitted concurrently herewith and identified as follows: One 4,460 Byte ASCII (Text) file named “38688-203_ST25.TXT,” created on Aug. 4, 2021.

### TECHNICAL FIELD

The present disclosure relates methods, kits, and systems for detecting the presence or determining the amount of at least one SARS-CoV-2 protein (e.g, antigen) in one or more samples obtained from a subject. In some aspects, the methods, kits and systems employ at least one polycation which improves the sensivity of detecting the presence or determining the amount of at least one SARS-CoV-2 nucleocapsid protein in one or more samples obtained from a subject.

### BACKGROUND

Viruses of the family Coronaviridae possess a single-strand, positive-sense RNA genome ranging from 26 to 32 kilobases in length (reviewed by Lu et al., The Lancet, 395:565-574 (Feb. 22, 2020)). The Coronaviridae are further subdivided (initially based on serology but now based on phylogenetic clustering) into four groups, the alpha, beta, gamma and delta coronaviruses. Coronaviruses have been identified in several avian hosts, as well as in various mammals, including camels, bats, masked palm civets, mice, dogs, and cats.

Among the several coronaviruses that are pathogenic to humans, most are associated with mild clinical symptoms, with three exceptions. Severe acute respiratory syndrome (SARS) coronavirus (SARS-CoV) is a novel betacoronavirus that emerged in Guangdong, southern China, in November 2002 and resulted in more than 8000 human infections and 774 deaths in 37 countries in 2002-03. Middle East respiratory syndrome (MERS) coronavirus (MERS-CoV) was first detected in Saudi Arabia in 2012 and was responsible for 2494 laboratory-confirmed cases of infection and 858 deaths from 2012-20. In December, 2019, a cluster of pneumonia cases caused by a newly identified β-coronavirus were found to be epidemiologically-associated with the Huanan seafood market in Wuhan, China, where a number of non-aquatic animals, such as birds and rabbits were on sale before the outbreak. This coronavirus was named January 2020 by the World Health Organization (WHO) as the 2019-novel coronavirus (2019-nCov or COVID-19), and February 2020 by the International Committee as SARS-CoV-2. SARS-CoV-2 was declared a pandemic due to its rapid, uncontrolled and vast worldwide spread.

Coronavirus virions are spherical with diameters of approximately 125 nanometers, as demonstrated in studies by cryo-electron tomography and cryo-electron microscopy. A prominent feature of coronaviruses is the club-shape spike projections emanating from the surface of the virion, giving the virion the appearance of a solar corona and resulting in the name, coronaviruses. Within the envelope of the coronavirus virion is the helically-symmetrical nucleocapsid, which binds to and creates a shell around the coronavirus RNA genome. The spike (S) and nucleocapsid (N) proteins are the main immunogens of the coronavirus. The other two main structural proteins of the coronavirus particles are the membrane (M) and envelope (E) proteins. All four proteins are encoded within the 3′ end of the viral genome.

The S protein (˜150 kDa) is heavily N-linked glycosylated and utilizes an N-terminal signal sequence to gain access to the endoplasmic reticulum (ER). Homotrimers of the virus-encoding S protein make up the distinctive spike structure on the surface of the virus. In many, but not all, coronaviruses, the S protein is cleaved by a host cell furin-like protease into two separate polypeptides noted S1 and S2. S1 makes up the large receptor-binding domain of the S protein while S2 forms the stalk of the spike molecule. The trimeric S glycoprotein mediates attachment of the coronavirus virion to the host cell by interactions between the S protein and its receptor. In humans, angiotensin-converting enzyme 2 (ACE2) is the receptor for SARS-CoV. The sites of receptor binding domains (RBD) within the S1 region of a coronavirus S protein vary depending on the virus, with some having the RBD at the N-terminus of S1 (e.g., murine hepatitis virus) while others (e.g., SARS-CoV) have the RBD at the C-terminus of S1. The S-protein/receptor interaction is the primary determinant for the coronavirus to infect a host species and also governs the tissue tropism of the virus.

The M protein is the most abundant structural protein in the virion. It is a small (˜25-30 kDa) protein with 3 transmembrane domains and is believed to give the virion its shape. It has a small N-terminal glycosylated ectodomain and a much larger C-terminal endodomain that extends 6-8 nm into the viral particle. The E protein (˜8-12 kDa) is found in small quantities within the virion. E proteins in coronaviruses are highly divergent but have a common architecture. Data suggests that the E protein is a transmembrane protein with an N-terminal ectodomain and a C-terminal endodomain that has ion channel activity. Recombinant viruses lacking the E protein are not always lethal—although this is virus-type dependent. The E protein facilitates assembly and release of the virus, but also has other functions (e.g., ion channel activity in SARS-CoV E protein is not required for viral replication but is required for pathogenesis).

The N protein is the only protein present in the nucleocapsid. It is composed of two separate domains, an N-terminal domain (NTD) and a C-terminal domain (CTD), both capable of binding RNA in vitro using different mechanisms, which may suggest that optimal RNA binding requires contributions from both domains. The N protein is heavily phosphorylated, and phosphorylation has been suggested to trigger a structural change enhancing the affinity for viral versus non-viral RNA. The N protein binds the viral genome in a beads-on-a-string type conformation. Two specific RNA substrates have been identified for N protein; the transcriptional regulatory sequences and the genomic packaging signal. The genomic packaging signal has been found to bind specifically to the second, or C-terminal RNA binding domain. The N protein also binds nsp3, a key component of the replicase complex, and the M protein. These protein interactions likely help tether the viral genome to the replicase-transcriptase complex, and subsequently package the encapsidated genome into viral particles.

In February 2020, Lu et al. reported obtaining complete and partial SARS-CoV-2 genome sequences using next-generation sequencing of bronchoalveolar lavage fluid samples and cultured isolates from nine patients from Wuhan diagnosed with viral pneumonia but negative for common respiratory pathogens. Lu et al., The Lancet, 395: 565-574 (Feb. 22, 2020). Based on their analysis, Lu et al. further reported that SARS-CoV-2 was closely related (with 88% identity) to two bat-derived severe acute respiratory syndrome (SARS)-like coronaviruses, bat-SL-CoVZC45 and bat-SL-CoVZXC21, collected in eastern China in 2018, but was more distant from SARS-CoV (about 79%) and MERS-CoV (about 50%). Additionally, Zhou et al. confirmed that SARS-CoV-2 uses the same cellular entry receptor, ACE2, as SARS-CoV. Zhou et al., Nature, 579:270-273 (March 2020).

SARS-CoV-2 primarily spreads through the respiratory tract, by droplets, respiratory secretions, and direct contact. Additionally, SARS-CoV-2 has been found in fecal swabs and blood, indicating the possibility of multiple routes of transmission. Zhang et al., Microbes 9(1):386-9 (2020). SARS-CoV-2 is highly transmissible in humans, especially in the elderly and people with underlying diseases. Symptoms can appear 2 to 14 days after exposure. Patients present with symptoms such as fever, malaise, cough, and/or shortness of breath. Most adults or children with SARS-CoV-2 infection present with mild flu-like symptoms, however, critical patients rapidly develop acute respiratory distress syndrome, respiratory failure, multiple organ failure and even death.

Because of the health risks imposed by SARS-CoV-2 transmission, there is a need for methods and kits to assess coronavirus transmission in humans, including methods to determine the presence and/or detect the amount of SARS-CoV-2 protein in one or more samples obtained from a subject.

### SUMMARY

In one aspect, the present disclosure relates to an improvement of a method of detecting a presence or determining an amount of a SARS-CoV-2 nucleocapsid protein in a biological sample, wherein the method comprises detecting at least one complex comprising a first specific binding partner, said sample SARS-CoV-2 nucleocapsid protein, and a second specific binding partner comprising at least one detectable label and further wherein the first specific binding partner, second specific binding partner, or the first specific binding partner and the second specific binding partner comprise at least one anti-SARS-CoV antibody, anti-SARS-CoV-2 antibody, or fragment thereof that specifically binds to said sample SARS-CoV-2 nucleocapsid protein, wherein the improvement comprises allowing the complex to form in the presence of at least one polycation having a molecular weight of at least about 500 daltons or greater prior to assessing the signal from the complex, wherein the amount of detectable signal from the detectable label in the complex indicates the presence or amount of SARS-CoV-2 nucleocapsid protein in the sample.

In some aspects, the biological sample in the above method is whole blood, serum, plasma, saliva, an oropharyngeal specimen, or a nasopharyngeal specimen. Specifically, in some aspects, the biological sample is whole blood. In other aspects, the biological sample is plasma. In yet other aspects, the biological sample is saliva. In still other aspects, the biological sample is an oropharyngeal specimen. In still yet other aspects, the biological sample is a nasopharyngeal specimen.

In still yet other aspects, the first specific binding partner comprises at least one anti-SARS-CoV antibody, anti-SARS-CoV-2 antibody, or fragment thereof, or the first specific binding partner and the second specific binding partner each comprise at least one anti-SARS-CoV antibody, anti-SARS-CoV-2 antibody or fragment thereof.

In still further aspects, the polycation in the above method is at least one polylysine, at least one polyornithine, at least one poly-L-histidine, at least one poly-L-arginine, at least one polyethylenimine, at least one DEAE-Dextran, or combinations thereof. More specifically, in some aspects, the (i) the polylysine is poly-L-lysine hydrobromide, poly-D-lysine hydrobromide, poly-L-lysine hydrochloride, poly-L-lysine trifluoroacetate, poly(lysine, alanine) 3:1 hydrobromide, poly(lysine, arginine) 2:1 hydrobromide, poly(lysine, alanine) 1:1 hydrobromide, or poly(lysine, tryptophan) 1:4 hydrobromide; (ii) the polyornithine is poly-L-ornithine hydrobromide or poly-DL-ornithine hydrobromide; (iii) the poly-L-histidine is poly-L-histidine hydrobromide; and (iv) the poly-L-arginine is poly-L-arginine hydrochloride or poly-L-arginine hydrobromide.

In still further aspects, the improvement increases sensitivity by at least about 5%, at least about 10%, at least about 15%, at least about 20%, at least about 25%, at least about 30%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 95%, at least about 100%, at least about 110%, at least about 120%, at least about 125%, at least about 130%, at least about 140%, at least about 150%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% when compared to methods which do not use or employ at least one polycation.

In still further aspects, the method can be selected from the group consisting of an immunoassay, a clinical chemistry assay, a point-of-care assay, and a lateral flow assay. In yet other aspects, the method can be performed using single molecule detection. In still other aspects, the method is adapted for use in an automated system or a semi-automated system.

In yet another aspect, the present disclosure relates to a method of detecting a presence or determining an amount of a SARS-CoV-2 nucleocapsid protein in a biological sample in a subject. The method can comprise:
a) contacting at least one biological sample from the subject, either simultaneously or sequentially, in any order, with:
   at least one first specific binding partner comprising at least one anti-SARS-CoV antibody, anti-SARS-CoV-2 antibody, or fragment thereof that specifically binds to at least one SARS-CoV-2 nucleocapsid protein in the sample,
   at least one second specific binding partner comprising at least one detectable label, thereby producing one or more complexes comprising the first binding member-SARS-CoV-2 nucleocapsid protein-second specific binding partner, and
   at least one polycation having a molecular weight of at least about 500 daltons or greater; and
b) assessing a signal from the one or more complexes, wherein the amount of detectable signal from the detectable label indicates the presence or amount of SARS-CoV-2 nucleocapsid protein in the sample.

In some aspects, the biological sample in the above method is whole blood, serum, plasma, saliva, an oropharyngeal specimen, or a nasopharyngeal specimen. Specifically, in some aspects, the biological sample is whole blood. In other aspects, the biological sample is plasma. In yet other aspects, the biological sample is saliva. In still other aspects, the biological sample is an oropharyngeal specimen. In still yet other aspects, the biological sample is a nasopharyngeal specimen.

In still yet other aspects, the first specific binding partner comprises at least one anti-SARS-CoV antibody, anti-SARS-CoV-2 antibody, or fragment thereof, or the first specific binding partner and the second specific binding partner each comprise at least one anti-SARS-CoV antibody, anti-SARS-CoV-2 antibody or fragment thereof.

In still further aspects, the polycation used in the above method is at least one polylysine, at least one polyornithine, at least one poly-L-histidine, at least one poly-L-arginine, at least one polyethylenimine, at least one DEAE-Dextran, or combinations thereof. More specifically, in some aspects, the (i) the polylysine is poly-L-lysine hydrobromide, poly-D-lysine hydrobromide, poly-L-lysine hydrochloride, poly-L-lysine trifluoroacetate, poly(lysine, alanine) 3:1 hydrobromide, poly(lysine, arginine) 2:1 hydrobromide, poly(lysine, alanine) 1:1 hydrobromide, or poly(lysine, tryptophan) 1:4 hydrobromide; (ii) the polyornithine is poly-L-ornithine hydrobromide or poly-DL-ornithine hydrobromide; (iii) the poly-L-histidine is poly-L-histidine hydrobromide; and (iv) the poly-L-arginine is poly-L-arginine hydrochloride or poly-L-arginine hydrobromide.

In still further aspects, the improvement increases sensitivity by at least about 5%, at least about 10%, at least about 15%, at least about 20%, at least about 25%, at least about 30%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 95%, at least about 100%, at least about 110%, at least about 120%, at least about 125%, at least about 130%, at least about 140%, at least about 150%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% when compared to methods which do not use or employ at least one polycation.

In still yet other aspects, the method can be selected from the group consisting of an immunoassay, a clinical chemistry assay, a point-of-care assay, and a lateral flow assay. In yet other aspects, the method can be performed using single molecule detection. In still other aspects, the method is adapted for use in an automated system or a semi-automated system.

In yet another aspect, the present disclosure relates to a kit for performing the above method. Specifically, the kit comprises:
a. at least one specific binding partner comprising at least one anti-SARS-CoV antibody, anti-SARS-CoV-2 antibody, or fragment thereof that specifically binds to at least one SARS-CoV-2 nucleocapsid protein;
b. at least one second specific binding partner comprising at least one detectable label; and
c. at least one polycation having a molecular weight of at least about 500 daltons or greater.

In other aspects, the kit further comprises, or is configured to be used with at least one calibrator reagent, at least one control reagent, or at least one calibrator reagent and at least one control reagent. In some aspects, the kit further comprises at least one solid support. In yet other aspects, the polycation in the kit is at least one polylysine, at least one polyornithine, at least one poly-L-histidine, at least one poly-L-arginine, at least one polyethylenimines, at least one DEAE-Dextran, or combinations thereof. Specifically, in some aspects, the (i) the polylysine is poly-L-lysine hydrobromide, poly-D-lysine hydrobromide, poly-L-lysine hydrochloride, poly-L-lysine trifluoroacetate, poly(lysine, alanine) 3:1 hydrobromide, poly(lysine, arginine) 2:1 hydrobromide, poly(lysine, alanine) 1:1 hydrobromide, or poly(lysine, tryptophan) 1:4 hydrobromide; (ii) the polyornithine is poly-L-ornithine hydrobromide or poly-DL-ornithine hydrobromide; (iii) the poly-L-histidine is poly-L-histidine hydrobromide; and (iv) the poly-L-arginine is poly-L-arginine hydrochloride or poly-L-arginine hydrobromide. In some aspects, the kit is adapted for use with an automated or semi-automated system.

In yet another aspect, the disclosure relates to a system for detecting a presence or determining an amount of a SARS-CoV-2 nucleocapsid protein in a biological sample in a subject. The system comprises:
at least one first specific binding partner comprising at least anti-SARS-CoV antibody, anti-SARS-CoV-2 antibody, or fragment thereof that specifically binds to at least one SARS-CoV-2 nucleocapsid protein in the sample;
at least one second specific binding partner comprising at least one detectable label, thereby producing one or more complexes comprising the first binding member-SARS-CoV-2 nucleocapsid protein-second specific binding partner;
at least one polycation having a molecular weight of at least about 500 daltons or greater; and
at least one device for detecting the at least one label from the complex.

In some aspects, in the system, the device for detecting the label from the complex is automated or semi-automated. In other aspects, in the system, the polycation is at least one polylysine, at least one polyornithine, at least one poly-L-histidine, at least one poly-L-arginine, at least one polyethylenimines, at least one DEAF-Dextran, or combinations thereof. In some aspects, the (i) the polylysine is poly-L-lysine hydrobromide, poly-D-lysine hydrobromide, poly-L-lysine hydrochloride, poly-L-lysine trifluoroacetate, poly(lysine, alanine) 3:1 hydrobromide, poly(lysine, arginine) 2:1 hydrobromide, poly(lysine, alanine) 1:1 hydrobromide, or poly(lysine, tryptophan) 1:4 hydrobromide; (ii) the polyornithine is poly-L-ornithine hydrobromide or poly-DL-ornithine hydrobromide; (iii) the poly-L-histidine is poly-L-histidine hydrobromide; and (iv) the poly-L-arginine is poly-L-arginine hydrochloride or poly-L-arginine hydrobromide.

### BRIEF DESCRIPTION OF THE DRAWINGS

FIG. 1 provides a sequence listing comprising SEQ ID NOS. 1-4.

### DETAILED DESCRIPTION

The present disclosure relates to methods, kits, and systems to detect the presence of or determine the amount, concentration and/or level of at least one SARS-CoV-2 protein (e.g., antigen), such as at least one SARS-CoV-2 nucleocapsid protein, in a sample. In some aspects, the methods, kits, and systems described herein are used to detect the presence of or determine the amount, concentration and/or level of at least one SARS-CoV-2 nucleocapsid protein in samples with improved sensitivity (e.g., have a higher signal to noise (S/N) ratio) than other methods, kits, and systems. In one aspect, the disclosure relates to an improvement in a method of detecting a presence or determining an amount of a SARS-CoV-2 nucleocapsid protein in a biological sample, wherein the method involves adding at least one polycation having a molecular weight of at least about 500 daltons or greater to the sample prior to detecting the presence or determing the amount of at least one SARS-CoV-2 nucleocapsid protein in the sample. The addition of at least one polycation having a molecular weight of at least about 500 daltons or greater to the sample results in an improvement in the sensitivity (e.g., S/N ratio) of detection in the method of at least about 5%, at least about 10%, at least about 15%, at least about 20%, at least about 25%, at least about 30%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 95%, at least about 100%, at least about 110%, at least about 120%, at least about 125%, at least about 130%, at least about 140%, at least about 150%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% when compared to methods which do not use or employ at least one polycation.

In another aspect, the disclosure relates to methods of detecting the presence or determining an amount of at least one SARS-CoV-2 nucleocapsid protein in a biological sample by contacting at least one sample obtained from a subject (either simultaneously or sequentially, in any order), with at least one first specific binding partner comprising at at least one anti-SARS-CoV antibody, anti-SARS-CoV-2 antibody, or fragment thereof that specifically binds to at least one SARS-CoV-2 nucleocapsid protein in the sample, at least one second specific binding partner comprising a detectable label to produce one or more complexes comprising the first specific binding partner-SARS-CoV-2 nucleocapsid protein-second specific binding partner, and at least one polycation having a molecule weight of at least about 500 daltons or greater. A signal from the one or more complexes are assessed (e.g., detected). Specifically, the amount of the detectable signal from the detectable label indicates the presence or amount of SARS-CoV-2 nucleocapsid protein in the sample. The addition of at least one polycation having a molecular weight of at least about 500 daltons or greater in the method improves the sensitivity (e.g., S/N ratio) of the method by at least about 5%, at least about 10%, at least about 15%, at least about 20%, at least about 25%, at least about 30%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 95%, at least about 100%, at least about 110%, at least about 120%, at least about 125%, at least about 130%, at least about 140%, at least about 150%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% when compared to methods which do not use or employ at least one polycation.

The biological sample used in the methods of the present disclosure may be obtained from an asymptomatic subject or from a subject exhibiting one or more symptoms of infection with SARS-CoV-2. The methods of the present disclosure also include treating a subject identified as having a SARS-CoV-2 and optionally, monitoring such subjects, such as before, during and/or after receiving such treatments.

In another aspect, the present disclosure relates to kits for performing such methods.

In still yet another aspect, the present disclosure relates to systems for detecting at least one SARS-CoV-2 nucleocapsid protein in a biological sample.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“At least” herein refers to type and/or quantity, unless it is evident from the context in which the clause is applied that it refers to only type, or only quantity, and not both type and quantity.

“Affinity matured antibody” is used herein to refer to an antibody with one or more alterations in one or more CDRs, which result in an improvement in the affinity (i.e., K_{D}, k_{d} or kₐ) of the antibody for a target antigen compared to a parent antibody, which does not possess the alteration(s). Exemplary affinity matured antibodies will have nanomolar or even picomolar affinities for the target antigen. A variety of procedures for producing affinity matured antibodies is known in the art, including the screening of a combinatory antibody library that has been prepared using bio-display. For example, Marks et al., BioTechnology, 10: 779-783 (1992) describes affinity maturation by VH and VL domain shuffling. Random mutagenesis of CDR and/or framework residues is described by Barbas et al., Proc. Nat. Acad. Sci. USA, 91: 3809-3813 (1994); Schier et al., Gene, 169: 147-155 (1995); Yelton etal., J. Immunol., 155: 1994-2004 (1995); Jackson et al., J. Immunol., 154(7): 3310-3319 (1995); and Hawkins et al, J. Mol. Biol., 226: 889-896 (1992). Selective mutation at selective mutagenesis positions and at contact or hypermutation positions with an activity-enhancing amino acid residue is described in U.S. Pat. No. 6,914,128 B1.

“Antibody” and “antibodies” as used herein refers to monoclonal antibodies, monospecific antibodies (e.g., which can either be monoclonal, or may also be produced by other means than producing them from a common germ cell), multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain variable fragments (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), or domain antibodies (dAbs) (e.g., such as described in Holt et al. (2014) Trends in Biotechnology 21:484-490), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody”.

“Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Bead” and “particle” are used herein interchangeably and refer to a substantially spherical solid support. One example of a bead or particle is a microparticle. Microparticles that can be used herein can be any type known in the art. For example, the bead or particle can be a magnetic bead or magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO2, MnAs, MnBi, Eu0, and NiO/Fe. Examples of ferrimagnetic materials include NiFe₂O₄, CoFe₂O₄, Fe₃O₄ (or FeO.Fe₂O₃). Beads can have a solid core portion that is magnetic and is surrounded by one or more non-magnetic layers. Alternately, the magnetic portion can be a layer around a non-magnetic core. The microparticles can be of any size that would work in the methods described herein, e.g., from about 0.75 to about 5 nm, or from about 1 to about 5 nm, or from about 1 to about 3 nm.

“Binding protein” is used herein to refer to a monomeric or multimeric protein that binds to and forms a complex with a binding partner, such as, for example, a polypeptide, an antigen, a chemical compound or other molecule, or a substrate of any kind. A binding protein specifically binds a binding partner. Binding proteins include antibodies, as well as antigen-binding fragments thereof and other various forms and derivatives thereof as are known in the art and described herein below, and other molecules comprising one or more antigen-binding domains that bind to an antigen molecule or a particular site (epitope) on the antigen molecule. Accordingly, a binding protein includes, but is not limited to, an antibody a tetrameric immunoglobulin, an IgG molecule, an IgG1 molecule, a monoclonal antibody, a chimeric antibody, a CDR-grafted antibody, a humanized antibody, an affinity matured antibody, and fragments of any such antibodies that retain the ability to bind to an antigen.

“Bispecific antibody” is used herein to refer to a full-length antibody that is generated by quadroma technology (see Milstein et al., Nature, 305(5934): 537-540 (1983)), by chemical conjugation of two different monoclonal antibodies (see, Staerz et al., Nature, 314(6012): 628-631 (1985)), or by knob-into-hole or similar approaches, which introduce mutations in the Fc region (see Holliger et al., Proc. Natl. Acad. Sci. USA, 90(14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences), and is monovalent for each antigen to which it binds to.

As used herein, the term “coronavirus” refers to viruses that belonging to the family Coronaviridae that have a positive-sense, RNA genome ranging from 26 to 32 kilobases in length. Coronaviruses having four main structural proteins: the spike glycoprotein (S protein), the membrane protein (M protein), the envelope protein (E protein) and the nucleocapsid protein (N protein). Coronavirus can be further subdivided into four groups, alpha, beta, gamma and delta coronaviruses. Examples of alpha coronaviruses include HCoV-229E and HCoV-NL63. Examples of beta coronaviruses are HCoV-OC43, HCoV-HKU1, Middle East Respiratory Syndrome (MFRS-CoV), severe acute respiratory syndrome (SARS) coronavirus (SARS-CoV) and SARS-CoV-2 (also known as 2019-nCov, COVID-19, coronavirus disease, and Coronavirus Disease 2019).

In one aspect, the present disclosure relates to β-coronaviruses. In another aspect, the β-coronaviruses are MERS-CoV, SARS-CoV and SARS-CoV-2. In still yet another aspect, the β-coronaviruses are SARS-CoV and SARS-CoV-2. In still yet another aspect, the β-coronavirus is SARS-CoV-2. The sequence of SARS-CoV-2 has been described in a number of publications, such as, for example, Lu et al., Lancet, 395:565-574 (February 2020) and https://www.ncbi.nlm.nih.gov/genbank/sars-cov-2-seqs/, the contents of each are herein incorporated by reference.

“CDR” is used herein to refer to the “complementarity determining region” within an antibody variable sequence. There are three CDRs in each of the variable regions of the heavy chain and the light chain. Proceeding from the N-terminus of a heavy or light chain, these regions are denoted “CDR1”, “CDR2”, and “CDR3”, for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196: 901-917 (1987); and Chothia et al., Nature, 342: 877-883 (1989)) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan, FASEB J., 9: 133-139 (1995), and MacCallum, J. Mol. Biol., 262(5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat- or Chothia-defined CDRs.

“Component,” “components,” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, whole blood, serum or plasma sample, in accordance with the methods described herein and other methods known in the art. Some components can be in solution or lyophilized for reconstitution for use in an assay

“Controls” as used herein generally refers to a reagent whose purpose is to evaluate the performance of a measurement system in order to assure that it continues to produce results within permissible boundaries (e.g., boundaries ranging from measures appropriate for a research use assay on one end to analytic boundaries established by quality specifications for a commercial assay on the other end). To accomplish this, a control should be indicative of patient results and optionally should somehow assess the impact of error on the measurement (e.g., error due to reagent stability, calibrator variability, instrument variability, and the like). As used herein, a “control subject” relates to a subject or subjects that has not been infected with a coronavirus, such as, a β-coronavirus (i.e., SARS-CoV-2) or been exposed to any subject that has had a coronavirus, such as a β-coronavirus (i.e., SARS-CoV-2).

As used herein, the term “control zone” or “control line” is a region of a test strip in which a label can be observed to shift location, appear, change color, or disappear to indicate that an assay performed correctly. Detection or observation of the control zone (e.g., of a control line) may be done by any convenient means, depending upon the particular choice of label, especially, for example but not limited to, visually, fluorescently, by reflectance, radiographically, and the like. As will be described, the label may or may not be applied directly to the control zone, depending upon the design of the control being used.

“Determined by an assay” is used herein to refer to the determination of a reference level by any appropriate assay. The determination of a reference level may, in some embodiments, be achieved by an assay of the same type as the assay that is to be applied to the sample from the subject (for example, by an immunoassay, clinical chemistry assay, a single molecule detection assay, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS)). The determination of a reference level may, in some embodiments, be achieved by an assay of the same type and under the same assay conditions as the assay that is to be applied to the sample from the subject. As noted herein, this disclosure provides exemplary reference levels (e.g., calculated by comparing reference levels at different time points). It is well within the ordinary skill of one in the art to adapt the disclosure herein for other assays to obtain assay-specific reference levels for those other assays based on the description provided by this disclosure. For example, a set of training samples comprising samples obtained from subjects known to have been infected by a coronavirus, such as a β-coronavirus, and samples obtained from human subjects known not to have been infected with a coronavirus, such as a β-coronavirus, or been exposed to a subject that has been infected with a coronavirus, such as a β-coronavirus (i.e., SARS-CoV-2), may be used to obtain assay-specific reference levels. It will be understood that a reference level “determined by an assay” and having a recited level of “sensitivity” and/or “specificity” is used herein to refer to a reference level which has been determined to provide a method of the recited sensitivity and/or specificity when said reference level is adopted in the methods of the disclosure. It is well within the ordinary skill of one in the art to determine the sensitivity and specificity associated with a given reference level in the methods of the disclosure, for example by repeated statistical analysis of assay data using a plurality of different possible reference levels.

Practically, when discriminating between a subject as having been infected by a coronavirus, such as a β-coronavirus (i.e., SARS-CoV-2), or not having been infected by a coronavirus, such as a β-coronavirus, the skilled person will balance the effect of raising a cutoff on sensitivity and specificity. Raising or lowering a cutoff will have a well-defined and predictable impact on sensitivity and specificity, and other standard statistical measures. It is well known that raising a cutoff will improve specificity but is likely to worsen sensitivity (proportion of those with disease who test positive). In contrast, lowering a cutoff will improve sensitivity but will worsen specificity (proportion of those without disease who test negative). The ramifications for detecting or measuring a coronavirus, such as a β-coronavirus (i.e., SARS-CoV-2), will be readily apparent to those skilled in the art. In discriminating whether a subject has or has not been infected by a coronavirus, such as a β-coronavirus, the higher the cutoff, specificity improves as more true negatives (i.e., subjects not having been infected by a coronavirus, such as β-coronavirus (e.g., SARS-CoV-2)) are distinguished from those having been infected by a coronavirus, such as a β-coronavirus. But at the same time, raising the cutoff decreases the number of cases identified as positive overall, as well as the number of true positives, so the sensitivity must decrease. Conversely, the lower the cutoff, sensitivity improves as more true positives (i.e., subjects having been infected with a coronavirus, such as a β-coronavirus) are distinguished from those who have not been infected (e.g., do not have) with a coronavirus, such as a β-coronavirus (i.e., SARS-CoV-2). But at the same time, lowering the cutoff increases the number of cases identified as positive overall, as well as the number of false positives, so the specificity must decrease.

Generally, a high sensitivity value helps one of skill rule out disease or condition (such as infection with a coronavirus, such as a β-coronavirus (i.e., SARS-CoV-2)), and a high specificity value helps one of skill rule in disease or condition. Whether one of skill desires to rule out or rule in disease depends on what the consequences are for the patient for each type of error. Accordingly, one cannot know or predict the precise balancing employed to derive a test cutoff without full disclosure of the underlying information on how the value was selected. The balancing of sensitivity against specificity and other factors will differ on a case-by-case basis. This is why it is sometimes preferable to provide alternate cutoff (e.g., reference) values so a physican or practitioner can choose.

“Dual-specific antibody” is used herein to refer to a full-length antibody that can bind two different antigens (or epitopes) in each of its two binding arms (a pair of HC/LC) (see PCT publication WO 02/02773). Accordingly, a dual-specific binding protein has two identical antigen binding arms, with identical specificity and identical CDR sequences, and is bivalent for each antigen to which it binds.

“Dual variable domain” is used herein to refer to two or more antigen binding sites on a binding protein, which may be divalent (two antigen binding sites), tetravalent (four antigen binding sites), or multivalent binding proteins. DVDs may be monospecific, i.e., capable of binding one antigen (or one specific epitope), or multispecific, i.e., capable of binding two or more antigens (i.e., two or more epitopes of the same target antigen molecule or two or more epitopes of different target antigens). A preferred DVD binding protein comprises two heavy chain DVD polypeptides and two light chain DVD polypeptides and is referred to as a “DVD immunoglobulin” or “DVD-Ig.” Such a DVD-Ig binding protein is thus tetrameric and reminiscent of an IgG molecule, but provides more antigen binding sites than an IgG molecule. Thus, each half of a tetrameric DVD-Ig molecule is reminiscent of one half of an IgG molecule and comprises a heavy chain DVD polypeptide and a light chain DVD polypeptide, but unlike a pair of heavy and light chains of an IgG molecule that provides a single antigen binding domain, a pair of heavy and light chains of a DVD-Ig provide two or more antigen binding sites.

Each antigen binding site of a DVD-Ig binding protein may be derived from a donor (“parental”) monoclonal antibody and thus comprises a heavy chain variable domain (VH) and a light chain variable domain (VL) with a total of six CDRs involved in antigen binding per antigen binding site. Accordingly, a DVD-Ig binding protein that binds two different epitopes (i.e., two different epitopes of two different antigen molecules or two different epitopes of the same antigen molecule) comprises an antigen binding site derived from a first parental monoclonal antibody and an antigen binding site of a second parental monoclonal antibody.

A description of the design, expression, and characterization of DVD-Ig binding molecules is provided in PCT Publication No. WO 2007/024715, U.S. Pat. No. 7,612,181, and Wu et al., Nature Biotech., 25: 1290-1297 (2007). A preferred example of such DVD-Ig molecules comprises a heavy chain that comprises the structural formula VD1-(X1)n-VD2-C-(X2)n, wherein VD1 is a first heavy chain variable domain, VD2 is a second heavy chain variable domain, C is a heavy chain constant domain, X1 is a linker with the proviso that it is not CH1, X2 is an Fc region, and n is 0 or 1, but preferably 1; and a light chain that comprises the structural formula VD1-(X1)n-VD2-C-(X2)n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1, but preferably 1. Such a DVD-Ig may comprise two such heavy chains and two such light chains, wherein each chain comprises variable domains linked in tandem without an intervening constant region between variable regions, wherein a heavy chain and a light chain associate to form tandem functional antigen binding sites, and a pair of heavy and light chains may associate with another pair of heavy and light chains to form a tetrameric binding protein with four functional antigen binding sites. In another example, a DVD-Ig molecule may comprise heavy and light chains that each comprise three variable domains (VD1, VD2, VD3) linked in tandem without an intervening constant region between variable domains, wherein a pair of heavy and light chains may associate to form three antigen binding sites, and wherein a pair of heavy and light chains may associate with another pair of heavy and light chains to form a tetrameric binding protein with six antigen binding sites.

In a preferred embodiment, a DVD-Ig binding protein not only binds the same target molecules bound by its parental monoclonal antibodies, but also possesses one or more desirable properties of one or more of its parental monoclonal antibodies. Preferably, such an additional property is an antibody parameter of one or more of the parental monoclonal antibodies. Antibody parameters that may be contributed to a DVD-Ig binding protein from one or more of its parental monoclonal antibodies include, but are not limited to, antigen specificity, antigen affinity, potency, biological function, epitope recognition, protein stability, protein solubility, production efficiency, immunogenicity, pharmacokinetics, bioavailability, tissue cross reactivity, and orthologous antigen binding.

A DVD-Ig binding protein binds at least one epitope of nucleocapsid protein, spike protein or nucleocapsid protein and spike protein from a coronavirus, such as a β-coronavirus. Non-limiting examples of a DVD-Ig binding protein include a DVD-Ig binding protein that binds one or more epitopes of a nucleocapsid protein, spike protein, or nucleocapsid protein and spike protein of a β-coronavirus, such as SARS-CoV-2, a DVD-Ig binding protein that binds an epitope of a human nucleocapsid protein, spike protein, or nucleocapsid protein and spike protein of a β-coronavirus, such as SARS-CoV-2, and an epitope of a nucleocapsid protein, spike protein, or nucleocapside protein and spike protein of a β-coronavirus (i.e., such as SARS-CoV-2) of another species (for example, mouse, rat, bat, etc.), and a DVD-Ig binding protein that binds an epitope of a human β-coronavirus and an epitope of another target molecule.

“Dynamic range” as used herein refers to range over which an assay readout is proportional to the amount of target molecule or analyte in the sample being analyzed.

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

“Fragment antigen-binding fragment” or “Fab fragment” as used herein refers to a fragment of an antibody that binds to antigens and that contains one antigen-binding site, one complete light chain, and part of one heavy chain. Fab is a monovalent fragment consisting of the VL, VH, CL and CH1 domains. Fab is composed of one constant and one variable domain of each of the heavy and the light chain. The variable domain contains the paratope (the antigen-binding site), comprising a set of complementarity determining regions, at the amino terminal end of the monomer. Each arm of the Y thus binds an epitope on the antigen. Fab fragments can be generated such as has been described in the art, e.g., using the enzyme papain, which can be used to cleave an immunoglobulin monomer into two Fab fragments and an Fc fragment, or can be produced by recombinant means.

“F(ab′)₂ fragment” as used herein refers to antibodies generated by pepsin digestion of whole IgG antibodies to remove most of the Fc region while leaving intact some of the hinge region. F(ab′)₂ fragments have two antigen-binding F(ab) portions linked together by disulfide bonds, and therefore are divalent with a molecular weight of about 110 kDa. Divalent antibody fragments (F(ab′)₂ fragments) are smaller than whole IgG molecules and enable a better penetration into tissue thus facilitating better antigen recognition in immunohistochemistry. The use of F(ab′)₂ fragments also avoids unspecific binding to Fc receptor on live cells or to Protein A/G. F(ab′)₂ fragments can both bind and precipitate antigens.

“Framework” (FR) or “Framework sequence” as used herein may mean the remaining sequences of a variable region minus the CDRs. Because the exact definition of a CDR sequence can be determined by different systems (for example, see above), the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3, and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region.

Human heavy chain and light chain FR sequences are known in the art that can be used as heavy chain and light chain “acceptor” framework sequences (or simply, “acceptor” sequences) to humanize a non-human antibody using techniques known in the art. In one embodiment, human heavy chain and light chain acceptor sequences are selected from the framework sequences listed in publicly available databases such as V-base (hypertext transfer protocol://vbase.mrc-cpe.cam.ac.uk/) or in the international ImMunoGeneTics® (IMGT®) information system (hypertext transfer protocol://imgt.cines.fr/texts/IMGTrepertoire/LocusGenes/).

“Functional antigen binding site” as used herein may mean a site on a binding protein (e.g., an antibody) that is capable of binding a target antigen. The antigen binding affinity of the antigen binding site may not be as strong as the parent binding protein, e.g., parent antibody, from which the antigen binding site is derived, but the ability to bind antigen must be measurable using any one of a variety of methods known for evaluating protein, e.g., antibody, binding to an antigen. Moreover, the antigen binding affinity of each of the antigen binding sites of a multivalent protein, e.g., multivalent antibody, herein need not be quantitatively the same.

The term “fusion protein” as used herein relates to a protein or polypeptide comprising at least one first protein or polyeptide joined or linked to at least one second protein or polypeptide. In some aspects, the at least one protein or polypeptide is joined or linked to at least one second protein or polypeptide through one or more linking peptide sequences. An example of a fusion proteion is a chimeric protein. A fusion protein can be created using routine techniques known in the art such as recombinant DNA technology, through joining or linking of two or more genes that originally coded for separate proteins. Thus, a fusion protein may comprise a multimer of different or identical binding proteins which are expressed as a single, linear polypeptide.

“Humanized antibody” is used herein to describe an antibody that comprises heavy and light chain variable region sequences from a non-human species (e.g., a mouse) but in which at least a portion of the VH and/or VL sequence has been altered to be more “human-like,” i.e., more similar to human germline variable sequences. A “humanized antibody” is an antibody or a variant, derivative, analog, or fragment thereof, which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98%, or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains the light chain as well as at least the variable domain of a heavy chain. The antibody also may include the CHL hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

A humanized antibody can be selected from any class of immunoglobulins, including IgM, IgG, IgD, IgA, and IgE, and any isotype, including without limitation IgG1, IgG2, IgG3, and IgG4. A humanized antibody may comprise sequences from more than one class or isotype, and particular constant domains may be selected to optimize desired effector functions using techniques well-known in the art.

The framework regions and CDRs of a humanized antibody need not correspond precisely to the parental sequences, e.g., the donor antibody CDR or the consensus framework may be mutagenized by substitution, insertion, and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see, e.g., Winnaker, From Genes to Clones (Verlagsgesellschaft, Weinheim, 1987)). A “consensus immunoglobulin sequence” may thus comprise a “consensus framework region(s)” and/or a “consensus CDR(s)”. In a family of immunoglobulins, each position in the consensus sequence is occupied by the amino acid occurring most frequently at that position in the family. If two amino acids occur equally frequently, either can be included in the consensus sequence.

“Identical” or “identity,” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence. As used herein, “isolated polypeptide” refers to a polypeptide (e.g., of recombinant, synthetic or chemical original or a combination thereof), that, by virtue of its origin, the isolated polypeptide is not associated with all or a portion of a polypeptide and/or other protein(s) with which the “isolated polypeptide” is found in nature; is operably linked to a polypeptide and/or protein that it is not linked to in nature; or does not occur in nature as part of a larger sequence. When associated with a particular species, virus or strain (e.g., “β-coronavirus isolated polypeptide” or “SARS-CoV-2 polypeptide”), the isolated polypeptide optionally can be made by recombinant means rather than by isolation from in vivo.

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

Any suitable detectable label as is known in the art can be used. For example, the detectable label can be a radioactive label (such as 3H, 14C, 32P, 33P, 35S, 90Y, 99Tc, 111In, 125I, 131I, 177Lu, 166Ho, and 153 Sm), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oregon. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16: 1324-1328 (2006); Adamczyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)).

In one aspect, the acridinium compound is an acridinium-9-carboxamide. Methods for preparing acridinium 9-carboxamides are described in Mattingly, J. Biolumin. Chemilumin. 6: 107-114 (1991); Adamczyk et al., J. Org. Chem. 63: 5636-5639 (1998); Adamczyk et al., Tetrahedron 55: 10899-10914 (1999); Adamczyk et al., Org. Lett. 1: 779-781 (1999); Adamczyk et al., Bioconjugate Chem. 11: 714-724 (2000); Mattingly et al., In Luminescence Biotechnology: Instruments and Applications; Dyke, K. V. Ed.; CRC Press: Boca Raton, pp. 77-105 (2002); Adamczyk et al., Org. Lett. 5: 3779-3782 (2003); and U.S. Pat. Nos. 5,468,646, 5,543,524 and 5,783,699 (each of which is incorporated herein by reference in its entirety for its teachings regarding same).

Another example of an acridinium compound is an acridinium-9-carboxylate aryl ester. An example of an acridinium-9-carboxylate aryl ester of formula II is 10-methyl-9-(phenoxycarbonyl)acridinium fluorosulfonate (available from Cayman Chemical, Ann Arbor, Mich.). Methods for preparing acridinium 9-carboxylate aryl esters are described in McCapra et al., Photochem. Photobiol. 4: 1111-21 (1965); Razavi et al., Luminescence 15: 245-249 (2000); Razavi et al., Luminescence 15: 239-244 (2000); and U.S. Pat. No. 5,241,070 (each of which is incorporated herein by reference in its entirety for its teachings regarding same). Such acridinium-9-carboxylate aryl esters are efficient chemiluminescent indicators for hydrogen peroxide produced in the oxidation of an analyte by at least one oxidase in terms of the intensity of the signal and/or the rapidity of the signal. The course of the chemiluminescent emission for the acridinium-9-carboxylate aryl ester is completed rapidly, i.e., in under 1 second, while the acridinium-9-carboxamide chemiluminescent emission extends over 2 seconds. Acridinium-9-carboxylate aryl ester, however, loses its chemiluminescent properties in the presence of protein. Therefore, its use requires the absence of protein during signal generation and detection. Methods for separating or removing proteins in the sample are well-known to those skilled in the art and include, but are not limited to, ultrafiltration, extraction, precipitation, dialysis, chromatography, and/or digestion (see, e.g., Wells, High Throughput Bioanalytical Sample Preparation. Methods and Automation Strategies, Elsevier (2003)). The amount of protein removed or separated from the test sample can be about 40%, about 45%, about 50%, about 55%, about 60%, about 65%, about 70%, about 75%, about 80%, about 85%, about 90%, or about 95%. Further details regarding acridinium-9-carboxylate aryl ester and its use are set forth in U.S. patent application Ser. No. 11/697,835, filed Apr. 9, 2007. Acridinium-9-carboxylate aryl esters can be dissolved in any suitable solvent, such as degassed anhydrous N,N-dimethylformamide (DMF) or aqueous sodium cholate.

“Linking sequence” or “linking peptide sequence” refers to a natural or artificial polypeptide sequence that is connected to one or more polypeptide sequences of interest (e.g., full-length, fragments, etc.). The term “connected” refers to the joining of the linking sequence to the polypeptide sequence of interest. Such polypeptide sequences are preferably joined by one or more peptide bonds. Linking sequences can have a length of from about 4 to about 50 amino acids. Preferably, the length of the linking sequence is from about 6 to about 30 amino acids. Natural linking sequences can be modified by amino acid substitutions, additions, or deletions to create artificial linking sequences. Linking sequences can be used for many purposes, including in recombinant Fabs. Exemplary linking sequences include, but are not limited to: (i) Histidine (His) tags, such as a 6X His tag, which has an amino acid sequence of HHHHHH (SEQ ID NO: 2), are useful as linking sequences to facilitate the isolation and purification of polypeptides and antibodies of interest; (ii) Enterokinase cleavage sites, like His tags, are used in the isolation and purification of proteins and antibodies of interest. Often, enterokinase cleavage sites are used together with His tags in the isolation and purification of proteins and antibodies of interest. Various enterokinase cleavage sites are known in the art. Examples of enterokinase cleavage sites include, but are not limited to, the amino acid sequence of DDDDK (SEQ ID NO: 3) and derivatives thereof (e.g., ADDDDK (SEQ ID NO: 4), etc.); (iii) Miscellaneous sequences can be used to link or connect the light and/or heavy chain variable regions of single chain variable region fragments. Examples of other linking sequences can be found in Bird et al., Science 242: 423-426 (1988); Huston et al., PNAS USA 85: 5879-5883 (1988); and McCafferty et al., Nature 348: 552-554 (1990). Linking sequences also can be modified for additional functions, such as attachment of drugs or attachment to solid supports. In the context of the present disclosure, the monoclonal antibody, for example, can contain a linking sequence, such as a His tag, an enterokinase cleavage site, or both.

“Monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological.

“Multivalent binding protein” is used herein to refer to a binding protein comprising two or more antigen binding sites (also referred to herein as “antigen binding domains”). A multivalent binding protein is preferably engineered to have three or more antigen binding sites, and is generally not a naturally occurring antibody. The term “multispecific binding protein” refers to a binding protein that can bind two or more related or unrelated targets, including a binding protein capable of binding two or more different epitopes of the same target molecule.

“Nucleocapsid protein” or “N” protein as used interchangeably herein, refers to one of four main structural proteins of a coronavirus. The N protein is the only protein present in the nucleocapsid. It is composed of two separate domains, an N-terminal domain (NTD) and a C-terminal domain (CTD), both capable of binding RNA in vitro using different mechanisms, which may suggest that optimal RNA binding requires contributions from both domains. For example, in SARS-CoV-2, the NTD can be found at amino acids 1 to 209 of SEQ ID NO.1. For example, in SARS-CoV-2, the CTD can be found at amino acids 210 to 419 of SEQ ID NO.1.

In some aspects described herein, a nucleocapsid protein is at least a portion (e.g., at least 5 amino acids, at least 10 amino acids, at least 15 amino acids, at least 20 amino acids, at least 25 amino acids, at least 30 amino acids, at least 40 amino acids, at least 50 amino acids, at least 60 amino acids, at least 70 amino acids, at least 80 amino acids, at least 90 amino acids, at least 100 amino acids, at least 105 amino acfids, at least 110 amino acids, at least 115 amino acids, at least 120 amino acids, at least 125 amino acids, at least 130 amino acids, at least 135 amino acids, at least 140 amino acids, at least 145 amino acids, at least 150 amino acids, at least 160 amino acids, at least 165 amino acids, at least 170 amino acids, at least 175 amino acids, at least 180 amino acids, at least 185 amino acids, at least 190 amino acids, at least 195 amino acids, at least 200 amino acids, at least 205 amino acids, at least 210 amino acids, at least 215 amino acids, at least 220 amino acids, at least 225 amino acids, at least 230 amino acids, at least 235 amino acids, at least 240 amino acids, at least 245 amino acids, at least 250 amino acids, at least 265 amino acids, at least 270 amino acids, at least 275 amino acids, at least 280 amino acids, at least 285 amino acids, at least 290 amino acids, at least 295 amino acids, at least 300 amino acids, at least 305 amino acids, at least 310 amino acids, at least 315 amino acids, at least 320 amino acids, at least 325 amino acids, at least 330 amino acids, at least 335 amino acids, at least 340 amino acids, at least 345 amino acids, at least 350 amino acids, at least 355 amino acids, at least 360 amino acids, at least 365 amino acids, at least 370 amino acids, at least 375 amino acids, at least 380 amino acids, at least 385 amino acids, at least 390 amino acids, at least 395 amino acids, at least 400 amino acids, at least 405 amino acids, at least 410 amino acids, at least 415 amino acids, or greater) or the entirety of a nucleocapsid protein from a SARS-CoV-2 strain of a β-coronavirus comprising the sequence of SEQ ID NO:1.

“Point-of-care device” refers to a device used to provide medical diagnostic testing at or near the point-of-care (namely, outside of a laboratory), at the time and place of patient care (such as in a hospital, physician's office, urgent or other medical care facility, a patient's home, a nursing home and/or a long-term care and/or hospice facility). Examples of point-of-care devices include those produced by Abbott Laboratories (Abbott Park, Ill.) (e.g., i-STAT and i-STAT® Alinity, Universal Biosensors (Rowville, Australia) (see US 2006/0134713), Axis-Shield PoC AS (Oslo, Norway) and Clinical Lab Products (Los Angeles, USA).

A “polycation” as used herein refers to an organic, inorganic, synthetic, or naturally occurring compound that has at least two positive charges. In some aspects, the polycation has a molecular weight of about 500 daltons or greater. In some aspects, the polycation has a molecular weight of about 800 daltons or greater. In some aspects, the polycation has a molecular weight of about 1,000 daltons or greater. In some aspects, the polycation has a molecular weight of about 2,000 daltons or greater. In some aspects, the polycation has a molecular weight of about 3,000 daltons or greater, about 4,000 daltons or greater, about 5,000 daltons or greater, about 6,000 daltons or greater, about 7,000 daltons or greater, about 8,000 daltons or greater, about 9,000 daltons or greater, about 10,000 daltons or greater, about 15,000 daltons or greater, about 20,000 daltons or greater, about 25,000 daltons or greater, about 30,000 daltons or greater, about 35,000 daltons or greater, about 40,000 daltons or greater, about 45,000 daltons or greater, about 50,000 daltons or greater, about 55,000 daltons or greater, about 60,000 daltons or greater, about 65,000 daltons or greater, about 70,000 daltons or greater, about 75,000 daltons or greater, about 80,000 daltons or greater, about 85,000 daltons or greater, about 90,000 daltons or greater, about 100,000 daltons or greater, about 150,000 daltons or greater, about 200,000 daltons or greater, about 250,000 daltons or greater, about 300,000 or greater, about 350,000 daltons or greater, about 400,000 daltons or greater, about 450,000 daltons or greater, or about 500,000 daltons or greater.

In some aspects, polycations that can be used in the present disclosure include polylysines (such as poly-L-lysines and poly-D-lysines) having a molecular weight range of about 1000 daltons to about 400,000 daltons, polyornithines (such as poly-L-ornithines or poly-DL-ornithines) having a molecular weight range of about 5000 daltons to about 500,000 daltons, poly-L-histidines having a molecular weight range of about 5000 daltons to about 100,000 daltons, poly-L-arginines having a molecular weight range of about 5000 daltons to about 100,000 daltons, polyethylenimines having a molecular weight range of about 800 daltons to about 5,000 daltons, DEAE-Dextrans having a molecular weight range of about 500 daltons to about 500,000 daltons, or any combinations thereof. Examples of polylysines that can be used include poly-L-lysine hydrobromide, poly-D-lysine hydrobromide, poly-L-lysine hydrochloride, poly-L-lysine trifluoroacetate, poly(lysine,alanine) 3:1 hydrobromide, poly(lysine, arginine) 2:1 hydrobromide, poly(lysine, alanine) 1:1 hydrobromide, or poly(lysine, tryptophan) 1:4 hydrobromide. Examples of polyornithines that can be used include poly-L-ornithine hydrobromide or poly-DL-ornithine hydrobromide. Examples of poly-L-histidines that can be used include poly-L-histidine hydrobromide. Examples of poly-L-arginines that can be used include poly-L-arginine hydrochloride and poly-L-arginine hydrobromide.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a reference level or control level (e.g., “low”, “medium”, or “high” levels), can be used. Multiple calibrators (i.e., more than one calibrator or a varying amount of calibrator(s)) can be used in conjunction to comprise a “sensitivity panel.”

“Recombinant antibody” and “recombinant antibodies” refer to antibodies prepared by one or more steps, including cloning nucleic acid sequences encoding all or a part of one or more monoclonal antibodies into an appropriate expression vector by recombinant techniques and subsequently expressing the antibody in an appropriate host cell. The terms include, but are not limited to, recombinantly produced monoclonal antibodies, chimeric antibodies, humanized antibodies (fully or partially humanized), multi-specific or multi-valent structures formed from antibody fragments, bifunctional antibodies, heteroconjugate Abs, DVD-Ig®s, and other antibodies as described in (i) herein. (Dual-variable domain immunoglobulins and methods for making them are described in Wu, C., et al., Nature Biotechnology, 25:1290-1297 (2007)). The term “bifunctional antibody,” as used herein, refers to an antibody that comprises a first arm having a specificity for one antigenic site and a second arm having a specificity for a different antigenic site, i.e., the bifunctional antibodies have a dual specificity.

“Reference level” as used herein refers to an assay cutoff value (or level) that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). As used herein, the term “cutoff” refers to a limit (e.g., such as a number) above which there is a certain or specific clinical outcome and below which there is a different certain or specific clinical outcome.

This disclosure provides exemplary reference levels. However, it is well-known that reference levels may vary depending on the nature of the immunoassay (e.g., capture and detection reagents employed, reaction conditions, sample purity, etc.) and that assays can be compared and standardized. It further is well within the ordinary skill of one in the art to adapt the disclosure herein for other immunoassays to obtain immunoassay-specific reference levels for those other immunoassays based on the description provided by this disclosure. Whereas the precise value of the reference level may vary between assays, the findings as described herein should be generally applicable and capable of being extrapolated to other assays.

“Sample,” “test sample,” “specimen,” “sample from a subject,” “biological sample,” and “patient sample” as used interchangeably herein may be a sample of blood, such as whole blood (including for example, capillary blood, venous blood, dried blood spot, etc.), tissue, urine, serum, plasma, amniotic fluid, lower respiratory specimens such as, but not limited to, sputum, endotracheal aspirate or bronchoalveolar lavage, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. Additionally, the sample can be a nasopharyngeal or oropharyngeal sample obtained using one or more swabs that, once obtained, is placed in a sterile tube containing a virus transport media (VTM) or universal transport media (UTM), for testing.

A variety of cell types, tissue, or bodily fluid may be utilized to obtain a sample. Such cell types, tissues, and fluid may include sections of tissues such as biopsy and autopsy samples, oropharyngeal specimens, nasopharyngeal specimens, frozen sections taken for histologic purposes, blood (such as whole blood, dried blood spots, etc.), plasma, serum, red blood cells, platelets, interstitial fluid, cerebralspinal fluid, etc. Cell types and tissues may also include lymph fluid, cerebrospinal fluid, or any fluid collected by aspiration. A tissue or cell type may be provided by removing a sample of cells from a human and a non-human animal, but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary. In some embodiments, the sample is a whole blood sample. In some embodiments, the sample is a capillary blood sample. In some embodiments, the sample is a dried blood spot. In some embodiments, the sample is a serum sample. In yet other embodiments, the sample is a plasma sample. In some embodiments, the sample is an oropharyngeal specimen. In other embodiments, the sample is a nasopharyngeal specimen. In other embodiments, the sample is sputum. In other embodiments, the sample is endotracheal aspirate. In still yet other embodiments, the sample is bronchoalveolar lavage.

“Sensitivity” of an assay as used herein refers to the proportion of subjects for whom the outcome is positive that are correctly identified as positive (e.g., correctly identifing those subjects with a disease or medical condition for which they are being tested). For example, this might include correctly identifying subjects as having been infected with a coronavirus, such as a β-coronavirus, from those who do not have not been infected with a coronavirus, such as a β-coronavirus. In some aspects, the sensitivity of an assay can be determined by evaluating changes in the signal to noise (S/N) ratio of the assay. For example, in some aspects, an increase in a S/N ratio may indicate an improvement in the sensitivity of an assay for a particular analyte (e.g., SARS-CoV-2 nucleocapsid protein).

“Specificity” of an assay as used herein refers to the proportion of subjects for whom the outcome is negative that are correctly identified as negative (e.g., correctly identifying those subjects who do not have a disease or medical condition for which they are being tested). For example, this might include correctly identifying subjects having being infected with a coronavirus, such as a β-coronavirus, from those who have not been infected with a coronavirus, such as a β-coronavirus.

“Series of calibrating compositions” refers to a plurality of compositions comprising a known concentration of the analytes, such as one or more polypeptides (such as one or more peptides derived from SARS-CoV-2 nucleocapsid protein) wherein each of the compositions differs from the other compositions in the series by the concentration of the analytes, such as one or more SARS-CoV-2 proteins (e.g., one or more SARS-CoV-2 nucleocapsid proteins)

As used herein the term “single molecule detection” refers to the detection and/or measurement of a single molecule of an analyte in a test sample at very low levels of concentration (such as pg/mL or femtogram/mL levels). A number of different single molecule analyzers or devices are known in the art and include nanopore and nanowell devices. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety.

“Solid phase” or “solid support” as used interchangeably herein, refers to any material that can be used to attach and/or attract and immobilize (1) one or more capture agents or capture specific binding partners, or (2) one or more detection agents or detection specific binding partners. The solid phase can be chosen for its intrinsic ability to attract and immobilize a capture agent. Alternatively, the solid phase can have affixed thereto a linking agent that has the ability to attract and immobilize the (1) capture agent or capture specific binding partner, or (2) detection agent or detection specific binding partner. For example, the linking agent can include a charged substance that is oppositely charged with respect to the capture agent (e.g., capture specific binding partner) or detection agent (e.g., detection specific binding partner) itself or to a charged substance conjugated to the (1) capture agent or capture specific binding partner or (2) detection agent or detection specific binding partner. In general, the linking agent can be any binding partner (preferably specific) that is immobilized on (attached to) the solid phase and that has the ability to immobilize the (1) capture agent or capture specific binding partner, or (2) detection agent or detection specific binding partner through a binding reaction. The linking agent enables the indirect binding of the capture agent to a solid phase material before the performance of the assay or during the performance of the assay. For examples, the solid phase can be plastic, derivatized plastic, magnetic, or non-magnetic metal, glass or silicon, including, for example, a test tube, microtiter well, sheet, bead, microparticle, chip, and other configurations known to those of ordinary skill in the art.

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

“Specific binding partner” or “Specific binding member”, as used interchangeably herein, is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

“Subject” and “patient” as used herein interchangeably refers to any vertebrate, including, but not limited to, a mammal (e.g., a bear, cow, cattle, pig, camel, llama, horse, goat, rabbit, sheep, hamster, guinea pig, cat, tiger, lion, cheetah, jaguar, bobcat, mountain lion, dog, wolf, coyote, rat, mouse, and a non-human primate (for example, a monkey, such as a cynomolgous or rhesus monkey, chimpanzee, etc.) and a human). In some embodiments, the subject may be a human, a non-human primate or a cat. In some embodiments, the subject is a human. The subject or patient may be undergoing other forms of treatment. In some embodiments, the subject is a human that may be undergoing other forms of treatment. In some embodiments, the subject is suspected to have, have had or has been exposed to a subject that has had or tested positive for infection with a coronavirus, such as a β-coronavirus. In other embodiments, the subject is completely asymptomatic and does not exhibit any symptoms of a coronavirus, such as a β-coronavirus, and may or may not have been exposed to a subject that has or has been exposed or infected with a coronavirus, such as a β-coronavirus.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods.

As used herein, the term “test strip” can include one or more bibulous or non-bibulous materials. If a test strip comprises more than one material, the one or more materials are preferably in fluid communication. One material of a test strip may be overlaid on another material of the test strip, such as for example, filter paper overlaid on nitrocellulose. Alternatively or in addition, a test strip may include a region comprising one or more materials followed by a region comprising one or more different materials. In this case, the regions are in fluid communication and may or may not partially overlap one another. Suitable materials for test strips include, but are not limited to, materials derived from cellulose, such as filter paper, chromatographic paper, nitrocellulose, and cellulose acetate, as well as materials made of glass fibers, nylon, dacron, PVC, polyacrylamide, cross-linked dextran, agarose, polyacrylate, ceramic materials, and the like. The material or materials of the test strip may optionally be treated to modify their capillary flow characteristics or the characteristics of the applied sample. For example, the sample application region of the test strip may be treated with buffers to correct the pH, salt concentration, or specific gravity of an applied sample to optimize test conditions.

The material or materials can be a single structure such as a sheet cut into strips or it can be several strips or particulate material bound to a support or solid surface such as found, for example, in thin-layer chromatography and may have an absorbent pad either as an integral part or in liquid contact. The material can also be a sheet having lanes thereon, capable of spotting to induce lane formation, wherein a separate assay can be conducted in each lane. The material can have a rectangular, circular, oval, triangular, or other shape provided that there is at least one direction of traversal of a test solution by capillary migration. Other directions of traversal may occur such as in an oval or circular piece contacted in the center with the test solution. However, the main consideration is that there be at least one direction of flow to a predetermined site.

The support for the test strip, where a support is desired or necessary, will normally be water insoluble, frequently non-porous and rigid but may be elastic, usually hydrophobic, and porous and usually will be of the same length and width as the strip but may be larger or smaller. The support material can be transparent, and, when a test device of the present technology is assembled, a transparent support material can be on the side of the test strip that can be viewed by the user, such that the transparent support material forms a protective layer over the test strip where it may be exposed to the external environment, such as by an aperture in the front of a test device. A wide variety of non-mobilizable and non-mobilizable materials, both natural and synthetic, and combinations thereof, may be employed provided only that the support does not interfere with the capillary action of the material or materials, or non-specifically bind assay components, or interfere with the signal producing system. Illustrative polymers include polyethylene, polypropylene, poly(4-methylbutene), polystyrene, polymethacrylate, poly(ethylene terephthalate), nylon, poly(vinyl butyrate), glass, ceramics, metals, and the like. Elastic supports may be made of polyurethane, neoprene, latex, silicone rubber and the like.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

“Variant” is used herein to describe a peptide or polypeptide that differs from a reference peptide or polypeptide in amino acid sequence by the insertion, deletion, or conservative substitution of amino acids, but retains at least one biological activity. Representative examples of “biological activity” include the ability to be bound by a specific antigen or antibody, or to promote an immune response. Variant is also used herein to describe a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to refer to an antigenically-reactive fragment of an anti-analyte antibody that differs from the corresponding fragment of anti-analyte antibody in amino acid sequence but is still antigenically reactive and can compete with the corresponding fragment of anti-analyte antibody for binding with the analyte. “Variant” also can be used to describe a polypeptide or a fragment thereof that has been differentially processed, such as by proteolysis, phosphorylation, or other post-translational modification, yet retains its antigen reactivity.

“Vector” is used herein to describe a nucleic acid molecule that can transport another nucleic acid to which it has been linked. One type of vector is a “plasmid”, which refers to a circular double-stranded DNA loop into which additional DNA segments may be ligated. Another type of vector is a viral vector, wherein additional DNA segments may be ligated into the viral genome. Certain vectors can replicate autonomously in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. “Plasmid” and “vector” may be used interchangeably as the plasmid is the most commonly used form of vector. However, other forms of expression vectors, such as viral vectors (e.g., replication defective retroviruses, adenoviruses and adeno-associated viruses), which serve equivalent functions, can be used. In this regard, RNA versions of vectors (including RNA viral vectors) may also find use in the context of the present disclosure/

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. METHODS FOR DETECTING THE PRESENCE OF OR DETERMINING THE AMOUNT OF SARS-COV-2 PROTEIN IN A BIOLOGICAL SAMPLE FROM A SUBJECT

The present disclosure relates to methods for (a) detecting the presence of at least one SARS-CoV-2 protein, such as at least one SARS-CoV-2 nucleocapsid protein; or (b) determining or measuring the amount or level of at least one SARS-CoV-2 protein, such as at least one SARS-CoV-2 nucleocapsid protein, in one or more biological samples obtained from one or more subjects. In still yet other aspects, the methods described herein can be used as an aid in the diagnosis of a SARS-CoV-2 infection. For example, the methods described herein can be used in conjunction with clinical presentation and other laboratory tests to aid in the diagnosis of SARS-CoV-2 infection in a subject (e.g., who may or may not exhibit signs and/or symptoms of infection, or be suspected of having SARS-CoV-2).

In some aspects, the detection in samples of at least one SARS-CoV-2 nucleocapsid protein (e.g., antigen) signals a reaction to SARS-CoV-2, and thus the current or past presence in the subject of the virus. In one aspect, the methods relate to (a) detecting the presence of at least one SARS-CoV-2 nucleocapsid protein (e.g., antigen); or (b) determining or measuring the amount, level or concentration of at least one SARS-CoV-2 nucleocapsid protein (e.g., antigen) in one or more biological samples obtained from one or more subjects (e.g., who may or may not exhibit signs and/or symptoms of infection and suspected of having SARS-CoV-2). In one aspect, the methods relate to detecting the presence of at least one SARS-CoV-2 nucleocapsid protein in one or more biological samples obtained from a subject (e.g., such as a human, a non-human primate, a cat, etc.). In another aspect, the methods relate to determining or measuring the amount, level or concentration of at least one SARS-CoV-2 nucleocapsid protein, in one or more biological samples obtained from a subject (e.g., such as a human, a non-human primate, a cat, etc.). It should be understood that a “negative” result obtained using the methods described herein (e.g., where the presence of at least one SARS-CoV-2 nucleocapsid protein is not detected and/or the amount, level or concentration of at least one SARS-CoV-2 nucleocapsid protein cannot be determined or is below a predetermined level or cutoff) does not rule out prior or current infection with SARS-CoV-2, particularly in those subjects who have been in contact with the virus (e.g., health care workers). Typically such subjects might receive follow-up or further testing with a molecular diagnostic to further rule out infection in said individuals.

In some embodiments, detecting the presence of or measuring the amount, level or concentration of at least SARS-CoV-2 nucleocapsid protein includes contacting the sample, either simultaneously or sequentially, in any order, with: (1) at least one first specific binding partner which specifically binds to at least one SARS-CoV-2 nucleocapsid protein to form at least one first specific binding partner-SARS-CoV-2 nucleocapsid complex; (2) at least one second specific binding partner comprising at least one detectable label (e.g., detection reagent or conjugate) that specifically binds to the SARS-CoV-2 nucleocapsid complex at a different location (e.g., epitope) than the at least one first specific binding partner such that an at least one first specific binding partner-SARS-CoV-2 nucleocapsid protein-second specific binding partner complex is formed; and (3) at least one polycation having a molecular weight of at least about 500 daltons or greater, and detecting the presence or determining or measuring the amount or concentration of at least one SARS-CoV-2 nucleocapsid protein in the sample based on the signal generated by the detectable label in the first specific binding partner-SARS-CoV-2 nucleocapsid protein-second specific binding partner complex.

In some embodiments, the at least one first specific binding partner (e.g., capture reagent) comprises at least one anti-SARS-CoV antibody, anti-SARS-CoV-2 antibody or fragment thereof that specifically binds to a first location (e.g., epitope) on the at least one SARS-CoV-2 nucleocapsid protein. In some embodiments, the at least one second specific binding partner (e.g., detection reagent or conjugate) comprises at least one anti-SARS-CoV antibody, anti-SARS-CoV-2 antibody, or fragment thereof or a recombinant antigen, that binds to a second location (e.g., epitope) on the SARS-CoV-2 nucleocapsid protein that is different than the first location of the first specific binding partner.

The “At least one First Specific Binding Partner” and the “At least one Second Specific Binding Partner”

In some aspects, the at least one first specific binding partner (e.g., capture reagent) comprises at least one anti-SARS-CoV, anti-SARS-CoV-2 antibody, or fragment thereof that specifically binds to a first location (e.g., epitope) on at least one SARS-CoV-2 nucleocapsid protein. The antibody or fragment thereof used as the at least one first specific binding partner is not critical and can be a polyclonal antibody, a monoclonal antibody, a humanized antibody, a chimeric antibody, a fully human antibody, a bispecific antibody, a multi-specific antibody, a single-chain variable fragment (“scFv”), a single chain antibody, a single domain antibody, a Fab fragment, a F(ab′) fragment, a F(ab′)₂ fragment, a disulfide-linked Fv (“sdFv”), or an anti-idiotypic (“anti-Id”) antibody, dual-domain antibody, dual variable domain (DVD) or triple variable domain (TVD) antibody. An example of a first specific binding partner that can be used in the methods described above is scFv antibody N18 described in Zhao, et al., Microbes and Infection, 9:1026-1033 (2007), the contents of which are herein incorporated by reference.

In some aspects, the at least one second specific binding partner (e.g., detection reagent or conjugate) comprises a label and (i) at least one anti-SARS-CoV, antibody, anti-SARS-CoV-2 antibody, or fragment thereof that specifically binds to a second location (e.g., epitope) on at least one SARS-CoV-2 nucleocapsid protein that is different than the first location of the at least one first specific binding partner; and/or (ii) at least one recombinant antigen.

The antibody or fragment thereof (e.g., at least one anti-SARS-CoV antibody, anti-SARS-CoV-2 antibody, or fragment thereof) used as the at least one second specific binding partner is not critical and can be a polyclonal antibody, a monoclonal antibody, a humanized antibody, a chimeric antibody, a fully human antibody, a bispecific antibody, a single-chain variable fragment (“scFv”), a single chain antibody, a single domain antibody, a Fab fragment, a F(ab′) fragment, a F(ab′)₂ fragment, a disulfide-linked Fv (“sdFv”), or an anti-idiotypic (“anti-Id”) antibody, dual-domain antibody, dual variable domain (DVD) or triple variable domain (TVD) antibody. An example of a second specific binding partner that can be used in the methods described above is human monoclonal antibody CR3009 described in van den Brink et al., Journal of Vir. 79(3):1635-1644 (February 2005), the contents of which are herein incorporated by reference.

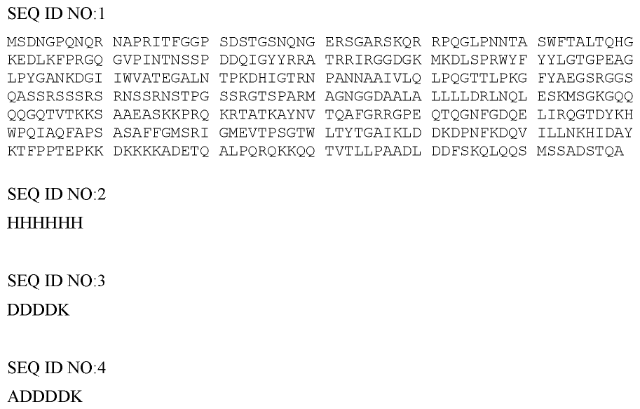

In some aspects, the at least one first specific binding partner and the at least one second specific binding partner is specific for and binds at least one recombinant antigen. The recombinant antigen comprises at least one β-coronavirus isolated polypeptide or variant thereof from a β-coronaviruses nucleocapsid protein or variant thereof. The nucleocapsid protein of β-coronaviruses comprises two separate domains (a) a N-terminal domain (NTD) or N-terminal binding domain (NBD) and (b) a C-terminal domain (CTD) or C-terminal binding domain (CBD). For example, FIG. 1 and SEQ ID NO:1 provide a nucleocapsid protein from a strain of human SARS-CoV-2. The NTD can be found in amino acids 1-209 of FIG. 1 and SEQ ID NO:1. The CTD can be found in amino acids 210-419 of FIG. 1 and SEQ ID NO:1.

In some aspects, the methods described herein can detect the presence of or measuring the amount, level or concentration of at least one isolated SARS-CoV-2 variant nucleocapsid protein. The at least one variant can be in the NTD, CTD ,or NTD and CTD domain. In some aspects, an isolated SARS-CoV-2 variant nucleocapsid protein can comprise one or more substitutions in one or more of the following amino acid positions within SEQ ID NO:1 as shown below in Table 1.

TABLE 1

| | | Strain | | | | | | | |
|---|---|---|---|---|---|---|---|---|---|
| | | Ohio | Ohio | | | | | | |
| | Country | COH.26G/ | COH.20G/ | Canada | Brazil | Brazil | USCF | UK | SA |
| Protein | Mutation | 677H | 501Y | L00311131 | P.1 | B.1.1.28 | UC301-3 | B.1.1.7 | 501Y.V2 |
| Nucleocapsid | D3L | | | | | | | | |
| Nucleocapsid | S235F | | | | | | X | X | |
| Nucleocapsid | M234I | | | X | | X | | | |
| Nucleocapsid | K373N | | | | | | X | | |
| Nucleocapsid | D377Y | X | | | | | | | |
| Nucleocapsid | A376T | | | X | | | | | |

In other aspects, the isolated SARS-CoV-2 variant nucleocapsid polypeptide can comprise one or more of substitutions and/or deletions in one or more of the following amino acid positions within SEQ ID NO:1: (1) replacing aspartic acid with leucine at amino acid position 3 (D3L); (2) replacing proline with threonine at amino acid position 13 (P6T); (3) replacing proline with leucine at amino acid position 13 (P13L); (4) replacing serine with isoleucine at amino acid position 33 (S33I); (5) replacing arginine with serine at amino acid position 92 (R92S); (6) replacing glycine with arginine at amino acid position 120 (G120R); (7) replacing leucine with phenylalanine at amino acid position 139 (L139F); (8) replacing alanine with serine at amino acid position 152 (A152S); (9) replacing alanine with serine at amino acid position 156 (A156S); (10) replacing arginine with leucine at amino acid position 191 (R191L); (11) replacing serine with leucine at amino acid position 194 (S194L); (12) replacing serine with asparagine at amino acid 202 (S202N); (13) replacing arginine with lysine at amino acid 203 position (R203K); (14) replacing glycine with arginine at amino acid position 204 (G204R); (15) replacing threonine with isoleucine at amino acid position 205 (T2051); (16) replacing methionine with isoleucine at amino acid position 234 (M234I); (17) replacing serine with phenylalanine at amino acid position 235 (S235F); (18) replacing glycine with cysteine at amino acid position 236 (G236C); (19) replacing proline with serine at amino acid position 302 (P302S); (20) replacing proline with serine at amino acid position 344 (P344S); (21) replacing aspartic acid with tyrosine at amino acid position 348 (D348Y); (22) replacing threonine with isoleucine at amino acid position 362 (T362I); (23) replacing lysine with asparagine at amino acid position 373 (K373N); (24) replacing alanine with threonine at amino acid position 376 (A376T); (25) replacing aspartic acid with tyrosine at amino acid position 377 (D377Y); (26) replacing threonine with isoleucine at amino acid position 393 (T393I); (27) replacing aspartic acid with asparagine at amino acid position 399 (D399N); or (28) any combinations of (1)-(27), either alone or combined with any other substitutions and/or deletions in SEQ ID NO:1 other than those recited in (1)-(27).

In other aspects, the isolated SARS-CoV-2 variant nucleocapsid polypeptide can comprise one or more substitutions and/or deletions in one or more positions of amino acids 210 to 419 of SEQ ID NO:1: (1) replacing methionine with isoleucine at amino acid position 234 (M234I); (2) replacing serine with phenylalanine at amino acid position 235 (S235F); (3) replacing glycine with cysteine at amino acid position 236 (G236C); (4) replacing proline with serine at amino acid position 302 (P302S); (5) replacing proline with serine at amino acid position 344 (P344S); (6) replacing aspartic acid with tyrosine at amino acid position 348 (D348Y); (7) replacing threonine with isoleucine at amino acid position 362 (T362I); (8) replacing lysine with asparagine at amino acid position 373 (K373N); (9) replacing alanine with threonine at amino acid position 376 (A376T); (10) replacing aspartic acid with tyrosine at amino acid position 377 (D377Y); (11) replacing threonine with isoleucine at amino acid position 393 (T393I); (12) replacing aspartic acid with asparagine at amino acid position 399 (D399N); or (13) any combinations of (1)-(12), either alone or combined with any other substitutions and/or deletions in amino acids 210 to 419 of SEQ ID NO:1 other than those recited in (1)-(12).

In another aspect, the nucleocapsid protein or variant thereof against which the first specific binding partner and/or the second specific binding partner is directed can have a length of about 5 amino acids to about 500 amino acids, about 10 amino acids to about 500 amino acids, about 15 amino acids to about 500 amino acids, about 20 amino acids to about 500 amino acids, about 26 amino acids to about 500 amino acids, about 30 amino acids to about 500 amino acids, about 40 amino acids to about 500 amino acids, about 50 amino acids to about 500 amino acids, about 60 amino acids to about 500 amino acids, about 70 amino acids to about 500 amino acids, about 75 amino acids to about 500 amino acids, about 80 amino acids to about 500 amino acids, about 90 amino acids to about 500 amino acids, about 100 amino acids to about 500 amino acids, about 5 amino acids to about 400 amino acids, about 10 amino acids to about 400 amino acids, about 15 amino acids to about 400 amino acids, about 20 amino acids to about 400 amino acids, about 26 amino acids to about 400 amino acids, about 30 amino acids to about 400 amino acids, about 40 amino acids to about 400 amino acids, about 50 amino acids to about 400 amino acids, about 60 amino acids to about 400 amino acids, about 70 amino acids to about 400 amino acids, about 75 amino acids to about 400 amino acids, about 80 amino acids to about 400 amino acids, about 90 amino acids to about 400 amino acids, about 100 amino acids to about 400 amino acids, about 5 amino acids to about 300 amino acids, about 10 amino acids to about 300 amino acids, about 15 amino acids to about 300 amino acids, about 20 amino acids to about 300 amino acids, about 26 amino acids to about 300 amino acids, about 30 amino acids to about 300 amino acids, about 40 amino acids to about 300 amino acids, about 50 amino acids to about 300 amino acids, about 60 amino acids to about 300 amino acids, about 70 amino acids to about 300 amino acids, about 75 amino acids to about 300 amino acids, about 80 amino acids to about 300 amino acids, about 90 amino acids to about 300 amino acids, about 100 amino acids to about 300 amino acids, about 5 amino acids to about 200 amino acids, about 10 amino acids to about 200 amino acids, about 15 amino acids to about 200 amino acids, about 20 amino acids to about 200 amino acids, about 26 amino acids to about 200 amino acids, about 30 amino acids to about 200 amino acids, about 40 amino acids to about 200 amino acids, about 50 amino acids to about 200 amino acids, about 60 amino acids to about 200 amino acids, about 70 amino acids to about 200 amino acids, about 75 amino acids to about 200 amino acids, about 80 amino acids to about 200 amino acids, about 90 amino acids to about 200 amino acids, about 100 amino acids to about 200 amino acids, about 5 amino acids to about 100 amino acids, about 10 amino acids to about 100 amino acids, about 15 amino acids to about 100 amino acids, about 20 amino acids to about 100 amino acids, about 26 amino acids to about 100 amino acids, about 30 amino acids to about 100 amino acids, about 40 amino acids to about 100 amino acids, about 50 amino acids to about 100 amino acids, about 60 amino acids to about 100 amino acids, about 70 amino acids to about 100 amino acids, about 75 amino acids to about 100 amino acids, about 80 amino acids to about 100 amino acids, or about 90 amino acids to about 100 amino acids.

In some aspects, the nucleocapsid protein against which the first specific binding partner and/or the second specific binding partner is directed comprises the amino acids 1-419 from a human SARS-CoV-2 (see, for example, SEQ ID NO:1) or a fragment or variant thereof

A “fragment” of SEQ ID NO:1 refers to a protein or polypeptide that comprises a part that is less than the entirety of SEQ ID NO:1. A fragment of SEQ ID NO:1 can comprise from about 5 to about 415 contiguous amino acids. In another aspect, a fragment of SEQ ID NO:1 comprises at least about 5 contiguous amino acids of SEQ ID NO:1, at least about 10 contiguous amino acids of SEQ ID NO:1, at least about 15 contiguous amino acids of SEQ ID NO:1, at least about 20 contiguous amino acids of SEQ ID NO:1, at least about 25 contiguous amino acids of SEQ ID NO:1, at least about 30 contiguous amino acids of SEQ ID NO:1, at least about 35 contiguous amino acids of SEQ ID NO:1, at least about 40 contiguous amino acids of SEQ ID NO:1, at least about 45 contiguous amino acids of SEQ ID NO:1, at least about 50 contiguous amino acids of SEQ ID NO:1, at least about 55 contiguous amino acids of SEQ ID NO:1, at least about 60 contiguous amino acids of SEQ ID NO:1, at least about 65 contiguous amino acids of SEQ ID NO:1, at least about 70 contiguous amino acids of SEQ ID NO:1, at least about 75 contiguous amino acids of SEQ ID NO:1, at least about 80 contiguous amino acids of SEQ ID NO:1, at least about 85 contiguous amino acids of SEQ ID NO:1, at least about 90 contiguous amino acids of SEQ ID NO:1, at least about 95 contiguous amino acids of SEQ ID NO:1, at least 100 contiguous amino acids of SEQ ID NO:1, at least about 105 contiguous amino acids of SEQ ID NO:1, at least about 110 contiguous amino acids of SEQ ID NO:1, at least about 115 contiguous amino acids of SEQ ID NO:1, at least about 120 contiguous amino acids of SEQ ID NO:1, at least about 125 contiguous amino acids of SEQ ID NO:1, at least about 130 contiguous amino acids of SEQ ID NO:1, at least about 135 contiguous amino acids of SEQ ID NO:1, at least about 140 contiguous amino acids of SEQ ID NO:1, at least about 145 contiguous amino acids of SEQ ID NO:1, at least about 150 contiguous amino acids of SEQ ID NO:1, at least about 155 contiguous amino acids of SEQ ID NO:1, at least about 160 contiguous amino acids of SEQ ID NO:1, at least about 165 contiguous amino acids of SEQ ID NO:1, at least about 170 contiguous amino acids of SEQ ID NO:1, at least about 175 contiguous amino acids of SEQ ID NO:1, at least about 180 contiguous amino acids of SEQ ID NO:1, at least about 185 contiguous amino acids of SEQ ID NO:1, at least about 190 contiguous amino acids of SEQ ID NO:1, at least about 95 contiguous amino acids of SEQ ID NO:1, at least 200 contiguous amino acids of SEQ ID NO:1, at least about 215 contiguous amino acids of SEQ ID NO:1, at least about 220 contiguous amino acids of SEQ ID NO:1, at least about 225 contiguous amino acids of SEQ ID NO:1, at least about 230 contiguous amino acids of SEQ ID NO:1, at least about 235 contiguous amino acids of SEQ ID NO:1, at least about 240 contiguous amino acids of SEQ ID NO:1, at least about 245 contiguous amino acids of SEQ ID NO:1, at least about 250 contiguous amino acids of SEQ ID NO:1, at least about 255 contiguous amino acids of SEQ ID NO:1, at least about 260 contiguous amino acids of SEQ ID NO:1, at least about 265 contiguous amino acids of SEQ ID NO:1, at least about 270 contiguous amino acids of SEQ ID NO:1, at least about 275 contiguous amino acids of SEQ ID NO:1, at least about 280 contiguous amino acids of SEQ ID NO:1, at least about 285 contiguous amino acids of SEQ ID NO:1, at least about 290 contiguous amino acids of SEQ ID NO:1, at least about 295 contiguous amino acids of SEQ ID NO:1, at least 300 contiguous amino acids of SEQ ID NO:1, at least about 305 contiguous amino acids of SEQ ID NO:1, at least about 310 contiguous amino acids of SEQ ID NO:1, at least about 315 contiguous amino acids of SEQ ID NO:1, at least about 320 contiguous amino acids of SEQ ID NO:1, at least about 325 contiguous amino acids of SEQ ID NO:1, at least about 330 contiguous amino acids of SEQ ID NO:1, at least about 335 contiguous amino acids of SEQ ID NO:1, at least about 340 contiguous amino acids of SEQ ID NO:1, at least about 345 contiguous amino acids of SEQ ID NO:1, at least about 350 contiguous amino acids of SEQ ID NO:1, at least about 355 contiguous amino acids of SEQ ID NO:1, at least about 360 contiguous amino acids of SEQ ID NO:1, at least about 365 contiguous amino acids of SEQ ID NO:1, at least about 370 contiguous amino acids of SEQ ID NO:1, at least about 375 contiguous amino acids of SEQ ID NO:1, at least about 380 contiguous amino acids of SEQ ID NO:1, at least about 385 contiguous amino acids of SEQ ID NO:1, at least about 390 contiguous amino acids of SEQ ID NO:1, at least about 395 contiguous amino acids of SEQ ID NO:1, at least 400 contiguous amino acids of SEQ ID NO:1, at least about 405 contiguous amino acids of SEQ ID NO:1, at least 410 contiguous amino acids of SEQ ID NO:1, or at least about 415 contiguous amino acids of SEQ ID NO:1.

In some aspects, the nucleocapsid protein against which the first specific binding partner and/or the second specific binding partner is directed comprises the NTD of a nucleocapsid protein of a β-coronavirus (e.g., SARS-CoV or SARS-CoV-2) or any fragments or variants thereof. In other aspects, the nucleocapsid protein comprises amino acids 1-209 of the nucleocapsid protein of a β-coronavirus, such as, for example, SARS-CoV or SARS-CoV-2, or any fragments or variants thereof. In yet another aspect, the nucleocapsid protein comprises amino acids 1-209 from a human SARS-CoV-2 (See, for example, SEQ ID NO:1).

In some aspects, the nucleocapsid protein against which the first specific binding partner and/or the second specific binding partner is directed comprises the CTD of a nucleocapsid protein of a β-coronavirus (e.g., SARS-CoV or SARS-CoV-2) or any fragments or variants thereof. In other aspects, the nucleocapsid protein comprises amino acids 210-419 of the nucleocapsid protein of a β-coronavirus, such as, for example, SARS-CoV or SARS-CoV-2, or any fragments or variants thereof. In yet another aspect, the nucleocapsid protein comprises amino acids 210-419 from a human SARS-CoV-2 (See, for example, SEQ ID NO:1).

In some aspects, when the method uses only a first specific binding partner and a second specific binding partner, either specific binding partner can be immobilized on a solid support. For example in some aspects, the first specific binding partner can be immobilized on the solid support. In other aspects, when more than two specific binding partners are used in the method (e.g., a first specific binding partner, a second specific binding partner, a third specific binding partner, a fourth specific binding partner), any or all of the binding members can be immobilized on solid support). In some aspects, the at least one first specific binding partner and the at least one second specific binding partner may be immobilized on the same solid support. In other aspects, the at least one first specific binding partner and the at least one second specific binding partner may be immobilized on different solid supports. In other aspects, when the at least one first specific binding partner and at least one second specific binding partner are immobilized on the same solid support, the amount or ratio of at least one first specific binding partner and at least one second specific binding partner can be optimized and at least one SARS-CoV-2 nucleocapsid protein to be detected or determined (e.g., amount or concentration determined). In some aspects, when a third specific binding partner and fourth specific binding partner are used as detection agents or conjugates, the detectable label used for the third specific binding partner and the fourth specific binding partner can be the same label or can be a different label. Still further, in yet further aspects, the third specific binding partner and the fourth specific binding partner can each be at least one one anti-SARS-CoV antibody, anti-SARS-CoV-2 antibody or fragment thereof.

At Least One Polycation having a Molecular Weight of at Least about 500 Daltons or greater

In some aspects, the at least one polycation has a molecular weight of about 500 daltons or greater. In yet other aspects, the at least one polycation has a molecular weight of about 800 daltons or greater. In still other aspects, the at least one polycation has a molecular weight of about 1,000 daltons or greater. In still yet further aspects, the at least one polycation has a molecular weight of about 2,000 daltons or greater. In still yet other aspects, the at least one polycation has a molecular weight of about 3,000 daltons or greater, about 4,000 daltons or greater, about 5,000 daltons or greater, about 6,000 daltons or greater, about 7,000 daltons or greater, about 8,000 daltons or greater, about 9,000 daltons or greater, about 10,000 daltons or greater, about 15,000 daltons or greater, about 20,000 daltons or greater, about 25,000 daltons or greater, about 30,000 daltons or greater, about 35,000 daltons or greater, about 40,000 daltons or greater, about 45,000 daltons or greater, about 50,000 daltons or greater, about 55,000 daltons or greater, about 60,000 daltons or greater, about 65,000 daltons or greater, about 70,000 daltons or greater, about 75,000 daltons or greater, about 80,000 daltons or greater, about 85,000 daltons or greater, about 90,000 daltons or greater, about 100,000 daltons or greater, about 150,000 daltons or greater, about 200,000 daltons or greater, about 250,000 daltons or greater, about 300,000 or greater, about 350,000 daltons or greater, about 400,000 daltons or greater, about 450,000 daltons or greater, or about 500,000 daltons or greater.

In some aspects, the at least one polycation is at least one polylysine (such as poly-L-lysines and poly-D-lysines) having a molecular weight range of about 1000 daltons to about 400,000 daltons. In other aspects, the at least one polylysine has a molecular weight range of about 1000 daltons to about 100,000 daltons. In yet another aspect, the at least one polylysine has a molecular weight range of about 1000 daltons to about 50,000 daltons. In still yet another aspect, the at least one polylysine has a molecular weight range of about 1000 daltons to about 25,000 daltons. In yet still a further aspect, the at least one one polylysine has a molecular weight range of about 1000 daltons to about 15,000 daltons. In still yet another aspect, the at least one polylysine has a molecular weight range of about 1000 daltons to about 10,000 daltons. In still yet a further aspect, the at least one polylysine has a molecular weight range of about 1000 daltons to about 5,000 daltons. Examples of polylysines that can be used include poly-L-lysine hydrobromide, poly-D-lysine hydrobromide, poly-L-lysine hydrochloride, poly-L-lysine trifluoroacetate, poly(lysine,alanine) 3:1 hydrobromide, poly(lysine, arginine) 2:1 hydrobromide, poly(lysine, alanine) 1:1 hydrobromide, or poly(lysine, tryptophan) 1:4 hydrobromide.

In some aspects, the at least one polycation is at least one polyornithine (such as poly-L-ornithines or poly-DL-ornithines) having a molecular weight range of about 5000 daltons to about 500,000 daltons. Examples of polyornithines that can be used include poly-L-ornithine hydrobromide or poly-DL-ornithine hydrobromide.

In some aspects, the at least one polycation is at least one poly-L-histidine having a molecular weight range of about 5000 daltons to about 100,000 daltons. Examples of poly-L-histidines that can be used include poly-L-histidine hydrobromide.

In yet other aspects, the at least one polycation is at least one poly-L-arginine having a molecular weight range of about 5000 daltons to about 100,000 daltons. Examples of poly-L-arginines that can be used include poly-L-arginine hydrochloride and poly-L-arginine hydrobromide.

In still yet other aspects, the at least one polycation is at least one polyethylenimines having a molecular weight range of about 800 daltons to about 5,000 daltons.

In still yet further aspects, the at least one polycation is at least one DEAE-Dextran having a molecular weight range of about 500 daltons to about 500,000 daltons.

In yet other aspects, the at least one polycation is a combination of one or more of at least one polylysine, at least one polyornithine, at least one poly-L-histidine, at least one poly-L-arginine, or at least one polyethlenimines.

The amount of at least one polycation that can be used in the methods, kits and systems described herein is from about 0.1 to about 500 ng/mL. In yet another aspect, the amount of at least one polycation that can be used is from about 0.1 to about 250 ng/mL. In still yet another aspect, the amount of polycation that can be used is from about 0.1 to about 150 ng/mL. In yet still another aspect, the amount of at least one polycation that can be used is from about 0.1 to about 100 ng/mL. In still yet another aspect, the amount of at least one polycation that can be used is from about 0.1 to about 50 ng/mL. In still yet another aspect, the amount of at least one polycation that can be used is from about 0.1 to about 25 ng/mL. In yet another aspect, the amount of at least one polycation that can be used is from about 0.5 to about 250 ng/mL. In still yet another aspect, the amount of polycation that can be used is from about 0.5 to about 150 ng/mL. In yet still another aspect, the amount of at least one polycation that can be used is from about 0.5 to about 100 ng/mL. In still yet another aspect, the amount of at least one polycation that can be used is from about 0.5 to about 50 ng/mL. In still yet another aspect, the amount of at least one polycation that can be used is from about 0.5 to about 25 ng/mL. In yet another aspect, the amount of at least one polycation that can be used is from about 1 to about 250 ng/mL. In still yet another aspect, the amount of polycation that can be used is from about 1 to about 150 ng/mL. In yet still another aspect, the amount of at least one polycation that can be used is from about 1 to about 100 ng/mL. In still yet another aspect, the amount of at least one polycation that can be used is from about 1 to about 50 ng/mL. In still yet another aspect, the amount of at least one polycation that can be used is from about 1 to about 25 ng/mL. In yet another aspect, the amount of at least one polycation that can be used is from about 5 to about 250 ng/mL. In still yet another aspect, the amount of polycation that can be used is from about 5 to about 150 ng/mL. In yet still another aspect, the amount of at least one polycation that can be used is from about 5 to about 100 ng/mL. In still yet another aspect, the amount of at least one polycation that can be used is from about 5 to about 50 ng/mL. In still yet another aspect, the amount of at least one polycation that can be used is from about 5 to about 25 ng/mL. In yet another aspect, the amount of at least one polycation that can be used is from about 10 to about 250 ng/mL. In still yet another aspect, the amount of polycation that can be used is from about 10 to about 150 ng/mL. In yet still another aspect, the amount of at least one polycation that can be used is from about 10 to about 100 ng/mL. In still yet another aspect, the amount of at least one polycation that can be used is from about 10 to about 50 ng/mL. In still yet another aspect, the amount of at least one polycation that can be used is from about 10 to about 25 ng/mL. In yet still another aspect, the amount of at least one polycation that can be used is from about 1 ng/mL to about 10 ng/mL. In yet still another aspect, the amount of at least one polycation that can be used is from about 1 ng/mL to about 5 ng/mL. In yet still a further aspect, the amount of at least one polycation is about 1 ng/mL. In another aspect, the amount of at least one polycation is about 5 ng/mL. In still yet another aspect, the amount of at least one polycation is about 10 ng/mL. In still yet another aspect, the amount of at least one polycation is about 50 ng/mL. In yet still another aspect, the amount of at least one polycation is about 100 ng/mL.

It still yet other aspects, the time at which the at least one polycation is added during the performance of the method is not critical. For example, in some aspects, the at least one polycation may be added to the biological sample before the addition of either the at least one first specific binding partner or at least one second specific binding partner. In other aspects, the at least one polycation may be added simultaneously or sequentially with the at least one first specific binding partner or the at least one second specific binding partner. In still yet other aspects, the at least one polycation may be added after the addition of the at least one second specific binding partner. The inclusion or addition in the assay methods of at least one polycation as described herein has been found to improve the sensitivity of detection of the methods described above. Specifically, an improvement in sensitivity can be measured by determining the signal to noise (S/N) ratio of the methods described above with and without the addition of at least one polycation. Methods employing at least one polycation have a higher S/N ratio than methods that do not employ at least one polycation, and thus exhibit or demonstrate higher sensitivity. In some aspects, the addition of at least one polycation results in an improvement in the sensitivity (namely, a higher S/N ratio) in the above described methods of at least about 5%, at least about 10%, at least about 15%, at least about 20%, at least about 25%, at least about 30%, at least about 35%, at least about 40%, at least about 45%, at least about 55%, at least about 60%, at least about 70%, at least about 75%, at least about 80%, at least about 85%, at least about 95%, at least about 100%, at least about 110%, at least about 120%, at least about 125%, at least about 130%, at least about 140%, at least about 150%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% when compared with methods or assays that do not employ at least one polycation.

In some aspects, the improvement in sensitivity is at least about 5% to about 300% when compared with methods or assays that do not employ at least one polycation. In some other aspects, the improvement in sensitivity is at least about 10% to about 300% when compared with methods or assays that do not employ at least one polycation. In yet other aspects, the improvement in sensitivity is at least about 15% to about 50% when compared with methods or assays that do not employ at least one polycation. In yet other aspects, the improvement in sensitivity is at least about 20% to about 300% when compared with methods or assays that do not employ at least one polycation. In still other aspects, the improvement in sensitivity is at least about 25% to about 300% when compared with methods or assays that do not employ at least one polycation. In still other aspects, the improvement in sensitivity is at least about 30% to about 300% when compared with methods or assays that do not employ at least one polycation. In some aspects, the improvement in sensitivity is at least about 5% to about 200% when compared with methods or assays that do not employ at least one polycation.

In some other aspects, the improvement in sensitivity is at least about 10% to about 200% when compared with methods or assays that do not employ at least one polycation. In yet other aspects, the improvement in sensitivity is at least about 15% to about 50% when compared with methods or assays that do not employ at least one polycation. In yet other aspects, the improvement in sensitivity is at least about 20% to about 200% when compared with methods or assays that do not employ at least one polycation. In still other aspects, the improvement in sensitivity is at least about 25% to about 200% when compared with methods or assays that do not employ at least one polycation. In still other aspects, the improvement in sensitivity is at least about 30% to about 200% when compared with methods or assays that do not employ at least one polycation. In some aspects, the improvement in sensitivity is at least about 5% to about 100% when compared with methods or assays that do not employ at least one polycation. In some other aspects, the improvement in sensitivity is at least about 10% to about 100% when compared with methods or assays that do not employ at least one polycation. In yet other aspects, the improvement in sensitivity is at least about 15% to about 100% when compared with methods or assays that do not employ at least one polycation. In yet other aspects, the improvement in sensitivity is at least about 20% to about 50% when compared with methods or assays that do not employ at least one polycation. In still other aspects, the improvement in sensitivity is at least about 25% to about 100% when compared with methods or assays that do not employ at least one polycation. In still other aspects, the improvement in sensitivity is at least about 30% to about 100% when compared with methods or assays that do not employ at least one polycation.

In some aspects, the improvement in sensitivity is at least about 5% to about 50% when compared with methods or assays that do not employ at least one polycation. In some other aspects, the improvement in sensitivity is at least about 10% to about 50% when compared with methods or assays that do not employ at least one polycation. In yet other aspects, the improvement in sensitivity is at least about 15% to about 50% when compared with methods or assays that do not employ at least one polycation. In yet other aspects, the improvement in sensitivity is at least about 20% to about 50% when compared with methods or assays that do not employ at least one polycation. In still other aspects, the improvement in sensitivity is at least about 25% to about 50% when compared with methods or assays that do not employ at least one polycation. In still other aspects, the improvement in sensitivity is at least about 30% to about 50% when compared with methods or assays that do not employ at least one polycation. In some aspects, the improvement in sensitivity is at least about 5% to about 40% when compared with methods or assays that do not employ at least one polycation. In some other aspects, the improvement in sensitivity is at least about 10% to about 40% when compared with methods or assays that do not employ at least one polycation. In yet other aspects, the improvement in sensitivity is at least about 15% to about 40% when compared with methods or assays that do not employ at least one polycation. In yet other aspects, the improvement in sensitivity is at least about 20% to about 40% when compared with methods or assays that do not employ at least one polycation. In still other aspects, the improvement in sensitivity is at least about 25% to about 40% when compared with methods or assays that do not employ at least one polycation. In still other aspects, the improvement in sensitivity is at least about 30% to about 40% when compared with methods or assays that do not employ at least one polycation. In some embodiments, the biological sample is diluted or undiluted. The sample can be from about 1 to about 25 microliters, about 1 to about 24 microliters, about 1 to about 23 microliters, about 1 to about 22 microliters, about 1 to about 21 microliters, about 1 to about 20 microliters, about 1 to about 18 microliters, about 1 to about 17 microliters, about 1 to about 16 microliters, about 15 microliters or about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters or about 25 microliters. In some embodiments, the sample is from about 1 to about 150 microliters or less or from about 1 to about 25 microliters or less.

Other methods of detection include the use of or can be adapted for use on a nanopore device or nanowell device, e.g., for single molecule detection. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety. Other devices and methods appropriate for single molecule detection also can be employed.

The nature of methods described herein is not critical and the test can be any assay known in the art such as, for example, immunoassays, lateral flow assays, protein immunoprecipitation, immunoelectrophoresis, chemical analysis, SDS-PAGE and Western blot analysis, or protein immunostaining, electrophoresis analysis, a protein assay, a competitive binding assay, a lateral flow assay, a functional protein assay, or chromatography or spectrometry methods, such as high-performance liquid chromatography (HPLC) or liquid chromatography-mass spectrometry (LC/MS). Also, the assay can be employed in a clinical chemistry format such as would be known by one of ordinary skill in the art. Such assays are described in further detail herein in Sections 5-8. It is known in the art that the values (e.g., reference levels, cutoffs, thresholds, specificities, sensitivities, concentrations of calibrators and/or controls etc.) used in an assay that employs specific sample type (e.g., such as an immunoassay that utilizes serum or a point-of-care device that employs whole blood) can be extrapolated to other assay formats using known techniques in the art, such as assay standardization. For example, one way in which assay standardization can be performed is by applying a factor to the calibrator employed in the assay to make the sample concentration read higher or lower to get a slope that aligns with the comparator method. Other methods of standardizing results obtained on one assay to another assay are well known and have been described in the literature (See, for example, David Wild, Immunoassay Handbook, 4^{th} edition, chapter 3.5, pages 315-322, the contents of which are herein incorporated by reference).

### 3. TREATMENT AND MONITORING OF SUBJECTS IDENTIFIED AS HAVING SARS-COV-2

A subject identified according to the methods described above as having at least one SARS-CoV-2 nucleocapsid protein and/or having a certain amount, concentration and/or level of at least one SARS-CoV-2 nucleocapsid protein may be treated, monitored (e.g., by monitoring SARS-CoV-2 nucleocapsid protein levels and/or anti-SARS-CoV-2 IgG and/or IgM antibody levels in the subject), treated and monitored and/or monitored and treated using routine techniques known in the art. In some aspects, the methods described herein further include treating the subject (e.g., such as a human) identified as having at least one SARS-CoV-2 nucleocapsid protein and/or having a certain amount, concentration and/or level of at least one SARS-CoV-2 nucleocapsid protein in one or more biological samples obtained from the subject.

The treatment can take a variety of forms depending on whether or not the subject is asymptomatic or experiencing mild, moderate or severe infection with SARS-CoV-2. For example, subjects experiencing mild infection with SARS-CoV-2, will experience a fever, cough (with or without sputum production), anorexia, malaise, muscle pain, sore throat, dyspnea, nasal congestion, headache, diarrhea, nausea, and vomiting or any combination thereof. Subjects experiencing a moderate infection will experience a fever greater than 100.4° F. that lasts for several days, chills, shortness of breath, lethargy, or any combination thereof. Such subjects may be suffering from pneumonia. Subjects experiencing severe infection will experience trouble breathing, persistant pain or pressure in the chest, confusion, inability to rouse, bluish lips or face, or any combination thereof. Such subjects may be suffering from severe pneumonia.

If the subject is asymptomatic or has mild symptoms, the subject may be treated with rest and sleep, by keeping warm, ingesting fluids (e.g., remaining hydrated) minimizing social interaction with other subjects (e.g., remain isolated or quarantined, such as, for example, at home), or any combination thereof. Additionally, the subject can be monitored to see if symptoms arise and/or worsen.

Subjects with moderate or severe symptoms of infection with SARS-CoV-2, may be treated with one or more drugs, vaccines, convalescent plasma therapy (e.g., receiving plasma from blood taken from a subject that has survived an infection with SARS-CoV-2, or respiratory support or assistance (e.g., receiving supplemental oxygen through a nasal cannula, nasal progns, face mask, or non-invasive or invasive (e.g. intubation) ventilation) or combinations thereof. Examples of one or more drugs that can be used to treat a subject include, but are not limited to, remdesivir, hydroxychloroquine, chloroquine or combinations thereof. Subjects receiving any of the aforementioned treatment can also further be monitored using routine techniques known in the art.

In other aspects, a subject may be monitored prior to being treated for SARS-CoV-2. Such monitoring involves detecting, analyzing and/or interpreting changes in the subject's SARS-CoV-2 nucleocapsid protein levels and/or anti-SARS-CoV-2 IgG and/or IgM antibody levels over the course of time. For example, depending on a subject's SARS-CoV-2 nucleocapsid protein levels and/or SARS-CoV-2 IgM antibody levels, a subject may be monitored prior to receiving any treatment to gauge whether the subject's immune system is able to fight the virus on its own without any treatment intervention. During the course of the monitoring, if the subject's SARS-CoV-2 nucleocapsid protein levels and/or SARS-CoV-2 IgM antibody levels increase, treatment can be commenced. Likewise, during treatment, a subject's SARS-CoV-2 IgM and/or IgG antibody levels can be monitored. If during treatment the subject's SARS-CoV-2 IgM antibody levels remain high and SARS-CoV-2 IgG antibody levels remain low, the subject can be continued to be treated for SARS-CoV-2 and continued to monitored until such time that the subject's SARS-CoV-2 IgM antibody levels have lowered and SARS-CoV-2 IgG antibody levels increased.

### 4. PREPARATION/PRODUCTIONS METHODS FOR ANTIBODIES FOR USE AS SPECIFIC BINDING PARTNERS

a. Antibodies

Antibodies may be prepared by any of a variety of techniques, including those well known to those skilled in the art. In general, antibodies can be produced by cell culture techniques, including the generation of monoclonal antibodies via conventional techniques, or via transfection of antibody genes, heavy chains, and/or light chains into suitable bacterial or mammalian cell hosts, to allow for the production of antibodies, wherein the antibodies may be recombinant. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies include Chinese Hamster Ovary (CHO cells) (including dhfr-CHO cells, described in Urlaub and Chasin, Proc. Natl. Acad. Sci. USA, 77: 4216-4220 (1980)), used with a DHFR selectable marker, e.g., as described in Kaufman and Sharp, J. Mol. Biol., 159: 601-621 (1982), NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. In some aspects, the purification of the antibodies can be done in CHO and/or HEK cells using routine techniques known in the art.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies. In addition, bifunctional antibodies may be produced in which one heavy and one light chain are a human β-coronavirus antibody (i.e., binds to one or more epitopes on a β-coronavirus, such as SARS-CoV or SARS-CoV-2) and the other heavy and light chain are specific for an antigen other than a human β-coronavirus (e.g., such as SARS-CoV or SARS-CoV-2) by crosslinking an antibody to a second antibody by standard chemical crosslinking methods.

In a preferred system for recombinant expression of an antibody, or antigen-binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the method of synthesizing a recombinant antibody may be by culturing a host cell in a suitable culture medium until a recombinant antibody is synthesized. The method can further comprise isolating the recombinant antibody from the culture medium.

Methods of preparing monoclonal antibodies involve the preparation of immortal cell lines capable of producing antibodies having the desired specificity. Such cell lines may be produced from spleen cells obtained from an immunized animal. The animal may be immunized with β-coronavirus (e.g., such as SARS-CoV or SARS-CoV-2) or a fragment (e.g., such as from the nucleocapsid and/or spike proteins) and/or variant thereof. The peptide used to immunize the animal may comprise amino acids encoding human Fc, for example the fragment crystallizable region or tail region of human antibody. The spleen cells may then be immortalized by, for example, fusion with a myeloma cell fusion partner. A variety of fusion techniques may be employed. For example, the spleen cells and myeloma cells may be combined with a nonionic detergent for a few minutes and then plated at low density on a selective medium that supports that growth of hybrid cells, but not myeloma cells. One such technique uses hypoxanthine, aminopterin, thymidine (HAT) selection. Another technique includes electrofusion. After a sufficient time, usually about 1 to 2 weeks, colonies of hybrids are observed. Single colonies are selected and their culture supernatants tested for binding activity against the polypeptide. Hybridomas having high reactivity and specificity may be used.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. Affinity chromatography is an example of a method that can be used in a process to purify the antibodies.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites.

The Fv fragment can be produced by preferential proteolytic cleavage of an IgM, and on rare occasions IgG or IgA immunoglobulin molecules. The Fv fragment may be derived using recombinant techniques. The Fv fragment includes a non-covalent VH:: VL heterodimer including an antigen-binding site that retains much of the antigen recognition and binding capabilities of the native antibody molecule.

The antibody, antibody fragment, or derivative may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, yeast or the like, display library); e.g., as available from various commercial vendors such as Cambridge Antibody Technologies (Cambridgeshire, UK), MorphoSys (Martinsreid/Planegg, Del.), Biovation (Aberdeen, Scotland, UK) Biolnvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1997) Microbiol. Immunol. 41:901-907; Sandhu et al. (1996) Crit. Rev. Biotechnol. 16:95-118; Eren et al. (1998) Immunol. 93:154-161) that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass).; Gray et al. (1995) J. Imm. Meth. 182:155-163; Kenny et al. (1995) Bio/Technol. 13:787-790); B-cell selection (Steenbakkers et al. (1994) Molec. Biol. Reports 19:125-134 (1994)).

An affinity matured antibody may be produced by any one of a number of procedures that are known in the art. For example, see Marks et al., BioTechnology, 10: 779-783 (1992) describes affinity maturation by VH and VL domain shuffling. Random mutagenesis of CDR and/or framework residues is described by Barbas et al., Proc. Nat. Acad. Sci. USA, 91: 3809-3813 (1994); Schier et al., Gene, 169: 147-155 (1995); Yelton et al., J. Immunol., 155: 1994-2004 (1995); Jackson et al., J. Immunol., 154(7): 3310-3319 (1995); Hawkins et al., J. Mol. Biol., 226: 889-896 (1992). Selective mutation at selective mutagenesis positions and at contact or hypermutation positions with an activity enhancing amino acid residue is described in U.S. Pat. No. 6,914,128 B1.

Antibody variants can also be prepared using delivering a polynucleotide encoding an antibody to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

Antibody variants also can be prepared by delivering a polynucleotide to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbiol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al., Adv. Exp. Med Biol. (1999) 464:127-147 and references cited therein. Antibody variants have also been produced in large amounts from transgenic plant seeds including antibody fragments, such as single chain antibodies (scFv's), including tobacco seeds and potato tubers. See, e.g., Conrad et al. (1998) Plant Mol. Biol. 38:101-109 and reference cited therein. Thus, antibodies can also be produced using transgenic plants, according to known methods.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

Small antibody fragments may be diabodies having two antigen-binding sites, wherein fragments comprise a heavy chain variable domain (VH) connected to a light chain variable domain (VL) in the same polypeptide chain (VH VL). See for example, EP 404,097; WO 93/11161; and Hollinger et al., (1993) Proc. Natl. Acad. Sci. USA 90:6444-6448. By using a linker that is too short to allow pairing between the two domains on the same chain, the domains are forced to pair with the complementary domains of another chain and create two antigen-binding sites. See also, U.S. Pat. No. 6,632,926 to Chen et al. which is hereby incorporated by reference in its entirety and discloses antibody variants that have one or more amino acids inserted into a hypervariable region of the parent antibody and a binding affinity for a target antigen which is at least about two fold stronger than the binding affinity of the parent antibody for the antigen.

The antibody may be a linear antibody. The procedure for making a linear antibody is known in the art and described in Zapata et al., (1995) Protein Eng. 8(10):1057-1062. Briefly, these antibodies comprise a pair of tandem Fd segments (VH-CH1-VH-CH1) which form a pair of antigen binding regions. Linear antibodies can be bispecific or monospecific.

The antibodies may be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

It may be useful to detectably label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (131I) yttrium-90 (90Y), bismuth-212 (212Bi), bismuth-213 (213Bi), technetium-99m (99mTc), rhenium-186 (186Re), and rhenium-188 (188Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naj a naj a atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; 1y207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

Antibody production via the use of hybridoma technology, the selected lymphocyte antibody method (SLAM), transgenic animals, and recombinant antibody libraries is described in more detail below.

Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, second edition, (Cold Spring Harbor Laboratory Press, Cold Spring Harbor, 1988); Hammerling, et al., In Monoclonal Antibodies and T-Cell Hybridomas, (Elsevier, N.Y., 1981). It is also noted that the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods of generating monoclonal antibodies as well as antibodies produced by the method may comprise culturing a hybridoma cell secreting an antibody wherein, preferably, the hybridoma is generated by fusing splenocytes isolated from an animal, e.g., a rat or a mouse, immunized with a β-coronavirus, such as SARS-CoV or SARS-CoV-2 (e.g., such as a human, mouse, rat, rabbit SARS-CoVor SARS-CoV-2), or a fragment or variant thereof (collectively referred to as a “β-coronavirus antigen”) with myeloma cells and then screening the hybridomas resulting from the fusion for hybridoma clones that secrete an antibody able to bind a polypeptide. Briefly, rats can be immunized with a β-coronavirus antigen. In a preferred embodiment, the β-coronavirus antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks; however, a single administration of the polypeptide may also be used.

After immunization of an animal with a β-coronavirus antigen, antibodies and/or antibody-producing cells may be obtained from the animal. An anti-β-coronavirus antibody-containing serum is obtained from the animal by bleeding or sacrificing the animal. The serum may be used as it is obtained from the animal, an immunoglobulin fraction may be obtained from the serum, or the anti-β-coronavirus antibodies may be purified from the serum. Serum or immunoglobulins obtained in this manner are polyclonal, thus having a heterogeneous array of properties.

Once an immune response is detected, e.g., antibodies specific for the antigen β-coronavirus are detected in the rat serum, the rat spleen is harvested and splenocytes isolated. The splenocytes are then fused by well-known techniques to any suitable myeloma cells, for example, cells from cell line SP20 available from the American Type Culture Collection (ATCC, Manassas, Va., US). Hybridomas are selected and cloned by limited dilution. The hybridoma clones are then assayed by methods known in the art for cells that secrete antibodies capable of binding to a β-coronavirus (such as, for example, SARS-CoV or SARS-CoV-2). Ascites fluid, which generally contains high levels of antibodies, can be generated by immunizing rats with positive hybridoma clones.

In another embodiment, antibody-producing immortalized hybridomas may be prepared from the immunized animal. After immunization, the animal is sacrificed and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using a β-coronavirus, or a portion thereof, or a cell expressing a β-coronavirus or portion thereof. In a preferred embodiment, the initial screening is performed using an enzyme-linked immunosorbent assay (ELISA) or a radioimmunoassay (MA), preferably an ELISA. An example of ELISA screening is provided in PCT Publication No. WO 00/37504.

β-coronavirus antibody-producing hybridomas are selected, cloned, and further screened for desirable characteristics, including robust hybridoma growth, high antibody production, and desirable antibody characteristics. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, hybridomas are rat hybridomas. In another embodiment, hybridomas are produced in a non-human, non-rat species such as mice, sheep, pigs, goats, cattle, or horses. In yet another preferred embodiment, the hybridomas are human hybridomas, in which a human non-secretory myeloma is fused with a human cell expressing an anti-β-coronavirus antibody.

Antibody fragments that recognize specific epitopes may be generated by known techniques. For example, Fab and F(ab′)₂ fragments may be produced by proteolytic cleavage of immunoglobulin molecules, using enzymes such as papain (to produce two identical Fab fragments) or pepsin (to produce an F(ab′)₂ fragment). A F(ab′)₂ fragment of an IgG molecule retains the two antigen-binding sites of the larger (“parent”) IgG molecule, including both light chains (containing the variable light chain and constant light chain regions), the CH1 domains of the heavy chains, and a disulfide-forming hinge region of the parent IgG molecule. Accordingly, an F(ab′)₂ fragment is still capable of crosslinking antigen molecules like the parent IgG molecule.

Monoclonal Antibodies Using SLAM

In another aspect, recombinant antibodies are generated from single, isolated lymphocytes using a procedure referred to in the art as the selected lymphocyte antibody method (SLAM), as described in U.S. Pat. No. 5,627,052; PCT Publication No. WO 92/02551; and Babcook et al., Proc. Natl. Acad. Sci. USA, 93: 7843-7848 (1996). In this method, single cells secreting antibodies of interest, e.g., lymphocytes derived from any one of the immunized animals are screened using an antigen-specific hemolytic plaque assay, wherein the β-coronavirus antigen is coupled to sheep red blood cells using a linker, such as biotin, and used to identify single cells that secrete antibodies with specificity for a β-coronavirus. Following identification of antibody-secreting cells of interest, heavy- and light-chain variable region cDNAs are rescued from the cells by reverse transcriptase-PCR (RT-PCR) and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example, by panning the transfected cells to isolate cells expressing antibodies to a β-coronavirus. The amplified immunoglobulin sequences further can be manipulated in vitro, such as by in vitro affinity maturation method. See, for example, PCT Publication No. WO 97/29131 and PCT Publication No. WO 00/56772.

Monoclonal Antibodies Using Transgenic Animals

In another embodiment, antibodies are produced by immunizing a non-human animal comprising some, or all, of the human immunoglobulin locus with a β-coronavirus antigen. In an embodiment, the non-human animal is a XENOMOUSE® transgenic mouse, an engineered mouse strain that comprises large fragments of the human immunoglobulin loci and is deficient in mouse antibody production. See, e.g., Green et al., Nature Genetics, 7: 13-21 (1994) and U.S. Pat. Nos. 5,916,771; 5,939,598; 5,985,615; 5,998,209; 6,075,181; 6,091,001; 6,114,598; and 6,130,364. See also PCT Publication Nos. WO 91/10741; WO 94/02602; WO 96/34096; WO 96/33735; WO 98/16654; WO 98/24893; WO 98/50433; WO 99/45031; WO 99/53049; WO 00/09560; and WO 00/37504. The XENOMOUSE® transgenic mouse produces an adult-like human repertoire of fully human antibodies, and generates antigen-specific human monoclonal antibodies. The XENOMOUSE® transgenic mouse contains approximately 80% of the human antibody repertoire through introduction of megabase sized, germline configuration YAC fragments of the human heavy chain loci and x light chain loci. See Mendez et al., Nature Genetics, 15: 146-156 (1997), Green and Jakobovits, J Exp. Med., 188: 483-495 (1998), the disclosures of which are hereby incorporated by reference.

Monoclonal Antibodies Using Recombinant Antibody Libraries

In vitro methods also can be used to make the antibodies, wherein an antibody library is screened to identify an antibody having the desired β-coronavirus-binding specificity. Methods for such screening of recombinant antibody libraries are well known in the art and include methods described in, for example, U.S. Pat. No. 5,223,409 (Ladner et al.); PCT Publication No. WO 92/18619 (Kang et al.); PCT Publication No. WO 91/17271 (Dower et al.); PCT Publication No. WO 92/20791 (Winter et al.); PCT Publication No. WO 92/15679 (Markland et al.); PCT Publication No. WO 93/01288 (Breitling et al.); PCT Publication No. WO 92/01047 (McCafferty et al.); PCT Publication No. WO 92/09690 (Garrard et al.); Fuchs et al., Bio/Technology, 9: 1369-1372 (1991); Hay et al., Hum. Antibod. Hybridomas, 3: 81-85 (1992); Huse et al., Science, 246: 1275-1281 (1989); McCafferty et al., Nature, 348: 552-554 (1990); Griffiths et al., EMBO J., 12: 725-734 (1993); Hawkins et al., J. Mol. Biol., 226: 889-896 (1992); Clackson et al., Nature, 352: 624-628 (1991); Gram et al., Proc. Natl. Acad. Sci. USA, 89: 3576-3580 (1992); Garrard et al., Bio/Technology, 9: 1373-1377 (1991); Hoogenboom et al., Nucl. Acids Res., 19: 4133-4137 (1991); Barbas et al., Proc. Natl. Acad. Sci. USA, 88: 7978-7982 (1991); U.S. Patent Application Publication No. 2003/0186374; and PCT Publication No. WO 97/29131, the contents of each of which are incorporated herein by reference.

The recombinant antibody library may be from a subject immunized with a β-coronavirus antigen. Alternatively, the recombinant antibody library may be from a naive subject, i.e., one who has not been immunized with a β-coronavirus antigen, such as a human antibody library from a human subject who has not been immunized with a human β-coronavirus antigen. Antibodies are selected by screening the recombinant antibody library with the peptide comprising human β-coronavirus or fragment thereof to thereby select those antibodies that recognize the β-coronavirus (e.g., SARS-CoV or SARS-CoV-2) of interest. Methods for conducting such screening and selection are well known in the art, such as described in the references in the preceding paragraph. To select antibodies having particular binding affinities, the art-known method of surface plasmon resonance can be used to select antibodies having the desired K_{off} rate constant. To select antibodies having a particular neutralizing activity for a β-coronavirus, such as SARS-CoV or SARS-CoV-2, such as those with a particular IC₅₀, standard methods known in the art for assessing the inhibition of β-coronavirus activity may be used.

For example, antibodies can also be generated using various phage display methods known in the art. In phage display methods, functional antibody domains are displayed on the surface of phage particles which carry the polynucleotide sequences encoding them. Such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv, or disulfide stabilized Fv antibody domains recombinantly fused to either the phage gene III or gene VIII protein. Examples of phage display methods that can be used to make the antibodies include those disclosed in Brinkmann et al., J. Immunol. Methods, 182: 41-50 (1995); Ames et al., J. Immunol. Methods, 184:177-186 (1995); Kettleborough et al., Eur. J. Immunol., 24: 952-958 (1994); Persic et al., Gene, 187: 9-18 (1997); Burton et al., Advances in Immunology, 57: 191-280 (1994); PCT Publication No. WO 92/01047; PCT Publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′, and F(ab′)₂ fragments can also be employed using methods known in the art such as those disclosed in PCT publication No. WO 92/22324; Mullinax et al., BioTechniques, 12(6): 864-869 (1992); Sawai et al., Am. J. Reprod. Immunol., 34: 26-34 (1995); and Better et al., Science, 240: 1041-1043 (1988). Examples of techniques which can be used to produce single-chain Fvs and antibodies include those described in U.S. Pat. Nos. 4,946,778 and 5,258,498; Huston et al., Methods in Enzymology, 203: 46-88 (1991); Shu et al., Proc. Natl. Acad. Sci. USA, 90: 7995-7999 (1993); and Skerra et al., Science, 240: 1038-1041 (1988).

Alternative to screening of recombinant antibody libraries by phage display, other methodologies known in the art for screening large combinatorial libraries can be applied to the identification of antibodies. One type of alternative expression system is one in which the recombinant antibody library is expressed as RNA-protein fusions, as described in PCT Publication No. WO 98/31700 (Szostak and Roberts), and in Roberts and Szostak, Proc. Natl. Acad. Sci. USA, 94: 12297-12302 (1997). In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above. A preferred example of this methodology is PROfusion display technology.

In another approach, the antibodies can also be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine). Examples of yeast display methods that can be used to make the antibodies include those disclosed in U.S. Pat. No. 6,699,658 (Wittrup et al.) incorporated herein by reference.

Production of Recombinant Antibodies

Antibodies may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies include Chinese Hamster Ovary (CHO cells) (including dhfr-CHO cells, described in Urlaub and Chasin, Proc. Natl. Acad. Sci. USA, 77: 4216-4220 (1980), used with a DHFR selectable marker, e.g., as described in Kaufman and Sharp, J. Mol. Biol., 159: 601-621 (1982), NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. In some aspects, the antibodies can be purified in CHO and/or HEK cells using routine techniques known in the art.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies. In addition, bifunctional antibodies may be produced in which one heavy and one light chain are an antibody (i.e., binds to an IgG antibody, IgM antibody and/or IgG or IgM antibody) and the other heavy and light chain are specific for an antigen other than an IgG antibody, IgM antibody and/or an IgG and IgM antibody by crosslinking an antibody to a second antibody by standard chemical crosslinking methods.

In a preferred system for recombinant expression of an antibody, or antigen-binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the disclosure provides a method of synthesizing a recombinant antibody by culturing a host cell in a suitable culture medium until a recombinant antibody is synthesized. The method can further comprise isolating the recombinant antibody from the culture medium.

Humanized Antibody

The humanized antibody may be an antibody or a variant, derivative, analog or portion thereof which immunospecifically binds to an antigen of interest and which comprises a framework (FR) region having substantially the amino acid sequence of a human antibody and a complementary determining region (CDR) having substantially the amino acid sequence of a non-human antibody. The humanized antibody may be from a non-human species antibody that binds the desired antigen having one or more complementarity determining regions (CDRs) from the non-human species and framework regions from a human immunoglobulin molecule.

As used herein, the term “substantially” in the context of a CDR refers to a CDR having an amino acid sequence at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. According to one aspect, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or of a heavy chain.

The humanized antibody can be selected from any class of immunoglobulins, including IgM, IgG, IgD, IgA and IgE, and any isotype, including without limitation IgG1, IgG2, IgG3, and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In one embodiment, such mutations, however, will not be extensive. Usually, at least 90%, at least 95%, at least 98%, or at least 99% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

The humanized antibody may be designed to minimize unwanted immunological response toward rodent anti-human antibodies, which limits the duration and effectiveness of therapeutic applications of those moieties in human recipients. The humanized antibody may have one or more amino acid residues introduced into it from a source that is non-human. These non-human residues are often referred to as “import” residues, which are typically taken from a variable domain. Humanization may be performed by substituting hypervariable region sequences for the corresponding sequences of a human antibody. Accordingly, such “humanized” antibodies are chimeric antibodies wherein substantially less than an intact human variable domain has been substituted by the corresponding sequence from a non-human species. For example, see U.S. Pat. No. 4,816,567, the contents of which are herein incorporated by reference. The humanized antibody may be a human antibody in which some hypervariable region residues, and possibly some FR residues are substituted by residues from analogous sites in rodent antibodies. Humanization or engineering of antibodies of the present disclosure can be performed using any known method, such as but not limited to those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

The humanized antibody may retain high affinity for a β-coronavirus (such as SARS-CoV and SARS-CoV-2) and other favorable biological properties. The humanized antibody may be prepared by a process of analysis of the parental sequences and various conceptual humanized products using three-dimensional models of the parental and humanized sequences. Three-dimensional immunoglobulin models are commonly available. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristics, such as increased affinity for β-coronavirus (such as SARS-CoV and SARS-CoV-2), is achieved. In general, the hypervariable region residues may be directly and most substantially involved in influencing antigen binding. As an alternative to humanization, human antibodies (also referred to herein as “fully human antibodies”) can be generated. For example, it is possible to isolate human antibodies from libraries via PROfusion and/or yeast related technologies. It is also possible to produce transgenic animals (e.g., mice that are capable, upon immunization, of producing a full repertoire of human antibodies in the absence of endogenous immunoglobulin production. For example, the homozygous deletion of the antibody heavy-chain joining region (JO gene in chimeric and germ-line mutant mice results in complete inhibition of endogenous antibody production. Transfer of the human germ-line immunoglobulin gene array in such germ-line mutant mice will result in the production of human antibodies upon antigen challenge. The humanized or fully human antibodies may be prepared according to the methods described in U.S. Pat. Nos. 5,770,429; 5,833,985; 5,837,243; 5,922,845; 6,017,517; 6,096,311; 6,111,166; 6,270,765; 6,303,755; 6,365,116; 6,410,690; 6,682,928; and 6,984,720, the contents each of which are herein incorporated by reference.

b. Recombinant Antigens

Synthetic Production of Isolated Nucleocapsid from a β-Coronavirus

Once sequenced, polypeptides, such as a nucleocapsid protein or fragment or variant thereof of a β-coronavirus (such as SARS-CoV or SARS-CoV-2); and/or (b) any protein, fragment or variant thereof that binds to an anti-nucleocapsid antibody or antibody variant thereof from a β-coronavirus, can be synthesized using methods known in the art, such as, for example, exclusive solid phase synthesis, partial solid phase synthesis, fragment condensation, and classical solution synthesis. See, e.g., Merrifield, J. Am. Chem. Soc. 85: 2149 (1963). On a solid phase, the synthesis typically begins from the C-terminal end of the peptide using an alpha-amino protected resin. A suitable starting material can be prepared, for instance, by attaching the required alpha-amino acid to a chloromethylated resin, a hydroxymethyl resin, or a benzhydrylamine resin. One such chloromethylated resin is sold under the tradename BIO-BEADS SX-1 by Bio Rad Laboratories (Richmond, Calif.), and the preparation of the hydroxymethyl resin is described by Bodonszky et al., Chem. Ind. (London) 38: 1597 (1966). The benzhydrylamine (BHA) resin has been described by Pietta and Marshall, Chem. Comm 650 (1970) and is commercially available from Beckman Instruments, Inc. (Palo Alto, Calif.) in the hydrochloride form. Automated peptide synthesizers are commercially available, as are services that make peptides to order.

Thus, the polypeptides can be prepared by coupling an alpha-amino protected amino acid to the chloromethylated resin with the aid of, for example, cesium bicarbonate catalyst, according to the method described by Gisin, Hely. Chim. Acta. 56: 1467 (1973). After the initial coupling, the alpha-amino protecting group is removed by a choice of reagents including trifluoroacetic acid (TFA) or hydrochloric acid (HC1) solutions in organic solvents at room temperature.

Suitable alpha-amino protecting groups include those known to be useful in the art of stepwise synthesis of peptides. Examples of alpha-amino protecting groups are: acyl type protecting groups (e.g., formyl, trifluoroacetyl, and acetyl), aromatic urethane type protecting groups (e.g., benzyloxycarbonyl (Cbz) and substituted Cbz), aliphatic urethane protecting groups (e.g., t-butyloxycarbonyl (Boc), isopropyloxycarbonyl, and cyclohexyloxycarbonyl), and alkyl type protecting groups (e.g., benzyl and triphenylmethyl). Boc and Fmoc are preferred protecting groups. The side chain protecting group remains intact during coupling and is not split off during the deprotection of the amino-terminus protecting group or during coupling. The side chain protecting group must be removable upon the completion of the synthesis of the final peptide and under reaction conditions that will not alter the target peptide.

After removal of the alpha-amino protecting group, the remaining protected amino acids are coupled stepwise in the desired order. An excess of each protected amino acid is generally used with an appropriate carboxyl group activator such as dicyclohexylcarbodiimide (DCC) in solution, for example, in methylene chloride and dimethyl formamide (DMF) mixtures.

After the desired amino acid sequence has been completed, the desired peptide is decoupled from the resin support by treatment with a reagent, such as TFA or hydrogen fluoride (HF), which not only cleaves the peptide from the resin, but also cleaves all remaining side chain protecting groups. When the chloromethylated resin is used, HF treatment results in the formation of the free peptide acids. When the benzhydrylamine resin is used, HF treatment results directly in the free peptide amide. Alternatively, when the chloromethylated resin is employed, the side chain protected peptide can be decoupled by treatment of the peptide resin with ammonia to give the desired side chain protected amide or with an alkylamine to give a side chain protected alkylamide or dialkylamide. Side chain protection is then removed in the usual fashion by treatment with hydrogen fluoride to give the free amides, alkylamides, or dialkylamides.

These and other solid phase peptide synthesis procedures are well-known in the art. Such procedures are also described by Stewart and Young in Solid Phase Peptide Syntheses (2nd Ed., Pierce Chemical Company, 1984).

Recombinant Production of Isolated Nucleocapsid from a β-Coronavirus

All or a portion of (a) a nucleocapsid protein or a fragment or variant thereof from a β-coronavirus; and/or (b) any protein, fragment or variant thereof that binds to an anti-nucleocapsid antibody or antibody variant thereof from a β-coronavirus, can be isolated or purified, or recombinantly produced, using methods known in the art. Such protein or fragments may be employed, e.g., as calibrators or controls, or as quality control reagents. For example, an isolated or purified nucleic acid molecule comprising a nucleotide sequence encoding the polypeptide can be expressed in a host cell, and the polypeptide can be isolated. The isolated or purified nucleic acid molecule can comprise a nucleotide sequence encoding (a) a nucleocapsid protein or a fragment or variant thereof from a β-coronavirus; and/or (b) any protein, fragment or variant thereof that binds to an anti-nucleocapsid antibody or antibody variant thereof from a β-coronavirus. In one aspect, the isolated or purified nucleic acid can comprise a nucleotide sequence encoding a nucleocapsid protein having the amino acid sequence of SEQ ID NO:1 or a fragment or variant thereof. The isolated or purified nucleic acid molecule can be a vector.

The isolated nucleic acid can be synthesized with an oligonucleotide synthesizer, for example. One of ordinary skill in the art will readily appreciate that, due to the degeneracy of the genetic code, more than one nucleotide sequence can encode a given amino acid sequence. In this regard, a nucleotide sequence encoding an amino acid sequence that is substantially identical to an amino acid sequence of a SEQ ID NO. specified herein can be used. Codons, which are favored by a given host cell, preferably are selected for recombinant production. A nucleotide sequence encoding the amino acid sequence of a specified SEQ ID NO. can be combined with other nucleotide sequences using polymerase chain reaction (PCR), ligation, or ligation chain reaction (LCR) to encode a mutated truncated nucleocapsid and/or spike polypeptide. The individual oligonucleotides typically contain 5′ or 3′ overhangs for complementary assembly. Once assembled, the nucleotide sequence encoding (a) a nucleocapsid protein or a fragment or variant thereof from a β-coronavirus; and/or (b) any protein, fragment or variant thereof that binds to an anti-nucleocapsid antibody or antibody variant thereof from a β-coronavirus can be inserted into a vector, operably linked to control sequences as necessary for expression in a given host cell, and introduced (such as by transformation or transfection) into a host cell. The nucleotide sequence can be further manipulated (for example, linked to one or more nucleotide sequences encoding additional immunoglobulin domains, such as additional constant regions) and/or expressed in a host cell.

Although not all vectors and expression control sequences may function equally well to express a polynucleotide sequence of interest and not all hosts function equally well with the same expression system, it is believed that those skilled in the art will be able to make a selection among these vectors, expression control sequences, optimized codons, and hosts for use in the present disclosure without any undue experimentation. For example, in selecting a vector, the host must be considered because the vector must be able to replicate in it or be able to integrate into the chromosome. The vector's copy number, the ability to control that copy number, and the expression of any other proteins encoded by the vector, such as antibiotic markers, should also be considered. In selecting an expression control sequence, a variety of factors also can be considered. These include, but are not limited to, the relative strength of the sequence, its controllability, and its compatibility with the nucleotide sequence encoding (a) a nucleocapsid protein or a fragment or variant thereof from a β-coronavirus; and/or (b) any protein, fragment or variant thereof that binds to an anti-nucleocapsid antibody or antibody variant thereof from a 62 -coronavirus, particularly with regard to potential secondary structures. Hosts should be selected by consideration of their compatibility with the chosen vector, their codon usage, their secretion characteristics, their ability to fold the polypeptide correctly, their fermentation or culture requirements, their ability (or lack thereof) to glycosylate the protein, and the ease of purification of the products encoded by the nucleotide sequence, etc.

The recombinant vector can be an autonomously replicating vector, namely, a vector existing as an extrachromosomal entity, the replication of which is independent of chromosomal replication (such as a plasmid). Alternatively, the vector can be one which, when introduced into a host cell, is integrated into the host cell genome and replicated together with the chromosome(s) into which it has been integrated.

The vector is preferably an expression vector in which the polynucleotide sequence encoding (a) a nucleocapsid protein or a fragment or variant thereof from a β-coronavirus; and/or (b) any protein, fragment or variant thereof that binds to an anti-nucleocapsid antibody or antibody variant thereof from a β-coronavirus, is operably linked to additional segments required for transcription of the polynucleotide sequence. The vector is typically derived from plasmid or viral DNA. A number of suitable expression vectors for expression in the host cells mentioned herein are commercially available or described in the literature. Useful expression vectors for eukaryotic hosts, include, but are not limited to, vectors comprising expression control sequences from SV40, bovine papilloma virus, adenovirus and cytomegalovirus. Specific vectors include pcDNA3.1 (+)\Hyg (Invitrogen Corp., Carlsbad, Calif.) and pCI-neo (Stratagene, La Jolla, Calif.). Examples of expression vectors for use in yeast cells include, but are not limited to, the plasmid and derivatives thereof, the POT1 vector (see, e.g., U.S. Pat. No. 4,931,373), the pJSO37 vector (described in Okkels, Ann New York Acad. Sci. 782: 202-207 (1996)) and pPICZ A, B or C (Invitrogen). Examples of expression vectors for use in insect cells include, but are not limited to, pVL941, pBG311 (Cate et al., Cell 45: 685-698 (1986)), and pBluebac 4.5 and pMelbac (both of which are available from Invitrogen).

Other vectors that can be used allow the nucleotide sequence encoding (a) a nucleocapsid protein or a fragment or variant thereof from a β-coronavirus; and/or (b) any protein, fragment or variant thereof that binds to an anti-nucleocapsid antibody or antibody variant thereof from a β-coronavirus, to be amplified in copy number. Such amplifiable vectors are well-known in the art. These vectors include, but are not limited to, those vectors that can be amplified by dihydrofolate reductase (DHFR) amplification (see, for example, Kaufman, U.S. Pat. No. 4,470,461; and Kaufman et al., Mol. Cell. Biol. 2: 1304-1319 (1982)) and glutamine synthetase (GS) amplification (see, for example, U.S. Pat. No. 5,122,464 and EP Patent Application Publication No. 0 338 841).

The recombinant vector can further comprise a nucleotide sequence enabling the vector to replicate in the host cell in question. An example of such a sequence for use in a mammalian host cell is the SV40 origin of replication. Suitable sequences enabling the vector to replicate in a yeast cell are the yeast plasmid 2μ replication genes REP 1-3 and origin of replication.

The vector can also comprise a selectable marker, namely, a gene or polynucleotide, the product of which complements a defect in the host cell, such as the gene coding for DHFR or the Schizosaccharomyces pombe TPI gene (see, e.g., Russell, Gene 40: 125-130 (1985)), or one which confers resistance to a drug, such as ampicillin, kanamycin, tetracycline, chloramphenicol, neomycin, hygromycin or methotrexate. For filamentous fungi, selectable markers include, but are not limited to, amdS, pyrG, arcB, niaD and sC.

Also present in the vector are “control sequences,” which are any components that are necessary or advantageous for the expression of (a) the nucleocapsid protein or a fragment or variant thereof from a β-coronavirus; and/or (b) any protein, fragment or variant thereof that binds to an anti-nucleocapsid antibody or antibody variant thereof from a β-coronavirus. Each control sequence can be native or foreign to the nucleotide sequence encoding (a) the nucleocapsid protein or a fragment or variant thereof from a β-coronavirus; and/or (b) any protein, fragment or variant thereof that binds to an anti-nucleocapsid antibody or antibody variant thereof from a β-coronavirus. Such control sequences include, but are not limited to, a leader, a polyadenylation sequence, a propeptide sequence, a promoter, an enhancer or an upstream activating sequence, a signal peptide sequence, and a transcription terminator. At a minimum, the control sequences include at least one promoter operably linked to the polynucleotide sequence encoding (a) the nucleocapsid protein or a fragment or variant thereof from a β-coronavirus; and/or (b) any protein, fragment or variant thereof that binds to an anti-nucleocapsid antibody or antibody variant thereof from a β-coronavirus.

By “operably linked” is meant the covalent joining of two or more nucleotide sequences, by means of enzymatic ligation or otherwise, in a configuration relative to one another such that the normal function of the sequences can be performed. For example, a nucleotide sequence encoding a presequence or secretory leader is operably linked to a nucleotide sequence for a polypeptide if it is expressed as a preprotein that participates in the secretion of the polypeptide; a promoter or enhancer is operably linked to a coding sequence if it affects the transcription of the sequence; a ribosome binding site is operably linked to a coding sequence if it is positioned so as to facilitate translation. Generally, “operably linked” means that the nucleotide sequences being linked are contiguous and, in the case of a secretory leader, contiguous and in the same reading frame. Linking is accomplished by ligation at convenient restriction sites. If such sites do not exist, then synthetic oligonucleotide adaptors or linkers can be used, in conjunction with standard recombinant DNA methods.

A wide variety of expression control sequences can be used in the context of the present disclosure. Such useful expression control sequences include the expression control sequences associated with structural genes of the foregoing expression vectors as well as any sequence known to control the expression of genes of prokaryotic or eukaryotic cells or their viruses, and various combinations thereof. Examples of suitable control sequences for directing transcription in mammalian cells include the early and late promoters of SV40 and adenovirus, for example, the adenovirus 2 major late promoter, the MT-1 (metallothionein gene) promoter, the human cytomegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus E1b region polyadenylation signals and the Kozak consensus sequence (Kozak, J. Mol. Biol. 196: 947-50 (1987)).

In order to improve expression in mammalian cells a synthetic intron can be inserted in the 5′ untranslated region of a polynucleotide sequence encoding the antibody or a fragment thereof. An example of a synthetic intron is the synthetic intron from the plasmid pCI-Neo (available from Promega Corporation, Madison, Wis.).

Examples of suitable control sequences for directing transcription in insect cells include, but are not limited to, the polyhedrin promoter, the P10 promoter, the baculovirus immediate early gene 1 promoter, the baculovirus 39K delayed-early gene promoter, and the SV40 polyadenylation sequence.

Examples of suitable control sequences for use in yeast host cells include the promoters of the yeast α-mating system, the yeast triose phosphate isomerase (TPI) promoter, promoters from yeast glycolytic genes or alcohol dehydrogenase genes, the ADH2-4-c promoter and the inducible GAL promoter. 102111 Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulas glucoamylase, A. nidulans acetamidase, Rhizomucor miehei aspartic proteinase or lipase, the TPI1 terminator, and the ADH3 terminator.

The polynucleotide sequence may or may not also include a polynucleotide sequence that encodes a signal peptide. The signal peptide is present when (a) the nucleocapsid protein or a fragment or variant thereof from a β-coronavirus; and/or (b) any protein, fragment or variant thereof that binds to an anti-nucleocapsid antibody or antibody variant thereof from a β-coronavirus. Such signal peptide, if present, should be one recognized by the cell chosen for expression of the polypeptide. The signal peptide can be homologous or heterologous to (a) the nucleocapsid protein or a fragment or variant thereof from a β-coronavirus; and/or (b) any protein, fragment or variant thereof that binds to an anti-nucleocapsid antibody or antibody variant thereof from a β-coronavirus or can be homologous or heterologous to the host cell, i.e., a signal peptide normally expressed from the host cell or one which is not normally expressed from the host cell. Accordingly, the signal peptide can be prokaryotic, for example, derived from a bacterium, or eukaryotic, for example, derived from a mammalian, insect, filamentous fungal, or yeast cell.

The presence or absence of a signal peptide will, for example, depend on the expression host cell used for the production of (a) the nucleocapsid protein or a fragment or variant thereof from a β-coronavirus; and/or (b) any protein, fragment or variant thereof that binds to an anti-nucleocapsid antibody or antibody variant thereof from a β-coronavirus. For use in filamentous fungi, the signal peptide can conveniently be derived from a gene encoding an Aspergillus sp. amylase or glucoamylase, a gene encoding a Rhizomucor miehei lipase or protease or a Humicola lanuginosa lipase. For use in insect cells, the signal peptide can be derived from an insect gene (see, e.g., WO 90/05783), such as the lepidopteran Manduca sexta adipokinetic hormone precursor (see, e.g., U.S. Pat. No. 5,023,328), the honeybee melittin (Invitrogen), ecdysteroid UDP glucosyltransferase (egt) (Murphy et al., Protein Expression and Purification 4: 349-357 (1993), or human pancreatic lipase (hpl) (Methods in Enzymology 284: 262-272 (1997)).

Specific examples of signal peptides for use in mammalian cells include murine Ig kappa light chain signal peptide (Coloma, J. Imm Methods 152: 89-104 (1992)). Suitable signal peptides for use in yeast cells include the a-factor signal peptide from S. cerevisiae (see, e.g., U.S. Pat. No. 4,870,008), the signal peptide of mouse salivary amylase (see, e.g., Hagenbuchle et al., Nature 289: 643-646 (1981)), a modified carboxypeptidase signal peptide (see, e.g., Valls et al., Cell 48: 887-897 (1987)), the yeast BAR1 signal peptide (see, e.g., WO 87/02670), and the yeast aspartic protease 3 (YAPS) signal peptide (see, e.g., Egel-Mitani et al., Yeast 6: 127-137 (1990)).

In view of the above, the above-described isolated or purified nucleic acid molecule, which can be a vector, can be introduced into a host cell as described herein below. Accordingly, a host cell comprising the isolated or purified nucleic acid molecule is provided.

Any suitable host can be used to produce (a) the nucleocapsid protein or a fragment or variant thereof from a β-coronavirus; and/or (b) any protein, fragment or variant thereof that binds to an anti-nucleocapsid antibody or antibody variant thereof from a β-coronavirus, including bacteria, fungi (including yeasts), plant, insect, mammal or other appropriate animal cells or cell lines, as well as transgenic animals or plants. A preferred host cell is a Chinese hamster ovary (CHO) cell. Examples of bacterial host cells include, but are not limited to, gram-positive bacteria, such as strains of Bacillus, for example, B. brevis or B. subtilis, Pseudomonas or Streptomyces, or gram-negative bacteria, such as strains of E. coli. The introduction of a vector into a bacterial host cell can, for instance, be effected by protoplast transformation (see, for example, Chang et al., Molec. Gen. Genet. 168: 111-115 (1979)), using competent cells (see, for example, Young et al., J. of Bacteriology 81: 823-829 (1961), or Dubnau et al., J. of Molec. Biol. 56: 209-221 (1971)), electroporation (see, for example, Shigekawa et al., Biotechniques 6: 742-751 (1988)), or conjugation (see, for example, Koehler et al., J. of Bacteriology 169: 5771-5278 (1987)).

Examples of suitable filamentous fungal host cells include, but are not limited to, strains of Aspergillus, for example, A. oryzae, A. niger, or A. nidulans, Fusarium or Trichoderma. Fungal cells can be transformed by a process involving protoplast formation, transformation of the protoplasts, and regeneration of the cell wall using techniques known to those ordinarily skilled in the art. Suitable procedures for transformation of Aspergillus host cells are described in EP Patent Application No. 0 238 023 and U.S. Pat. No. 5,679,543. Suitable methods for transforming Fusarium species are described by Malardier et al., Gene 78: 147-156 (1989), and WO 96/00787. Yeast can be transformed using the procedures described by Becker and Guarente, In Abelson, J. N. and Simon, M. I., editors, Guide to Yeast Genetics and Molecular Biology, Methods in Enzymology 194: 182-187, Academic Press, Inc., New York; Ito et al, J. of Bacteriology 153: 163 (1983); and Hinnen et al., PNAS USA 75: 1920 (1978).

Examples of suitable yeast host cells include strains of Saccharomyces, for example, S. cerevisiae, Schizosaccharomyces, Klyveromyces, Pichia, such as P. pastoris or P. methanolica, Hansenula, such as H. polymorpha or yarrowia. Methods for transforming yeast cells with heterologous polynucleotides and producing heterologous polypeptides therefrom are disclosed by Clontech Laboratories, Inc, Palo Alto, Calif., USA (in the product protocol for the Yeastmaker™ Yeast Tranformation System Kit), and by Reeves et al., FEMS Microbiology Letters 99: 193-198 (1992), Manivasakam et al., Nucleic Acids Research 21: 4414-4415 (1993), and Ganeva et al., FEMS Microbiology Letters 121: 159-164 (1994).

Examples of suitable insect host cells include, but are not limited to, a Lepidoptora cell line, such as Spodoptera frupperda (Sf9 or Sf21) or Trichoplusia ni cells (High Five) (see, e.g., U.S. Pat. No. 5,077,214). Transformation of insect cells and production of heterologous polypeptides are well-known to those skilled in the art.

Examples of suitable mammalian host cells include Chinese hamster ovary (CHO) cell lines, simian (e.g., Green Monkey) cell lines (COS), mouse cells (for example, NS/O), baby hamster kidney (BHK) cell lines, human cells (such as human embryonic kidney (HEK) cells (e.g., HEK 293 cells (A.T.C.C. Accession No. CRL-1573)), myeloma cells that do not otherwise produce immunoglobulin protein, and plant cells in tissue culture. Preferably, the mammalian host cells are CHO cell lines and/or HEK (e.g., HEK 293) cell lines. Another preferred host cell is the B3.2 cell line (e.g., Abbott Laboratories, Abbott Bioresearch Center, Worcester, Mass.), or another dihydrofolate reductase deficient (DHFR⁻) CHO cell line (e.g., available from Invitrogen). 102271 Methods for introducing exogenous polynucleotides into mammalian host cells include calcium phosphate-mediated transfection, electroporation, DEAE-dextran mediated transfection, liposome-mediated transfection, viral vectors and the transfection method described by Life Technologies Ltd, Paisley, UK using Lipofectamine™ 2000. These methods are well-known in the art and are described, for example, by Ausbel et al. (eds.), Current Protocols in Molecular Biology, John Wiley & Sons, New York, USA (1996). The cultivation of mammalian cells is conducted according to established methods, e.g., as disclosed in Jenkins, Ed., Animal Cell Biotechnology, Methods and Protocols, Human Press Inc. Totowa, N.J., USA (1999), and Harrison and Rae, General Techniques of Cell Culture, Cambridge University Press (1997).

In the production methods, cells are cultivated in a nutrient medium suitable for production of the (a) nucleocapsid protein or a fragment or variant thereof from a β-coronavirus; and/or (b) any protein, fragment or variant thereof that binds to an anti-nucleocapsid antibody or antibody variant thereof from a β-coronavirus using methods known in the art. For example, cells are cultivated by shake flask cultivation, small-scale or large-scale fermentation (including continuous, batch, fed-batch, or solid state fermentations) in laboratory or industrial fermenters performed in a suitable medium and under conditions allowing (a) the nucleocapsid protein or a fragment or variant thereof from a β-coronavirus; and/or (b) any protein, fragment or variant thereof that binds to an anti-nucleocapsid antibody or antibody variant thereof from a β-coronavirus to be expressed and/or isolated. The cultivation takes place in a suitable nutrient medium comprising carbon and nitrogen sources and inorganic salts, using procedures known in the art. Suitable media are available from commercial suppliers or can be prepared according to published compositions (e.g., in catalogues of the American Type Culture Collection). If (a) the nucleocapsid protein or a fragment or variant thereof from a β-coronavirus; and/or (b) any protein, fragment or variant thereof that binds to an anti-nucleocapsid antibody or antibody variant thereof from a β-coronavirus is secreted into the nutrient medium, it can be recovered directly from the medium.

The resulting (a) nucleocapsid protein or a fragment or variant thereof from a β-coronavirus; and/or any (b) protein, fragment or variant thereof that binds to an anti-nucleocapsid antibody or antibody variant thereof from a β-coronavirus can be recovered by methods known in the art. For example, (a) the nucleocapsid protein or a fragment or variant thereof from a β-coronavirus; and/or (b) any protein, fragment or variant thereof that binds to an anti-nucleocapsid antibody or antibody variant thereof from a β-coronavirus can be recovered from the nutrient medium by conventional procedures including, but not limited to, centrifugation, filtration, extraction, spray drying, evaporation, or precipitation. The (a) nucleocapsid protein or a fragment or variant thereof from a β-coronavirus; and/or (b) any protein, fragment or variant thereof that binds to an anti-nucleocapsid antibody or antibody variant thereof from a β-coronavirus can be purified by a variety of procedures known in the art including, but not limited to, chromatography (such as, but not limited to, ion exchange, affinity, hydrophobic, chromatofocusing, and size exclusion), electrophoretic procedures (such as, but not limited to, preparative isoelectric focusing), differential solubility (such as, but not limited to, ammonium sulfate precipitation), SDS-PAGE, or extraction (see, for example, Janson and Ryden, editors, Protein Purification, VCH Publishers, New York (1989)). In some aspects, the purification can be done in CHO and/or HEK cells using routine techniques known in the art. Examples of antibodies that bind to the nucleocapsid protein or fragment or variant of SARS-CoV-2 and can be used in the methods described herein (either as capture and/or detection agents) include (i) CR 3001, CR3002, CR3006, CR3013, CR3014 and CR3018 described in van den Brink et al., Journal of Vir. 79(3):1635-1644 (Feb. 2005), the contents of which are herein incorporated by reference as well as the antibodies described in U.S. Pat. No. 7,696,330; and/or (ii) scFv antibody N18 (described in Zhao, et al., Microbes and Infection, 9:1026-1033 (2007), the contents of each of which are also herein incorporated by reference.

### 5. VARIATIONS ON METHODS

The disclosed methods detect the presence or determine the amount or level of at least one SARS-CoV-2 nucleocapsid protein present in a biological sample as described herein. The methods may also be adapted in view of other methods for analyzing analytes. Examples of well-known variations include, but are not limited to, immunoassay, competitive inhibition immunoassay (e.g., forward and reverse), enzyme multiplied immunoassay technique (EMIT), a competitive binding assay, bioluminescence resonance energy transfer (BRET), one-step antibody detection assay, homogeneous assay, heterogeneous assay, capture on the fly assay, single molecule detection assay, lateral flow assay, etc.

a. Immunoassay

The analyte of interest, namely, at least one SARS-CoV-2 nucleocapsid protein, may be analyzed using at least one first specific binding partner (e.g., at least one anti-SARS-CoV antibody or anti-SARS-CoV-2 antibody) and at least one second specific binding partner (e.g., at least one anti-SARS-CoV antibody or anti-SARS-CoV-2 antibody) in an immunoassay. The presence or amount of the analyte, namely, at least one SARS-CoV-2 nucleocapsid protein, can be determined using the binding of the at least one first specific binding partner (e.g., at least one anti-SARS-CoV antibody or anti-SARS-CoV-2 antibody) and at least one second specific binding partner to the analyte (e.g., at least one anti-SARS-CoV antibodyor anti-SARS-CoV-2 antibody). For example, at least anti-SARS-CoV antibody or anti-SARS-CoV-2 antibody (e.g., at least one first specific binding partner) may specifically bind to the analyte (e.g., at least one SARS-CoV-2 nucleocapsid protein)). One or more anti-SARS-CoV or anti-SARS-CoV-2 antibodies labeled with at least one detectable label (e.g., second specific binding partner) can used to detect the presence of determine the amount of the analyte in the biological sample.

The presence or amount of the analyte (e.g., a SARS-CoV-2 nucleocapsid protein present in a biological sample may be readily determined using an immunoassay. For example, in one aspect, one method that can be used is a chemiluminescent microparticle immunoassay, in particular one employing the ARCHITECT® or Alinity automated analyzer (Abbott Laboratories, Abbott Park, Ill.), as an example. Other methods that can be used include, for example, mass spectrometry, and immunohistochemistry (e.g., with sections from tissue biopsies). Additionally, methods of detection include those described in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Specific immunological binding of the antibody to an analyte (e.g., a SARS-CoV-2 nucleocapsid protein) can be detected via direct labels, such as fluorescent or luminescent tags, metals and radionuclides attached to the antibody or via indirect labels, such as alkaline phosphatase or horseradish peroxidase.

The use of immobilized antibodies (e.g., at least one first specific and/or second specific binding partner) or antibody fragments thereof (e.g., at least one second specific binding partner) may be incorporated into the immunoassay. The antibodies may be immobilized onto a variety of supports, such as magnetic or chromatographic matrix particles, the surface of an assay plate (such as microtiter wells), pieces of a solid substrate material, and the like. An assay strip can be prepared by coating the antigen and/or antibody or plurality of antibodies in an array on a solid support. This strip can then be dipped into the test sample and processed quickly through washes and detection steps to generate a measurable signal, such as a colored spot.

A homogeneous format may be used. For example, after the biological sample is obtained from a subject, a mixture is prepared. The mixture contains the test sample being assessed for the analyte (e.g., a SARS-CoV-2 nucleocapsid protein), a first specific binding partner, and a second specific binding partner. The order in which the test sample, the first specific binding partner, and the second specific binding partner are added to form the mixture is not critical. The test sample is simultaneously contacted with the first specific binding partner and the second specific binding partner. In some embodiments, the first specific binding partner and any SARS-CoV-2 nucleocapsid protein contained in the test sample may form a first specific binding partner-analyte (e.g., SARS-CoV-2 nucleocapsid protein)-complex and the second specific binding partner may form a first specific binding partner-analyte of interest (e.g., SARS-CoV-2 nucleocapsid protein)-second specific binding partner complex. Moreover, the second specific binding partner is labeled with or contains a detectable label as described above.

A heterogeneous format may be used. For example, after the biological sample is obtained from a subject, a first mixture is prepared. The mixture contains the biological sample being assessed for the analyte (e.g., SARS-CoV-2 nucleocapsid protein) and a first specific binding partner, wherein the first specific binding partner and any SARS-CoV-2 nucleocapsid protein in the biological sample form a first specific binding partner-analyte (SARS-CoV-2 nucleocapsid protein)-complex. The order in which the biological sample and the first specific binding partner are added to form the mixture is not critical.

The first specific binding partner (e.g., at least one anti-SARS-CoV antibody, at least one anti-SARS-CoV-2 antibody, or fragment thereof) may be immobilized on a solid phase. The solid phase used in the immunoassay (for the first specific binding partner and, optionally, the second specific binding partner) can be any solid phase known in the art, such as, but not limited to, a magnetic particle, a bead, a test tube, a microtiter plate, a cuvette, a membrane, a scaffolding molecule, a film, a filter paper, a disc, and a chip. In those embodiments where the solid phase is a bead, the bead may be a magnetic bead or a magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fe. Examples of ferrimagnetic materials include NiFe₂O₄, CoFe₂O₄, Fe₃O₄ (or FeO.Fe₂O₃). Beads can have a solid core portion that is magnetic and is surrounded by one or more non-magnetic layers. Alternately, the magnetic portion can be a layer around a non-magnetic core. The solid support on which the first specific binding partner is immobilized may be stored in dry form or in a liquid. The magnetic beads may be subjected to a magnetic field prior to or after contacting with the sample with a magnetic bead on which the first specific binding partner is immobilized.

After the mixture containing the first specific binding partner-analyte (e.g, at least one SARS-CoV-2 nucleocapsid protein) complex is formed, any unbound analyte (e.g., SARS-CoV-2 nucleocapsid protein) is removed from the complex using any technique known in the art. For example, the unbound analyte (e.g., SARS-CoV-2 nucleocapsid protein) can be removed by washing. Desirably, in some instances, the first specific binding partner is present in excess of any analyte (e.g., SARS-CoV-2 nucleocapsid protein) present in the test sample, such that all or most analyte (e.g., SARS-CoV-2 nucleocapsid protein) that is present in the test sample is bound by the first specific binding partner.

After any unbound analyte (e.g., SARS-CoV-2 nucleocapsid protein) is removed, a second specific binding partner is added to the mixture to form a first specific binding partner-analyte of interest (e.g., SARS-CoV-2 nucleocapsid protein)-second specific binding partner complex. Moreover, the second specific binding partner is labeled with or contains a detectable label as described above.

The use of immobilized antibodiesor antibody fragments thereof may be incorporated into the immunoassay. The antibodies may be immobilized onto a variety of supports, such as magnetic or chromatographic matrix particles (such as a magnetic bead), latex particles or modified surface latex particles, polymer or polymer film, plastic or plastic film, planar substrate, the surface of an assay plate (such as microtiter wells), pieces of a solid substrate material, and the like. The antibody (antibodies)or fragments thereof can be bound to the solid support by adsorption, by covalent bonding using a chemical coupling agent or by other means known in the art, provided that such binding does not interfere with the ability of the antibody to bind analyte (e.g., SARS-CoV-2 nucleocapsid protein). An assay strip can be prepared by coating the antibody or plurality of antibodies in an array on a solid support. This strip can then be dipped into the test sample and processed quickly through washes and detection steps to generate a measurable signal, such as a colored spot.

b. Forward Competitive Inhibition Assay

In a forward competitive format, an aliquot of labeled analyte of interest (e.g., SARS-CoV-2 nucleocapsid protein) having a fluorescent label, a tag attached with a cleavable linker, etc.) of a known concentration is used to compete with analyte of interest (e.g., SARS-CoV-2 nucleocapsid protein) in a biological sample for binding to antibody directed against analyte of interest.

In a forward competition assay, an immobilized specific binding partner (such as an antibody) can either be sequentially or simultaneously contacted with the biological sample and a labeled analyte of interest, labeled analyte of interest fragment or labeled analyte of interest variant thereof. The analyte of interest, analyte of interest fragment or analyte of interest variant, can be labeled with any detectable label, including a detectable label comprised of tag attached with a cleavable linker. In this assay, the antibody can be immobilized on to a solid support. Alternatively, the antibody can be coupled to an antibody, such as an antispecies antibody, that has been immobilized on a solid support, such as a microparticle or planar substrate.

The labeled analyte of interest, the biological sample and the antibody are incubated at a pH of from about 4.5 to about 10.0, at a temperature of from about 2° C. to about 45° C., and for a period from at least one (1) minute to about eighteen (18) hours, from about 2-6 minutes, from about 7-12 minutes, from a bout 5-15 minutes, or from about 3-4 minutes. Two different species of antibody and/or recombinant antigen-analyte of interest complexes may then be generated. Specifically, one of the antibody and/or recombinant antigen-analyte of interest complexes generated contains a detectable label (e.g., a fluorescent label, etc.) while the other antibody and/or recombinant antigen-analyte of interest complex does not contain a detectable label. The antibody and/or recombinant antigen-analyte of interest complex can be, but does not have to be, separated from the remainder of the biological sample prior to quantification of the detectable label. Regardless of whether the antibody and/or recombinant antigen-analyte of interest complex is separated from the remainder of the biological sample, the amount of detectable label in the antibody and/or recombinant antigen-analyte of interest complex is then quantified. The concentration of analyte of interest in the biological sample can then be determined as described above.

c. Reverse Competitive Inhibition Assay

In a reverse competition assay, an immobilized analyte of interest (e.g. a SARS-CoV-2 nucleocapsid protein) can either be sequentially or simultaneously contacted with a test sample and at least one labeled antibody.

The analyte of interest can be bound to a solid support, such as the solid supports discussed above.

The immobilized analyte of interest, biological sample and at least one labeled antibody are incubated under conditions similar to those described above. Two different species of analyte of interest-antibody complexes are then generated. Specifically, one of the analyte of interest-antibody and/or recombinant antigen complexes generated is immobilized and contains a detectable label (e.g., a fluorescent label, etc.) while the other analyte of interest-antibody complex is not immobilized and contains a detectable label. The non-immobilized analyte of interest-antibody complex and the remainder of the biological sample are removed from the presence of the immobilized analyte of interest-antibody complex through techniques known in the art, such as washing. Once the non-immobilized analyte of interest antibody and/or recombinant antigen complex is removed, the amount of detectable label in the immobilized analyte of interest-antibody complex is then quantified following cleavage of the tag. The concentration of analyte of interest in the test sample can then be determined by comparing the quantity of detectable label as described above.

d. One-Step Immunoassay or “Capture on the Fly” Assay

In a capture on the fly immunoassay, a solid substrate is pre-coated with an immobilization agent. The capture agent, the analyte (e.g., SARS-CoV-2 nucleocapsid protein) and the detection agent are added to the solid substrate together, followed by a wash step prior to detection. The capture agent can bind the analyte (e.g., SARS-CoV-2 nucleocapsid protein) and comprises a ligand for an immobilization agent. The capture agent and the detection agents may be antibodies or any other moiety capable of capture or detection as described herein or known in the art. The ligand may comprise a peptide tag and an immobilization agent may comprise an anti-peptide tag antibody. Alternately, the ligand and the immobilization agent may be any pair of agents capable of binding together so as to be employed for a capture on the fly assay (e.g., specific binding pair, and others such as are known in the art). More than one analyte may be measured. In some embodiments, the solid substrate may be coated with an antibody and the analyte to be analyzed is an antigen.

In certain other embodiments, in a one-step immunoassay or “capture on the fly”, a solid support (such as a microparticle) pre-coated with an immobilization agent (such as biotin, streptavidin, etc.) and at least a first specific binding partner and a second specific binding partner (which function as capture and detection reagents, respectively) are used. The first specific binding partner comprises a ligand for the immobilization agent (for example, if the immobilization agent on the solid support is streptavidin, the ligand on the first specific binding partner may be biotin) and also binds to the analyte of interest e.g., SARS-CoV-2 nucleocapsid protein). The second specific binding partner comprises a detectable label and binds to an analyte of interest (e.g., SARS-CoV-2 nucleocapsid protein). The solid support and the first and second specific binding partners may be added to a test sample (either sequentially or simultaneously). The ligand on the first specific binding partner binds to the immobilization agent on the solid support to form a solid support/first specific binding partner complex. Any analyte of interest present in the sample binds to the solid support/first specific binding partner complex to form a solid support/first specific binding partner/analyte complex. The second specific binding partner binds to the solid support/first specific binding partner/analyte complex and the detectable label is detected. An optional wash step may be employed before the detection. In certain embodiments, in a one-step assay more than one analyte may be measured. In certain other embodiments, more than two specific binding partners can be employed. In certain other embodiments, multiple detectable labels can be added. In certain other embodiments, multiple analytes of interest can be detected, or their amounts, levels or concentrations, measured, determined or assessed.

The use of a capture on the fly assay can be done in a variety of formats as described herein, and known in the art. For example, the format can be a sandwich assay such as described above, but alternately can be a competition assay, can employ a single specific binding partner, or use other variations such as are known.

e. Single Molecule Detection Assay

Single molecule detection assays and methods, such as the use of a nanopore device or nanowell device, can also be used. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety. Other devices and methods appropriate for single molecule detection can also be employed.

f. Lateral Flow Assays

Lateral flow assays are generally provided in a device comprising a lateral flow test strip (e.g., nitrocellulose or filter paper), a sample application area (e.g., sample pad), a test results area (e.g., a test line), an optional control results area (e.g., a control line), and an analyte-specific binding partner that is bound to a detectable label (e.g., a colored particle or an enzyme detection system). See, e.g., U.S. Pat. Nos. 6,485,982; 6,187,598; 5,622,871; 6,565,808; and 6,809,687; and U.S. patent application Ser. No. 10/717,082, each of which is incorporated herein by reference.

In some aspects, the present disclosure provides assays for detecting at least one SARS-CoV-2 nucleocapsid protein (e.g., antigen) in a sample. In some aspects, the technology relates to analytical devices that are suitable for use in the home, clinic, or hospital, and that are intended to give an analytical result that is rapid with minimum degree of skill and involvement from the user. In some aspects, use of the devices described herein involves methods in which a user performs a sequence of operations to provide an observable test result.

In some aspects, also provided is a test device comprising a reagent-impregnated test strip to provide a specific binding assay, e.g., an immunoassay. In some embodiments, a sample is applied to one portion of the test strip and is allowed to permeate through the strip material, usually with the aid of an eluting solvent such as water and/or a suitable buffer (e.g., an extraction buffer optionally comprising a detergent). In so doing, the sample progresses into or through a detection zone in the test strip wherein a first specific binding partner (e.g., an antibody or a fragment thereof and/or a recombinant antigen) for an analyte (e.g., SARS-CoV-2 nucleocapsid protein) suspected of being in the sample is immobilized. Analyte present in the sample can therefore become bound within the detection zone. The extent to which the analyte becomes bound in that zone can be determined with the aid of labelled reagents that can also be incorporated in the test strip or applied thereto subsequently.

In some aspects, the analytical test device comprises a hollow casing constructed of moisture-impervious solid material containing a dry porous carrier that communicates directly or indirectly with the exterior of the casing such that a liquid test sample can be applied to the porous carrier. In some aspects, the device also comprises a labelled specific binding partner for an analyte and the labelled specific binding partner is freely mobile within the porous carrier when in the moist state. In some aspects, the device comprises unlabeled specific binding partner for the same analyte and the unlabeled reagent is permanently immobilized in a detection zone on the carrier material and is therefore not mobile in the moist state. The relative positioning of the labelled reagent and detection zone being such that liquid sample applied to the device can pick up labelled reagent and thereafter permeate into the detection zone and the device provides the extent (if any) to which the labelled reagent becomes in the detection zone to be observed.

Another aspect relates to a device that comprises a porous solid phase material carrying in a first zone a labelled reagent that is retained in the first zone while the porous material is in the dry state but is free to migrate through the porous material when the porous material is moistened, for example, by the application of an aqueous liquid sample suspected of containing the analyte. In some embodiments, the porous material comprises in a second zone, which is spatially distinct from the first zone, an unlabeled specific binding partner (e.g., a recombinant antigen and//or antibody) having specificity for the analyte and which is capable of participating with the labelled reagent in either a “sandwich” or a “competition” reaction. The unlabeled specific binding partner is firmly immobilized on the porous material such that it is not free to migrate when the porous material is in the moist state. In some aspects, the labelled reagent is a specific binding partner (e.g., a second specific binding partner) for the analyte. The labelled reagent, the analyte (if present), and the immobilized unlabeled specific binding partner (e.g., first specific binding partner) have specificities for different epitopes on the analyte and cooperate together in a reaction.

In some embodiments, a device as described herein is contacted with an aqueous liquid sample suspected of containing the analyte, such that the sample permeates by capillary action through the porous solid phase material via the first zone into the second zone and the labelled reagent migrates therewith from the first zone to the second zone, the presence of analyte in the sample being determined.

Examples of lateral flow assays that can be used in the present disclosure include, for example, Panbio®, Binax® and BinaxNOW® (Alere, Abbott Park, Ill.).

### 6. SAMPLES AND CONTROLS

a. Test or Biological Sample

As used herein, “sample”, “test sample”, “biological sample” refer to fluid sample containing or suspected of containing at least one SARS-CoV-2 nucleocapsid protein. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing at least one SARS-CoV-2 nucleocapsid protein may be assayed directly. In a particular example, the source of a SARS-CoV-2 nucleocapsid protein is a mammalian (e.g., human) bodily substance (e.g., bodily fluid, blood such as whole blood (including, for example, capillary blood, venous blood, etc.), serum, plasma, urine, saliva, sweat, sputum, semen, mucus, lacrimal fluid, lymph fluid, amniotic fluid, interstitial fluid, lower respiratory specimens such as, but not limited to, sputum, endotracheal aspirate or bronchoalveolar lavage, cerebrospinal fluid, feces, tissue, organ, one or more dried blood spots, or the like). Tissues may include, but are not limited to oropharyngeal specimens, nasopharyngeal specimens, skeletal muscle tissue, liver tissue, lung tissue, kidney tissue, myocardial tissue, brain tissue, bone marrow, cervix tissue, skin, etc. The sample may be a liquid sample or a liquid extract of a solid sample. In certain cases, the source of the sample may be an organ or tissue, such as a biopsy sample, which may be solubilized by tissue disintegration/cell lysis. Additionally, the sample can be a nasopharyngeal or oropharyngeal sample obtained using one or more swabs that, once obtained, is placed in a sterile tube containing a virus transport media (VTM) or universal transport media (UTM), for testing.

A wide range of volumes of the fluid sample may be analyzed. In a few exemplary embodiments, the sample volume may be about 0.5 nL, about 1 nL, about 3 nL, about 0.01 μL, about 0.1 μL, about 1 μL, about 5 μL, about 10 μL, about 100 μL, about 1 mL, about 5 mL, about 10 mL, or the like. In some cases, the volume of the fluid sample is between about 0.01 μL, and about 10 mL, between about 0.01 μL, and about 1 mL, between about 0.01 μL and about 100 μL, or between about 0.1 μL, and about 10 μL.

In some cases, the fluid sample may be diluted prior to use in an assay. For example, in embodiments where the source of a SARS-CoV-2 nucleocapsid protein is a human body fluid (e.g., blood, serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 100-fold, or greater, prior to use. In other cases, the fluid sample is not diluted prior to use in an assay. In some aspects, the diluent may optionally contain an antibody, such as an IgG antibody that is added to remove any IgG antibodies from the sample.

In some cases, the sample may undergo pre-analytical processing or pre-treatment. Pre-analytical processing may offer additional functionality such as nonspecific protein removal and/or effective yet cheaply implementable mixing functionality. General methods of pre-analytical processing may include the use of electrokinetic trapping, AC electrokinetics, surface acoustic waves, isotachophoresis, dielectrophoresis, electrophoresis, or other pre-concentration techniques known in the art.

In some cases, pre-treatment may involve adding an antibody, such as an IgG and/or IgM antibody to the biological sample prior to the addition of the at least one first specific binding partner and/or at least one second specific binding partner.

In some cases, the fluid sample may be concentrated prior to use in an assay. For example, in embodiments where the source of a SARS-CoV-2 nucleocapsid protein is a human body fluid (e.g., blood, serum), the fluid may be concentrated by precipitation, evaporation, filtration, centrifugation, or a combination thereof. A fluid sample may be concentrated about 1-fold, about 2-fold, about 3-fold, about 4-fold, about 5-fold, about 6-fold, about 10-fold, about 100-fold, or greater, prior to use.

b. Controls and Calibrators

It may be desirable to include a control (such as a positive and/or negative control, which are well known in the art). For example, a positive control can be purified from in vivo or any recombinant SARS-CoV-2 nucleocapsid protein or variant thereof that binds to the first specific binding partner (e.g., anti-SARS-CoV antibody, anti-SARS-CoV-2 antibody or any fragment thereof). In some aspects, the positive control can be the full-length SARS-CoV-2 nucleocapsid protein (such as a human SARS-CoV-2 nucleocapsid protein, such as shown in SEQ ID NO:1). Alternatively, the positive control can be a fragment or variant of the full-length SARS-CoV-2 nucleocapsid protein (such as a human SARS-CoV-2 nucleocapsid protein, such as shown in SEQ ID NO:1). In some aspects, the control can be a cell culture derived virus (that may or may not have been purified, e.g., lysates or cell culture medium). Examples of come cell culture derived viruses that can be used include Vero cells that have been infected with SARS-CoV or SARS-CoV-2.

The control may be analyzed separately from, or concurrently with, the sample from the subject as described above. The results obtained from the subject sample can be compared to the results or information obtained from the control sample. Standard curves may be provided or developed with use of the calibrators and controls, with which assay results for the sample may be compared. Such standard curves typically present levels of marker as a function of assay units (i.e., fluorescent signal intensity, if a fluorescent label is used).

It may also be desirable to include one or more calibrators for use in calibrating of any automated or semi-automated system for which the methods and kits described herein are adapted for use. The use of calibrators in such systems is well known in the art. For example, one or more calibrators can include the full-length SARS-CoV-2 nucleocapsid protein (such as a human SARS-CoV-2 nucleocapsid protein, such as shown in SEQ ID NO:1). Alternatively, the calibrator can be a fragment or variant of the full-length SARS-CoV-2 nucleocapsid protein (such as a human SARS-CoV-2 nucleocapsid protein, such as shown in SEQ ID NO:1).

In some aspects, the calibrator can be a cell culture derived virus (that may or may not have been purified, e.g., lysates or cell culture medium). Examples of come cell culture derived viruses that can be used include Vero cells that have been infected with SARS-CoV or SARS-CoV-2.

The calibrator is optionally, part of a series of calibrators in which each of the calibrators differs from the other calibrators in the series by the concentration of at least one SARS-CoV-2 nucleocapsid protein (such as a human SARS-CoV-2 nucleocapsid protein, such as shown in SEQ ID NO:1).

### 7. KIT

Provided herein is a kit, which may be used in the methods described herein for assaying or assessing a test sample for at least one SARS-CoV-2 nucleocapsid protein (e.g., antigen). The kit comprises at least one component for assaying the test sample for a SARS-CoV-2 nucleocapsid protein as well as instructions for assaying the test sample at least one SARS-CoV-2 nucleocapsid protein. For example, the kit can comprise instructions for assaying the test sample for a a SARS-CoV-2 nucleocapsid protein using an immunoassay, e.g., chemiluminescent microparticle immunoassay. Instructions included in kits can be affixed to packaging material, can be included as a package insert, or can be viewed or downloaded from a particular website that is recited as part of the kit packaging or inserted materials. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The at least one component for assaying the test sample for a SARS-CoV-2 nucleocapsid protein may include at least one composition comprising one or more anti-SARS-CoV antibodies, anti-SARS-CoV-2 antibodies or any fragments thereof that specifically bind to a SARS-CoV-2 nucleocapsid protein as described previously herein. The kit further can include anti-SARS-CoV or anti-SARS-CoV-2 antigens purified from in vivo or recombinant antigens, e.g., for use as calibrators or controls, or optionally, these can be provided separately.

Alternatively or additionally, the kit can comprise a calibrator or control, e.g., purified, and optionally frozen or lyophilized, as described previously herein, and/or at least one container (e.g., tube, microtiter plates or strips, which can be already coated with an anti-SARS-CoV antibody, anti-SARS-CoV-2 antibody or fragment thereof, and/or any recombinant antigen (e.g., as calibrator or control) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve.

The kit may further comprise reference standards for quantifying a SARS-CoV-2 nucleocapsid protein. The reference standards may be employed to establish standard curves for interpolation and/or extrapolation of a SARS-CoV-2 recombinant protein concentration.

Any antibodies, such as recombinant antibodies and/or any recombinant antigens, which are provided in the kit, can incorporate a detectable label, such as a fluorophore, radioactive moiety, enzyme, biotin/avidin label, chromophore, chemiluminescent label, or the like, or the kit can include reagents for labeling the antibodies and/or reagents for detecting the SARS-CoV-2 antigen (e.g., detection antibodies) and/or for labeling the analytes (e.g., SARS-CoV-2 nucleocapsid protein) or reagents for detecting the analyte (e.g., SARS-CoV-2 nucleocapsid protein). The antigens (e.g., polypeptides), antibodies, calibrators, and/or controls can be provided in separate containers or pre-dispensed into an appropriate assay format, for example, into microtiter plates,

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays,

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents or extraction buffers), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine, whole blood, plasma, or serum sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

The kit can also include one or more sample collection/acquisition instruments for assisting with obtaining a test sample (e.g., microsampling devices, micro-needles, or other minimally invasive pain-free blood collection methods; blood collection tube(s); lancets; capillary blood collection tubes; other single fingertip-prick blood collection methods; buccal swabs, nasal/throat swabs; 16-gauge or other size needle, surgical knife or laser (e.g., particularly hand-held), syringes, sterile container, or canula, for obtaining, storing, or aspirating tissue samples).

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution. If desired, the kit can contain a solid phase, such as a magnetic particle, bead, test tube, microtiter plate, cuvette, membrane, scaffolding molecule, film, filter paper, disc, or chip.

If desired, the kit can further comprise one or more components, alone or in further combination with instructions, for assaying the test sample for another analyte, which can be a biomarker, such as a biomarker of SARS-CoV-2 infection.

### 8. ADAPTATION OF KIT AND METHOD

The kit (or components thereof), as well as the method for detecting the presence or determining the amount or level or concentration of a SARS-CoV-2 nucleocapsid protein in a test sample by an immunoassay as described herein, can be adapted for use in a variety of automated and semi-automated systems or platforms (including those wherein the solid phase comprises a microparticle), as described, e.g., U.S. Pat. No. 5,063,081, U.S. Patent Application Publication Nos. 2003/0170881, 2004/0018577, 2005/0054078, and 2006/0160164 and as commercially marketed e.g., by Abbott Laboratories (Abbott Park, Ill.) as Abbott Point of Care (i-STAT® or i-STAT® Alinity, Abbott Laboratories) as well as those described in U.S. Pat. Nos. 5,089,424 and 5,006,309, and as commercially marketed, e.g., by Abbott Laboratories (Abbott Park, Ill.) as ARCHITECT® or the series of Abbott Alinity devices. Such systems include one or more devices and/or components that can be used to detect one or more labels in the resulting complexes formed in the methods described previously herein.

Some of the differences between an automated or semi-automated system as compared to a non-automated system include the substrate to which the first specific binding partner (e.g., recombinant antigen or capture reagent) is attached, and the length and timing of the capture, detection, and/or any optional wash steps. Whereas a non-automated format may require a relatively longer incubation time with test sample and capture reagent (e.g., about 2 hours), an automated or semi-automated format (e.g., ARCHITECT®, Alinity, and any successor platform, Abbott Laboratories) may have a relatively shorter incubation time (e.g., approximately 18 minutes for ARCHITECT®). Similarly, whereas a non-automated format may incubate a detection antibody such as the conjugate reagent for a relatively longer incubation time (e.g., about 2 hours), an automated or semi-automated format (e.g., ARCHITECT®, Alinity, and any successor platform) may have a relatively shorter incubation time (e.g., approximately 4 minutes for the ARCHITECT®, Alinity, and any successor platform).

Other platforms available from Abbott Laboratories include, but are not limited to, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits, and kit components can be employed in other formats, for example, on electrochemical or other hand-held or point-of-care assay systems. As mentioned previously, the present disclosure is, for example, applicable to the commercial Abbott Point of Care (i-STAT® ori-STAT® Alinity, Abbott Laboratories) electrochemical immunoassay system that performs sandwich immunoassays. Immunosensors and their methods of manufacture and operation in single-use test devices are described, for example in, U.S. Pat. No. 5,063,081, U.S. Patent App. Publication Nos. 2003/0170881, 2004/0018577, 2005/0054078, and 2006/0160164, which are incorporated in their entireties by reference for their teachings regarding same.

In particular, with regard to the adaptation of an assay to the i-STAT® or i-STAT® Alinity system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an i-STAT® or i-STAT ®Alinity cartridge with a fluidics format suitable for immunoassay. On a portion of the silicon chip, there is a specific binding partner for a SARS-CoV-2 nucleocapsid protein, such as at least one specific binding partner as described herein (e.g., anti-SARS-CoV antibody, anti-SARS-CoV-2 antibody, or fragment thereof) or one or more SARS-CoV or SARS-CoV-2 DVD-Igs (or a fragment thereof, a variant thereof, or a fragment of a variant thereof, that can bind a SARS-CoV-2 nucleocapsid protein, which can be detectably labeled. Within the fluid pouch of the cartridge is an aqueous reagent that includes p-aminophenol phosphate.

In operation, a sample from a subject suspected of having or being exposed to SARS-CoV-2 is added to the holding chamber of the test cartridge, and the cartridge is inserted into the i-STAT® or i-STAT® Alinity reader. A pump element within the cartridge pushes the sample into a conduit containing the chip. The sample is brought into contact with the sensors allowing the enzyme conjugate to dissolve into the sample. The sample is oscillated across the sensors to promote formation of the sandwich of approximately 2-12 minutes. In the penultimate step of the assay, the sample is pushed into a waste chamber and wash fluid, containing a substrate for the alkaline phosphatase enzyme, is used to wash excess enzyme conjugate and sample off the sensor chip. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of anti-β-coronavirus antibody in the sample by means of an embedded algorithm and factory-determined calibration curve. Adaptation of a cartridge for multiplex use, such as used for i-STAT® or i-STAT® Alinity, has been described in the patent literature, such as for example, U.S. Pat. No. 6,438,498, the contents of which are herein incorporated by reference.

The methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT® or Alinity conjugate diluent employed in certain kits (Abbott Laboratories, Abbott Park, Ill.) and containing 2-(N-morpholino)ethanesulfonic acid (MES), a salt, a protein blocker, an antimicrobial agent, and a detergent. An exemplary calibrator diluent is ARCHITECT® or Alinity human calibrator diluent employed in certain kits (Abbott Laboratories, Abbott Park, Ill.), which comprises a buffer containing MES, other salt, a protein blocker, and an antimicrobial agent. Additionally, as described in U.S. Patent Application No. 61/142,048 filed Dec. 31, 2008, improved signal generation may be obtained, e.g., in an i-STAT® or i-STAT® Alinity cartridge format, using a nucleic acid sequence linked to the signal antibody as a signal amplifier.

### 9. ANALYSIS AND INTREPRETATION OF RESULTS

The results obtained using the methods of the present disclosure (e.g., detecting the presence of at least one SARS-CoV-2 nucleocapsid protein (e.g., antigen) or determining the amount, level or concentration of at least one one SARS-CoV-2 nucleocapsid protein in a biological sample) can be analyzed and interpreted individually or in combination with other any other results obtained prior to, during or after the results of the methods of the present disclosure are performed. The nature of the other results analyzed and interpreted with the results of the present disclosure are changeable. For example, in one aspect, if the methods of present disclosure are used to detect the presence of or determine the amount, level or concentration of at least one one SARS-CoV-2 nucleocapsid protein in a biological sample, these results can be used alone or in combination with concurrently, previously, or later obtained results relating to detecting the presence of or determining the amount, level or concentration of at least one anti-SARS-CoV-2 IgM and/or IgG antibody in a biological sample obtained from the same subject.

The results relating to detecting the presence of or determining the amount, level or concentration of at least one anti-SARS-CoV-2 IgM and/or IgG antibody in a biological sample obtained from the subject may have been obtained at the same time, or minutes, hours or days before or after the results relating to detecting the presence of or determining the amount, level or concentration of at least one SARS-CoV-2 nucleocapsid protein (e.g., antigen) in the sample were obtained. Analyzing the combined results regarding the presence of or amount, level, or concentration of at least one SARS-CoV-2 nucleocapsid protein and/or any SARS-CoV-2 IgM and/or IgG antibody levels can guide treatment and/or monitoring decisions to be made by a clinician.

The present disclosure has multiple aspects, illustrated by the following non-limited examples.

### 10. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

### Example 1: Improved Sandwich Assay

Human SARS-CoV-2 antigen was assessed using a sandwich assay on the ARCHITECT® automated analyzer (Abbott Laboratories, Abbott Park, Ill.) utilizing the following reagents: (1) Capture reagent: Dynabeads® M-270 streptavidin microparticles (Thermofisher Scientific, Waltham, Mass.) coated with scFv antibody N18 (described in Zhao, et al., Microbes and Infection, 9:1026-1033 (2007), the contents of which are herein incorporated by reference); (2) Detection reagent: Biotinylated human monoclonal antibody CR3009 (described in van den Brink et al., Journal of Vir. 79(3):1635-1644 (Feb. 2005), the contents of which are herein incorporated by reference); (3) a diluent solution containing: (i) no poly-L-lysine hydrobromide (molecular weight of about 1,000 to about 5,000 daltons) (“PLL”); (ii) 1 ng/mL poly-L-lysine hydrobromide (molecular weight of about 1,000 to about 5,000 daltons); (iii) 10 ng/mL poly-L-lysine hydrobromide (molecular weight of about 1,000 to about 5,000 daltons); (iv) 50 ng/mL poly-L-lysine hydrobromide (molecular weight of about 1,000 to about 5,000 daltons); or (v) 100 ng/mL poly-L-lysine hydrobromide (molecular weight of about 1,000 to about 5,000 daltons); and (4) a calibrator comprising the full-length (amino acids 1-419) human SARS-CoV-2 nucleocapsid protein.

The capture reagent, detection reagent, and diluent solutions containing 0 ng/mL PLL, 1 ng/mL PLL, 10 ng/mL PLL, 50 ng/mL PLL, or 100 ng/mL of PLL were combined with (1) nasopharyngeal swab in viral transport medium or buffer; (2) nasal swab in in viral transport medium or buffer; or (3) saliva with or without viral transport medium or buffer. After an 18 minute incubation, the microparticles were washed and signal (S) detected from the N18-SARS-CoV-2 nucleocapsid protein-CR3009 complex, expressed in relative light units (RLU), as shown in Table A. The corresponding signal (S) to noise (N) ratio for each of the four different concentrations of poly-L-lysine tested are shown in Table B.

TABLE A

| | Biotin Incorporation | | Biotin Incorporation | |
|---|---|---|---|---|
| Poly-L-Lysine | Ratio: 3.4 | | Ratio: 6.4 | |
| concentration | No PLL | PLL | No PLL | PLL |
| 0 ng/mL | 11674 | 124806 | 14171 | 126614 |
| 1 ng/mL | 22185 | 317028 | 24242 | 297020 |
| 10 ng/mL | 92303 | 799763 | 104347 | 738801 |
| 50 ng/mL | 418549 | 1314532 | 464428 | 1221717 |
| 100 ng/mL | 746888 | 1444008 | 784920 | 1401570 |

TABLE B

| | Biotin Incorporation | | Biotin Incorporation | |
|---|---|---|---|---|
| Poly-L-Lysine | Ratio: 3.4 | | Ratio: 6.4 | |
| concentration | No PLL | PLL | No PLL | PLL |
| 1 ng/mL | 1.90 | 2.54 | 1.71 | 2.35 |
| 10 ng/mL | 7.91 | 6.41 | 7.36 | 5.84 |
| 50 ng/mL | 35.85 | 10.53 | 32.77 | 9.65 |
| 100 ng/mL | 63.98 | 11.57 | 55.39 | 11.07 |

As shown in Table B, 1 ng/mL of PLL improved the sensitivity of the method by from at least about 34% (i.e., biotin incorporation ratio of 3.4) to about 37% (i.e., biotin incorporation ratio of 6.4).

Similar results as shown in Table A and B demonstrating an improvement in sensitivity were observed for SARS-CoV-2 nucleocapsid antigen assays performed using other anti-SARS-CoV antibodies as capture and detection reagents. Specifically, improvements in the sensitivity of from at least about 19% to about 280% in the above method were observed using PLL at various concentrations of 1 ng/mL, 10 ng/mL, 50 ng/mL and 100 ng/mL.

Additionally, poly-D-lysine hydrobromide (PDL), polyethylenimines having a molecular weight of 800 daltons (PEI800) and polyethylenimines having a molecular weight of 1300 daltons (PEI1300) were also shown to increase the sensitivity of the assay for nucleocapsid antigen detection (with PDL performing better than PEI800, which in turn performed better than PEI1300) but not nearly as much as PLL. In contrast, cetyltrimethylammonium bromide (CTAB), dodecyl trimethylammonium bromide (DTAB), 3-(N,N-dimethymyristylammonio)propanesulfonate (SB3-14), Pluronic 17R4, and Pluracare 1307 were not found to increase sensitivity.

### Example 2—SARS-CoV-2 Variants

SARS-CoV-2 B.1.1.7 strain first identified in the United Kingdom is of utmost concern for evaluation due to the observed link between increased transmissibility and spike gene mutation. While spike gene mutations primarily define the B.1.1.7 lineage, the presence of additional mutations throughout the genome warrant further examination for potential impact on diagnostic lateral flow assay performance.

Initial in silico examination of B.1.1.7 lineage sequences in GISAID (N=1787 accessed 12/21/2020) revealed no lineage-defining mutations of concern for the lateral flow sandwich assays described herein (e.g., lateral flow antigen assay 1 and lateral flow antigen assay 2). A virus culture (BEI NR-54011, EPI_ISL_751801) was heat inactivated at 65° C. for 30 minutes and tested in a dilution series. Multiple dilutions were detected with the assays in the expected ranges previously observed with other strains (Table C). The results confirmed the in silico prediction that these lateral flow antigen assays can reliably detect the B.1.1.7 strain.

TABLE C
Rapid antigen lateral flow sandwich assay results with
B.1.1.7 virus culture dilutions

| | | | Lateral flow | Lateral flow |
|---|---|---|---|---|
| | | | antigen assay | antigen assay |
| | | | 1 | 2 |
| Dilution | Log GE/test* | TCID₅₀/test | Result | Result |
| Neat | 7.56 | 560 | 1/1 Positive | 1/1 Positive |
| 1 | 6.56 | 56 | 4/4 Positive | 3/3 Positive |
| 2 | 6.26 | 28 | 4/4 Positive | 3/3 Positive |
| 3 | 5.96 | 14 | 3/4 Positive | 3/4 Positive |

*Log genome equivalents (GE)/test were calculated from a standard curve with an R2 value of 0.99

The B.1.351 lineage was first identified in South Africa and has since spread to over a dozen countries, with initial reports indicating this variant may escape neutralizing antibodies. The unique mutation profile of the B.1.351 lineage is primarily defined by spike gene mutations K417N, E484K, and N501Y, however, the presence of additional mutations throughout the genome may impact the performance of a variety of diagnostic lateral flow assays.

Initial in silico examination of B.1.351 lineage sequences in GISAID (N=195, as accessed Dec. 27, 2020) did not reveal any lineage-defining mutations of concern for the performance of the methods described herein. Two virus cultures (BEI NR-54008, NR-54009) were heat inactivated, tested in dilution series, and detected in the ranges previously observed with other strains (Table D). These results confirmed the in silico prediction that the can reliably detect the B.1.351 lineage.

TABLE D
Rapid antigen lateral flow sandwich assay results

| | NR-54008 | | | NR-54009 | | |
|---|---|---|---|---|---|---|
| | | Lateral | Lateral | | | Lateral |
| | | flow | flow | | Lateral flow | flow |
| | Log GE*/ | antigen | antigen | Log | antigen | antigen |
| | test | assay 1 | assay 2 Ag | GE/test | assay 1 | assay 2 |
| 1160 | 6.25 | 3/3 | 3/3 | 5.98 | 3/3 | 3/3 |
| | | detected | detected | | detected | detected |
| 928 | 6.15 | 3/3 | 3/3 | 5.88 | 3/3 | 3/3 |
| | | detected | detected | | detected | detected |
| 560 | 5.93 | 6/6 | 3/3 | 5.66 | 6/6 | 3/3 |
| | | detected | detected | | detected | detected |
| 56 | 4.93 | 0/3 | 0/3 | 4.66 | 3/3 | 0/3 |
| | | detected | detected | | detected | detected |

*Log genome equivalents (GE)/mL were calculated from a GE/ml standard curve with an R² value of 0.99 and a unit conversion plot of TCID50/mL vs GE/mL with an R² value of 0.99

The present disclosure has multiple aspects, illustrated by the following non-limiting examples.

It is understood that the foregoing detailed description and accompanying examples are merely illustrative and are not to be taken as limitations upon the scope, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use, may be made without departing from the spirit and scope thereof.

For reasons of completeness, various aspects are set out in the following numbered clauses:

Clause 1. In an improvement of a method of detecting a presence or determining an amount of a SARS-CoV-2 nucleocapsid protein in a biological sample, wherein the method comprises detecting at least one complex comprising a first specific binding partner, said sample SARS-CoV-2 nucleocapsid protein, and a second specific binding partner comprising at least one detectable label and further wherein the first specific binding partner, second specific binding partner, or the first specific binding partner and the second specific binding partner comprise at least one anti-SARS-CoV antibody, anti-SARS-CoV-2 antibody, or fragment thereof that specifically binds to said sample SARS-CoV-2 nucleocapsid protein, the improvement comprising allowing the complex to form in the presence of at least one polycation having a molecular weight of at least about 500 daltons or greater prior to assessing the signal from the complex, wherein the amount of detectable signal from the detectable label in the complex indicates the presence or amount of SARS-CoV-2 nucleocapsid protein in the sample.

Clause 2. The improvement of clause 1, wherein the biological sample is whole blood, serum, plasma, saliva, an oropharyngeal specimen, or a nasopharyngeal specimen.

Clause 3. The improvement of clauses 1 or 2, wherein the first specific binding partner comprises at least one anti-SARS-CoV antibody, anti-SARS-CoV-2 antibody, or fragment thereof, or the first specific binding partner and the second specific binding partner each comprise at least one anti-SARS-CoV antibody, anti-SARS-CoV-2 antibody or fragment thereof.

Clause 4. The improvement of any of clauses 1-3, wherein the polycation is at least one polylysine, at least one polyornithine, at least one poly-L-histidine, at least one poly-L-arginine, at least one polyethylenimine, at least one DEAE-Dextran, or combinations thereof

Clause 5. The improvement of clause 4, wherein the (i) the polylysine is poly-L-lysine hydrobromide, poly-D-lysine hydrobromide, poly-L-lysine hydrochloride, poly-L-lysine trifluoroacetate, poly(lysine, alanine) 3:1 hydrobromide, poly(lysine, arginine) 2:1 hydrobromide, poly(lysine, alanine) 1:1 hydrobromide, or poly(lysine, tryptophan) 1:4 hydrobromide; (ii) the polyornithine is poly-L-ornithine hydrobromide or poly-DL-ornithine hydrobromide; (iii) the poly-L-histidine is poly-L-histidine hydrobromide; and (iv) the poly-L-arginine is poly-L-arginine hydrochloride or poly-L-arginine hydrobromide.

Clause 6. The improvement of any of clauses 1-5, wherein the method is selected from the group consisting of an immunoassay, a clinical chemistry assay, a point-of-care assay, and a lateral flow assay.

Clause 7. The improvement of any of clauses 1-6, wherein the method is performed using single molecule detection.

Clause 8. The improvement of any of clauses 1-7, wherein the method is adapted for use in an automated system or a semi-automated system.

Clause 9. A method of detecting a presence or determining an amount of a SARS-CoV-2 nucleocapsid protein in a biological sample in a subject, the method comprising:
c) contacting at least one biological sample from the subject, either simultaneously or sequentially, in any order, with:
   at least one first specific binding partner comprising at least one anti-SARS-CoV antibody, anti-SARS-CoV-2 antibody, or fragment thereof thereof that specifically binds to at least one SARS-CoV-2 nucleocapsid protein in the sample,
   at least one second specific binding partner comprising at least one detectable label, thereby producing one or more complexes comprising the first binding member-SARS-CoV-2 nucleocapsid protein-second specific binding partner, and
   at least one polycation having a molecular weight of at least about 500 daltons or greater; and
d) assessing a signal from the one or more complexes, wherein the amount of detectable signal from the detectable label indicates the presence or amount of SARS-CoV-2 nucleocapsid protein in the sample.

Clause 10. The method of clause 9, wherein the biological sample is whole blood, serum, plasma, saliva, an oropharyngeal specimen, or a nasopharyngeal specimen.

Clause 11. The method of clause 9 or clause 10, wherein the first specific binding partner comprises at least one anti-SARS-CoV antibody, anti-SARS-CoV-2 antibody, or fragment thereof, or the first specific binding partner and the second specific binding partner each comprise at least one anti-SARS-CoV antibody, anti-SARS-CoV-2 antibody, or fragment thereof.

Clause 12. The method of any of clauses 9-11, wherein the polycation is at least one polylysine, at least one polyornithine, at least one poly-L-histidine, at least one poly-L-arginine, at least one polyethylenimines, at least one DEAE-Dextran, or combinations thereof.

Clause 13. The method of clause 12, wherein the (i) the polylysine is poly-L-lysine hydrobromide, poly-D-lysine hydrobromide, poly-L-lysine hydrochloride, poly-L-lysine trifluoroacetate, poly(lysine, alanine) 3:1 hydrobromide, poly(lysine, arginine) 2:1 hydrobromide, poly(lysine, alanine) 1:1 hydrobromide, or poly(lysine, tryptophan) 1:4 hydrobromide; (ii) the polyornithine is poly-L-ornithine hydrobromide or poly-DL-ornithine hydrobromide; (iii) the poly-L-histidine is poly-L-histidine hydrobromide; and (iv) the poly-L-arginine is poly-L-arginine hydrochloride or poly-L-arginine hydrobromide.

Clause 14. The method of any of clauses 9-13, wherein the method is selected from the group consisting of an immunoassay, a clinical chemistry assay, a point-of-care assay, and a lateral flow assay.

Clause 15. The method of any of clauses 9-14, wherein the method is performed using single molecule detection.

Clause 16. The method of any of clauses 9-15, wherein the method is adapted for use in an automated system or a semi-automated system.

Clause 17. A kit for performing the method of clause 9, wherein the kit comprises:
d. at least one specific binding partner comprising at least one anti-SARS-CoV antibody, anti-SARS-CoV-2 antibody, or fragment thereof that specifically binds to at least one SARS-CoV-2 nucleocapsid protein;
e. at least one second specific binding partner comprising at least one detectable label; and
f. at least one polycation having a molecular weight of at least about 500 daltons or greater.

Clause 18. The kit of clause 17, wherein the kit further comprises, or is configured to be used with at least one calibrator reagent, at least one control reagent, or at least one calibrator reagent and at least one control reagent.

Clause 19. The kit of any of clause 17 or clause 18, wherein the kit further comprises at least one solid support.

Clause 20. The kit of any of clauses 17-19, wherein the polycation is at least one polylysine, at least one polyornithine, at least one poly-L-histidine, at least one poly-L-arginine, at least one polyethylenimines, at least one DEAE-Dextran, or combinations thereof.

Clause 21. The kit of clause 20, wherein the (i) the polylysine is poly-L-lysine hydrobromide, poly-D-lysine hydrobromide, poly-L-lysine hydrochloride, poly-L-lysine trifluoroacetate, poly(lysine, alanine) 3:1 hydrobromide, poly(lysine, arginine) 2:1 hydrobromide, poly(lysine, alanine) 1:1 hydrobromide, or poly(lysine, tryptophan) 1:4 hydrobromide; (ii) the polyornithine is poly-L-ornithine hydrobromide or poly-DL-ornithine hydrobromide; (iii) the poly-L-histidine is poly-L-histidine hydrobromide; and (iv) the poly-L-arginine is poly-L-arginine hydrochloride or poly-L-arginine hydrobromide.

Clause 22. The kit of any of clauses 17-21, wherein the kit is adapted for use with an automated or semi-automated system.

Clause 23. A system for detecting a presence or determining an amount of a SARS-CoV-2 nucleocapsid protein in a biological sample in a subject, the method comprising:
at least one first specific binding partner comprising at least anti-SARS-CoV antibody, anti-SARS-CoV-2 antibody, or fragment thereof that specifically binds to at least one SARS-CoV-2 nucleocapsid protein in the sample;
at least one second specific binding partner comprising at least one detectable label, thereby producing one or more complexes comprising the first binding member-SARS-CoV-2 nucleocapsid protein-second specific binding partner;
at least one polycation having a molecular weight of at least about 500 daltons or greater; and
at least one device for detecting the at least one label from the complex.

Clause 24. The system of clause 23, wherein the device for detecting the label from the complex is automated or semi-automated.

Clause 25. The system of clause 23 or clause 24, wherein the polycation is at least one polylysine, at least one polyornithine, at least one poly-L-histidine, at least one poly-L-arginine, at least one polyethylenimines, at least one DEAE-Dextran, or combinations thereof.

Clause 26. The system of clause 25, wherein the (i) the polylysine is poly-L-lysine hydrobromide, poly-D-lysine hydrobromide, poly-L-lysine hydrochloride, poly-L-lysine trifluoroacetate, poly(lysine, alanine) 3:1 hydrobromide, poly(lysine, arginine) 2:1 hydrobromide, poly(lysine, alanine) 1:1 hydrobromide, or poly(lysine, tryptophan) 1:4 hydrobromide; (ii) the polyornithine is poly-L-ornithine hydrobromide or poly-DL-ornithine hydrobromide; (iii) the poly-L-histidine is poly-L-histidine hydrobromide; and (iv) the poly-L-arginine is poly-L-arginine hydrochloride or poly-L-arginine hydrobromide.

Clause 27. The method of any of clauses 1-8 wherein the improvement increases sensitivity by at least about 5%, at least about 10%, at least about 15%, at least about 20%, at least about 25%, at least about 30%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 95%, at least about 100%, at least about 110%, at least about 120%, at least about 125%, at least about 130%, at least about 140%, at least about 150%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% when compared to methods which do not use or employ at least one polycation.

Clause 28. The method of any of clauses 9-16 wherein the improvement increases sensitivity by at least about 5%, at least about 10%, at least about 15%, at least about 20%, at least about 25%, at least about 30%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 95%, at least about 100%, at least about 110%, at least about 120%, at least about 125%, at least about 130%, at least about 140%, at least about 150%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% when compared to methods which do not use or employ at least one polycation.

What is claimed is:

1. In an improvement of a method of detecting a presence or determining an amount of a SARS-CoV-2 nucleocapsid protein in a biological sample, wherein the method comprises detecting at least one complex comprising a first specific binding partner, said sample SARS-CoV-2 nucleocapsid protein, and a second specific binding partner comprising at least one detectable label and further wherein the first specific binding partner, second specific binding partner, or the first specific binding partner and the second specific binding partner comprise at least one anti-SARS-CoV antibody, anti-SARS-CoV-2 antibody, or fragment thereof that specifically binds to said sample SARS-CoV-2 nucleocapsid protein, the improvement comprising allowing the complex to form in the presence of at least one polycation having a molecular weight of at least about 500 daltons or greater prior to assessing the signal from the complex, wherein the amount of detectable signal from the detectable label in the complex indicates the presence or amount of SARS-CoV-2 nucleocapsid protein in the sample.

2. The improvement of claim 1, wherein the biological sample is whole blood, serum, plasma, saliva, an oropharyngeal specimen, or a nasopharyngeal specimen.

3. The improvement of claim 1, wherein the first specific binding partner comprises at least one anti-SARS-CoV antibody, anti-SARS-CoV-2 antibody, or fragment thereof, or the first specific binding partner and the second specific binding partner each comprise at least one anti-SARS-CoV antibody, anti-SARS-CoV-2 antibody or fragment thereof.

4. The improvement of claim 1, wherein the polycation is at least one polylysine, at least one polyornithine, at least one poly-L-histidine, at least one poly-L-arginine, at least one polyethylenimine, at least one DEAE-Dextran, or combinations thereof.

5. The improvement of claim 4, wherein the (i) the polylysine is poly-L-lysine hydrobromide, poly-D-lysine hydrobromide, poly-L-lysine hydrochloride, poly-L-lysine trifluoroacetate, poly(lysine, alanine) 3:1 hydrobromide, poly(lysine, arginine) 2:1 hydrobromide, poly(lysine, alanine) 1:1 hydrobromide, or poly(lysine, tryptophan) 1:4 hydrobromide; (ii) the polyornithine is poly-L-ornithine hydrobromide or poly-DL-ornithine hydrobromide; (iii) the poly-L-histidine is poly-L-histidine hydrobromide; and (iv) the poly-L-arginine is poly-L-arginine hydrochloride or poly-L-arginine hydrobromide.

6. The improvement of claim 1, wherein the method is: (a) selected from the group consisting of an immunoassay, a clinical chemistry assay, a point-of-care assay, and a lateral flow assay; (b) performed using single molecule detection; and/or (c) is adapted for use in an automated or semi-automated system.

7. (canceled)

8. (canceled)

9. A method of detecting a presence or determining an amount of a SARS-CoV-2 nucleocapsid protein in a biological sample in a subject, the method comprising:
a) contacting at least one biological sample from the subject, either simultaneously or sequentially, in any order, with:
at least one first specific binding partner comprising at least one anti-SARS-CoV antibody, anti-SARS-CoV-2 antibody, or fragment thereof thereof that specifically binds to at least one SARS-CoV-2 nucleocapsid protein in the sample,
at least one second specific binding partner comprising at least one detectable label, thereby producing one or more complexes comprising the first binding member-SARS-CoV-2 nucleocapsid protein-second specific binding partner, and
at least one polycation having a molecular weight of at least about 500 daltons or greater; and
b) assessing a signal from the one or more complexes, wherein the amount of detectable signal from the detectable label indicates the presence or amount of SARS-CoV-2 nucleocapsid protein in the sample.

10. The method of claim 9, wherein the biological sample is whole blood, serum, plasma, saliva, an oropharyngeal specimen, or a nasopharyngeal specimen.

11. The method of claim 9, wherein the first specific binding partner comprises at least one anti-SARS-CoV antibody, anti-SARS-CoV-2 antibody, or fragment thereof, or the first specific binding partner and the second specific binding partner each comprise at least one anti-SARS-CoV antibody, anti-SARS-CoV-2 antibody, or fragment thereof.

12. The method of claim 9, wherein the polycation is at least one polylysine, at least one polyornithine, at least one poly-L-histidine, at least one poly-L-arginine, at least one polyethylenimines, at least one DEAE-Dextran, or combinations thereof.

13. The method of claim 12, wherein the (i) the polylysine is poly-L-lysine hydrobromide, poly-D-lysine hydrobromide, poly-L-lysine hydrochloride, poly-L-lysine trifluoroacetate, poly(lysine, alanine) 3:1 hydrobromide, poly(lysine, arginine) 2:1 hydrobromide, poly(lysine, alanine) 1:1 hydrobromide, or poly(lysine, tryptophan) 1:4 hydrobromide; (ii) the polyornithine is poly-L-ornithine hydrobromide or poly-DL-ornithine hydrobromide; (iii) the poly-L-histidine is poly-L-histidine hydrobromide; and (iv) the poly-L-arginine is poly-L-arginine hydrochloride or poly-L-arginine hydrobromide.

14. The method of claim 9, wherein the method is: (a) selected from the group consisting of an immunoassay, a clinical chemistry assay, a point-of-care assay, and a lateral flow assay; (b) performed using single molecule detection, and/or (c) is adapted for use in an automated or semi-automated system.

15. (canceled)

16. (canceled)

17. A kit for performing the method of claim 9, wherein the kit comprises:
a. at least one specific binding partner comprising at least one anti-SARS-CoV antibody, anti-SARS-CoV-2 antibody, or fragment thereof that specifically binds to at least one SARS-CoV-2 nucleocapsid protein;
b. at least one second specific binding partner comprising at least one detectable label; and
c. at least one polycation having a molecular weight of at least about 500 daltons or greater.

18. The kit of claim 17, wherein the kit further comprises, or is configured to be used with at least one calibrator reagent, at least one control reagent, or at least one calibrator reagent and at least one control reagent.

19. The kit of claim 17, wherein the kit: (a) further comprises at least one solid support; and/or (b) is adapted for use with an automated or semi-automated system.

20. The kit of claim 17, wherein the polycation is at least one polylysine, at least one polyornithine, at least one poly-L-histidine, at least one poly-L-arginine, at least one polyethylenimines, at least one DEAE-Dextran, or combinations thereof.

21. The kit of claim 20, wherein the (i) the polylysine is poly-L-lysine hydrobromide, poly-D-lysine hydrobromide, poly-L-lysine hydrochloride, poly-L-lysine trifluoroacetate, poly(lysine, alanine) 3:1 hydrobromide, poly(lysine, arginine) 2:1 hydrobromide, poly(lysine, alanine) 1:1 hydrobromide, or poly(lysine, tryptophan) 1:4 hydrobromide; (ii) the polyornithine is poly-L-ornithine hydrobromide or poly-DL-ornithine hydrobromide; (iii) the poly-L-histidine is poly-L-histidine hydrobromide; and (iv) the poly-L-arginine is poly-L-arginine hydrochloride or poly-L-arginine hydrobromide.

22. (canceled)

23. A system for detecting a presence or determining an amount of a SARS-CoV-2 nucleocapsid protein in a biological sample in a subject, the system comprising:
at least one first specific binding partner comprising at least anti-SARS-CoV antibody, anti-SARS-CoV-2 antibody, or fragment thereof that specifically binds to at least one SARS-CoV-2 nucleocapsid protein in the sample;
at least one second specific binding partner comprising at least one detectable label, thereby producing one or more complexes comprising the first binding member-SARS-CoV-2 nucleocapsid protein-second specific binding partner;
at least one polycation having a molecular weight of at least about 500 daltons or greater; and
at least one device for detecting the at least one label from the complex.

24. The system of claim 23, wherein the device for detecting the label from the complex is automated or semi-automated.

25. The system of claim 23, wherein the polycation is at least one polylysine, at least one polyornithine, at least one poly-L-histidine, at least one poly-L-arginine, at least one polyethylenimines, at least one DEAE-Dextran, or combinations thereof.

26. The system of claim 25, wherein the (i) the polylysine is poly-L-lysine hydrobromide, poly-D-lysine hydrobromide, poly-L-lysine hydrochloride, poly-L-lysine trifluoroacetate, poly(lysine, alanine) 3:1 hydrobromide, poly(lysine, arginine) 2:1 hydrobromide, poly(lysine, alanine) 1:1 hydrobromide, or poly(lysine, tryptophan) 1:4 hydrobromide; (ii) the polyornithine is poly-L-ornithine hydrobromide or poly-DL-ornithine hydrobromide; (iii) the poly-L-histidine is poly-L-histidine hydrobromide; and (iv) the poly-L-arginine is poly-L-arginine hydrochloride or poly-L-arginine hydrobromide.

27. The method of claim 1 wherein the improvement increases sensitivity by at least about 5%, at least about 10%, at least about 15%, at least about 20%, at least about 25%, at least about 30%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 95%, at least about 100%, at least about 110%, at least about 120%, at least about 125%, at least about 130%, at least about 140%, at least about 150%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% when compared to methods which do not use or employ at least one polycation.

28. The method of claim 9 wherein the improvement increases sensitivity by at least about 5%, at least about 10%, at least about 15%, at least about 20%, at least about 25%, at least about 30%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 95%, at least about 100%, at least about 110%, at least about 120%, at least about 125%, at least about 130%, at least about 140%, at least about 150%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% when compared to methods which do not use or employ at least one polycation.
